(19) United States
(12) Patent Application Publication
(10) Pub. No.: US 2012/0291148 A1
(43) Pub. Date: Nov. 15, 2012

(54) TRANSGENIC PLANTS WITH ENHANCED CHARACTERISTICS

(75) Inventors: Christopher A. Makaroff, Oxford, OH (US); Meghan M. Holdorf, Oxford, OH (US)

(73) Assignee: Miami University, Oxford, OH (US)

(21) Appl. No.: 13/454,322

(22) Filed: Apr. 24, 2012

Related U.S. Application Data
(63) Continuation of application No. 12/061,630, filed on Apr. 2, 2008, now abandoned.
(60) Provisional application No. 60/909,649, filed on Apr. 2, 2007.

(51) Int. Cl. A01H 5/00; C12N 15/82; A01H 1/02

(52) U.S. Cl. 800/260; 800/278; 800/298; 800/301

(57) ABSTRACT

This invention relates to transgenic plants with improved phenotypic characteristics, and methods of making, identifying and selecting such plants. The transgenic plants include (a) a polynucleotide sequence encoding ETHE1 polypeptide or a functional fragment thereof, or (b) a polynucleotide that is complementary to, or hybridizes under stringent conditions to, the polynucleotide sequence of (a).

### RELATED APPLICATIONS

This application is a continuation of and claims priority to U.S. patent application Ser. No. 12/061,630, filed on Apr. 2, 2008, which claims priority to U.S. Provisional Application Ser. No. 60/909,649; filed on Apr. 2, 2007, both of which are incorporated herein by reference in their entirety.

### GOVERNMENT SUPPORT

The invention was made, at least in part, with government support from the National Science Foundation research grant MCB-9817083. The government may have certain rights in the invention.

### SEQUENCE LISTING

The instant application contains a Sequence Listing which has been submitted in ASCII format via EFS-Web and is hereby incorporated by reference in its entirety. Said ASCII copy, created on Sep. 23, 2010, is named 27876407.txt and is 108,603 bytes in size.

### BACKGROUND OF THE INVENTION

Genetic engineering has become an important and effective tool for enhancing agricultural traits in plants, including disease resistance, drought tolerance, and the protection of plants against abiotic and biotic stresses. Genetic engineering allows for the manipulation of traits by moving genes that code for advantageous traits from one species to another. Commercially successful examples of genetically engineered crops include the biofortification of vitamin A in golden rice, herbicide resistance crops for weed control, and pest control seen in bt corn. One area of economic interest is seed yield enhancement, which can be defined in terms of quantity and/or quality and is dependent on several factors including the number and size of the organs, plant architecture, seed production, root development, nutrient uptake, stress tolerance, and plant population. Optimizing one or more of the above mentioned factors can contribute to an overall increase in crop yield.

It is generally desirable to identify plant genes and polynucleotides that are involved in conferring a selective advantage on a plant, such as improving various phenotypic characteristics, including, but not limited to, growth, response to environmental challenges, yield, etc.

### SUMMARY OF THE INVENTION

The present invention relates to transgenic plants with improved phenotypic characteristics, and methods of making, identifying and selecting such plants.

Provided herein is a transgenic plant that includes a polynucleotide which contains a sequence selected from the group consisting of: (i) a polynucleotide sequence encoding ETHE1 polypeptide or a functional fragment thereof; (ii) a polynucleotide sequence that is fully complementary to the polynucleotide sequence of (i); and (iii) a polynucleotide sequence that hybridizes under stringent conditions to the polynucleotide sequence of (i) or (ii); wherein the transgenic plant exhibits at least one improved phenotypic characteristic as compared to a control plant not transformed with said polynucleotide.

In some embodiments, the transgenic plant is a dicot. In other embodiments, the transgenic plant is a monocot.

In one aspect, the improved phenotypic characteristic includes improvement in bolting rate; speed of growth, including flowering time; longevity; onset of senescence; plant size; plant biomass; vigor; thickness and uprightness of the stem; yield; stress tolerance; pathogen tolerance or a combination thereof. In some embodiments, the ETHE1 polypeptide is obtained from a plant, an animal, or a bacterium.

In one embodiment, the ETHE1 polypeptide includes a sequence having at least 50%, 60%, 70%, 80%, 90%, 95%, or 100% identity with the sequence of AtETHE1, SEQ ID NO: 46, shown in FIG. 1B.

In other embodiments, the functional fragment of the ETHE1 polypeptide is at least 200 amino acids in length and comprises one or more of the following features: (a) a deletion and/or substitution of 1 to 16 amino acids corresponding to those located at the N terminus of the AtETHE1 polypeptide sequence, SEQ ID NO: 46; (b) a deletion and/or substitution of 1 to 9 amino acids corresponding to those located at the C terminus of the AtETHE1 polypeptide sequence, SEQ ID NO: 46; (c) a deletion and/or substitution of amino acids corresponding to those at positions 36, 37, 139, 140, 141, 144, 145, and/or 146 of the AtETHE1 polypeptide sequence, SEQ ID NO: 46; and/or (d) addition of one or more amino acids between amino acid residues corresponding to positions 102 and 103, and/or between amino acid residues corresponding to positions 217 and 218 of the AtETHE1 polypeptide sequence, SEQ ID NO: 46.

Also provided herein is a method for producing a transgenic plant. The method includes: (a) providing an expression vector or cassette that includes a sequence selected from: (i) a polynucleotide sequence encoding ETHE1 polypeptide or a functional fragment thereof; (ii) a polynucleotide sequence that is fully complementary to the polynucleotide sequence of (i); or (iii) a polynucleotide sequence that hybridizes under stringent conditions to the polynucleotide sequence of (i) or (ii); and (b) transforming a plant with the expression vector or cassette, thereby producing a transgenic plant that expresses the polynucleotide sequence of (i), (ii) or (iii). A transgenic plant produced by this method exhibits improved phenotypic characteristics as compared to a control plant not transformed with the expression vector or cassette.

In one embodiment, the method further comprises selfing the transgenic plant or crossing the transgenic plant with a second plant, thereby producing progeny with improved phenotypic characteristics.

Also provided herein is a plant cell transformed with a DNA molecule that encodes an ETHE1 polypeptide or a functional fragment thereof; wherein the presence of the DNA molecule leads to overexpression of the ETHE1 polypeptide or increased ETHE1 polypeptide activity in cytoplasm of the plant cell.

Also provided herein are transgenic plants that include a plant cell transformed with a DNA molecule that encodes an ETHE1 polypeptide or a functional fragment thereof; wherein the presence of the DNA molecule leads to overexpression of the ETHE1 polypeptide or increased ETHE1 polypeptide activity in the plant cell.

In another aspect, the invention relates to a transgenic plant transformed with a gene encoding a polypeptide that regulates expression of ETHE1 polynucleotide, wherein said transgenic plant exhibits improved phenotypic characteristics as compared to a control plant not transformed with said gene.

In yet another aspect, the invention relates to a method of selecting or identifying a plant having an improved phenotypic characteristic. Such a method includes detecting the level of expression or activity of ETHE1 polynucleotide or polypeptide, wherein detecting an increase in the expression level of ETHE1 polynucleotide or polypeptide, or an increase in the activity level of ETHE1 polypeptide, is indicative of the plant having improved phenotypic characteristics as compared to a control plant where the expression or activity level of ETHE1 polynucleotide or polypeptide is not increased.

### BRIEF DESCRIPTION OF FIGURES

FIG. 1. Gene maps of AtETHE1 genomic (1A; SEQ ID NO: 44) and cDNA (1B; SEQ ID NOS 45 & 46) sequences. Exons are displayed as bolded letters. The position and direction of the primers using in these studies are shown as horizontal arrows. A T-DNA insert is shown as inverted triangles. In FIG. 1A the sequence used in the complementation clone has been underlined.

FIG. 2A. Sequence alignment of Human ETHE1 and AtGLX2-3. Protein sequences were aligned using CLUSTAL W (1.83). All sequences were compared to AtGLX2-3 labeling identical residues with black highlight and similar residues with gray highlight. The arrow shows the predicted position of the N-terminus for the AtGLX2-3 protein. Residues important for SLG catalysis are shown as asterisks. AtGLX2-3 shows 58% amino acid identity with human ETHE1. Because of the high sequence identity, AtGLX2-3 has been renamed to AtETHE1 FIG. 2A (SEQ ID NOS 47-51 respectively in order of appearance).

FIG. 2B. Sequence alignment of AtETHE1, human ETHE1, GLX2-5, and human GLX2. (ETHE1 refers to human ETHE1). The alignment was created by running a structure-based alignment with VAST between AtETHE1 and GLX2-5, and then performing a pairwise alignment between AtETHE1 and human ETHE1, and between GLX2-5 and human glyoxalase II. Residues involved in dimer formation in AtETHE1 are indicated by Δ; residues involved in substrate binding in human glyoxalase II are labelled φ; residues which belong to metal site 1 are highlighted black; residues which belong to metal site 2 are highlighted gray; and residues that have been implicated in ethylmalonic encephalopathy are marked with an asterix (SEQ ID NOS 52-55 respectively in order of appearance).

FIG. 2C. Pairwise alignment of ETHE1 Homologs. ETHE1 proteins from Arabidopsis (A. thaliana), Human, (H. sapiens), mouse (M. musculus), frog, (Xenopus laevs, Xenopus tropicalis, rice (O. sativa), zebrafish (D. rerio), and bacteria (B. phytofirmans, M. xanthus). Identical residues are highlighted in black. Positions with conserved substitutions are highlighted in gray, and residues associated with mutations observed with EE are labeled with an *. Residues are numbered from the beginning of the AtETHE1₂₅₆ protein (SEQ ID NOS 56-64 respectively in order of appearance).

FIG. 3. (a) The At1g53580 monomer. Helices are labeled 1-8 and β-strands are labeled A-L. Residues known to be involved in human ethylmalonic encephalopathy in human ETHE1 are colored pink. (b) Overlay of the AtETHE1 (cyan) and the GLX2-5 (magenta) monomers. The metal ions from the GLX2-5 structure are colored orange and the iron ion from the AtETHE1 structure is colored gray. Arrows point to changes between the folds of the two enzymes.

FIG. 4. The AtETHE1dimer. The two subunits are colored cyan and magenta.

FIG. 5. Overlay of metal-binding residues in the AtETHE1 enzyme (magenta) and the GLX2-5 enzyme (cyan). The metal ion from the AtETHE1 structure is colored purple. The metal ions from the GLX2-5 structure are colored orange. The backbone of both enzymes is also depicted in order to illustrate the unwinding of the helix near the metal-binding site. Labels correspond to AtETHE1.

FIG. 6. Overlay of the substrate-binding residues from the human glyoxalase II (cyan) with the equivalent residues in the AtETHE1 enzyme (magenta). S-Hydroxy-bromophenylcarbamoyl glutathione bound to glyoxalase II is colored yellow. Labels correspond to AtETHE1.

FIG. 7. Sequence Alignment of AtETHE1 and select metallo-β-lactamase fold proteins. Protein sequences were aligned using CLUSTAL W (1.83). Identical residues are highlighted with black and similar residues with gray highlight. Conserved metal binding ligands are indicated by closed triangles. Residues labeled with an asterisk are mutated in patients with EE. N-terminal processing site is indicated by an arrow (SEQ ID NOS 65-69 respectively in order of appearance).

FIG. 8. Gel filtration elution profile of internal protein standards and AtETHE1. Approximately 1 mg of each of the following internal protein standards was separated on a Sephacryl 5200 column: blue dextran, bovine serum albumin, ovalbumin, aldolase, and ribonuclease A. Protein containing fractions were monitored by A₂₈₀ as well as by SDS-PAGE.

FIG. 9. ¹H NMR spectrum of 1.4 mM iron-bound ETHE1 at pH 7.2. The spectra were obtained on a Bruker Avarice 500 spectrometer operating at 500.13 MHz, 298 K, and a magnetic field of 11.7 tesla, recycle delay (AQ), 41 ms; sweep width, 400 ppm. The solvent-exchangeable peak is labeled with an asterisk.

FIG. 10. EPR spectrum of 1.6 mM ETHE1 under different conditions. A, AtETHE1 as-isolated containing 0.33 equivalents of Fe; B, iron-enriched AtETHE1 containing 1.2 equivalents of Fe; C, iron-enriched AtETHE1 after 2 cycles of freeze/thaw; D, iron-enriched AtETHE1 after 4 days at 4° C. Spectra were collected on a Bruker ESP-300E spectrometer containing an ESR-900 helium flow cryostat operating at 4.7 K with 2 milliwatts of microwave power at 9.48 GHz and 10 G-field modulation at 100 kHz.

FIG. 11. Metal binding sites of (A) human glyoxalase 2 and (B) A. thaliana ETHE1.

FIG. 12. Increased Expression of AtETHE1 under various environmental stresses. RT-PCR analysis of mRNA levels of AtETHE1 under various stress conditions. Increased expression of AtETHE1 is observed under NaCl, Mannitol, and ABA stresses. The Actin 8 gene was used as an internal control

FIG. 13. AtETHE1 distribution over plant tissues. AtETHE1 is equally expressed throughout the plant. Levels of expression are a little decreased in the silique and bud.

FIG. 14. Molecular Characterization of AtETHE1. (A). Map of the ETHE1 locus. The positions and directions of primers used in this study are shown as horizontal arrows. A T-DNA insert is shown as an inverted triangle. (B) Alignment of AtETHE1 with select β-lactamase fold proteins. Identical residues are highlighted with black and similar residues with gray highlight. Residues labeled with an asterisk are conserved in ETHE1-like enzymes. Residues labeled with an inverted triangle are mutated in patients with EE. Lower case residues are metal binding ligands (SEQ ID NOS 70-74 respectively in order of appearance). (C) Phylogenic Analysis of ETHE1. The tree was derived from multiple β-lactamase fold proteins, which are aligned with Clustal W and followed by a neighbor joining phylogenetic analysis conducted with MEGA version 3.1. Bootstrap values are shown at branch points. See methods section for the accession numbers of sequences used for this analysis.

FIG. 15. Inactivation of AtETHE1 disrupts seed development. (A) Mature siliques of wild-type, AtETHE1/ethe1, and Atethe1 complementation plants observed by light microscopy. Aborted seeds are indicated by arrows. Size bar=1.5 mm. (B) AtETHE1/ethe1 developing siliques observed by light microscopy. Seeds homozygous for the ethe1 mutation are indicated with an asterisk. (C-E) SEM of developing seeds in AtETHE1/ethe1 siliques. (C) and (D) show a comparison between the outer integument in post globular-staged seeds of wild-type and the corresponding ethe1 seeds. Higher magnification image of the outer integument of an earlier staged wild-type seed is shown in panel (E). Size bar=10 μm.

FIG. 16. Embryo Development in AtETHE1/ethe1 siliques. LSCM images of Feulgan stained segregating AtETHE1/ethe1 seeds. Wild-type seeds are shown at globular (A), heart (B) and walking stick (C) stages. Atethe1 seeds from siliques with wild-type embryos shown in (A-C). The Atethe1 embryos developed to early globular (D), globular (E), and early heart (F) stages. High magnification images Atethe1 embryos at early globular (G), globular (H), and early heart (I) stages. Arrow denotes elongated cells. Size bar=20 μm.

FIG. 17. Endosperm development is abnormal in Atethe1(−/−) seeds. LSCM images of Feulgan stained segregating AtETHE1/ethe1 seeds. Endosperm development in globular-staged wild-type seed (A) showing the PEN (B) and CHZ (C) regions. Similar regions of a globular-staged ethe1 seed (D) are shown in (E-F). Endosperm development in a wild-type heart-staged seed (G) showing the PEN(H) and CHZ (I) regions. The same regions are shown in a heart-staged ethe1 seed in panels (J-L). Size bar=20 μm. e=embryo; chz=chalazal region.

FIG. 18. AtETHE1 Expression Patterns. (A) RT-PCR analysis of AtETHE1 transcript patterns in different tissues. (B) RT-PCR analysis of ETHE1 transcript levels of hydroponically grown Wt/Ws plants after exposure to environmental stresses NaCl, Mannitol, and ABA. ACT 8 was used as an internal control.

FIG. 19. Immunolocalization of AtETHE1 in Wild-type Buds. Sections of wild-type buds (A,B), anthers (C, D) or seeds (E-O) were prepared and treated with pre-immune serum (A, C, E, H, J-L) or AtETHE1 antibody (B, D, F-G, I, M-O). AtETHE1 cross reactive material is purple, non specific signals from the detection system are brown. AtETHE1 is present in the tapetal cells of the anther (B, D), zygote (F) and PEN nuclei (G). AtETHE1 is also present in globular-staged seeds (I), in particular, in the globular embryo (M), the PEN nuclei (N) and the chalazal cyst (O). The embryo and chalazal region are labeled with e and chz respectively. Size bar=50 μm.

FIG. 20. Molecular analysis of Arabidopsis ETHE1. (A) Gene map of AtETHE1. Arrows show the direction and location of primers used in this study. Bent arrows show the start and Genebank accession numbers of ESTs used for cloning. (B) Sequence alignment of AtETHE1 (SEQ ID NO: 75) with GLX2-2 (SEQ ID NO: 76) using CLUSTAL W. Amino acids in gray lettering correspond to the extended protein sequence in AtETHE1₂₉₄. which is not present in AtETHE1₂₅₆. Identical residues are highlighted in black.

FIG. 21. Localization and purification of AtETHE1₂₅₆ and AtETHE1₂₉₄ from transgenic Arabidopsis cell cultures. (A) Subcellular localization of AtETHE1₂₅₆ or AtETHE1₂₉₄ FLAG proteins in Arabidopsis cell cultures was determined through western blot analysis using cytochrome c oxidase (COX) as a mitochondrial fraction control (B) AtETHE1₂₅₆ FLAG and AtETHE1₂₉₄ FLAG proteins purified from transgenic Arabidopsis cell cultures was detected by anti-FLAG western blot analysis

FIG. 22. Peptide mapping of AtETHE1₂₉₄ using MALDI-TOF spectroscopy. MALDI-TOF spectra collected in positive ion mode of (A) trypsin digest and (B) Glu C digests of affinity purified AtETHE1₂₉₄ FLAG protein. (C) AtETHE1 protein map showing peptide coverage obtained from the MALDI-TOF spectra. Amino acids in italics correspond to the C-terminal tag attached to the protein for purification. The solid and dash underlines represent peptide matches from trypsin and Glu C digests of AtETHE1₂₉₄, respectively (SEQ ID NO: 77).

FIG. 23. Expression analysis of transgenic plants over-expressing either AtETHE1₂₅₆ or AtETHE1₂₉₄. RT-PCR analysis of AtETHE1 transcript levels in Arabidopsis plants over-expressing either (A) AtETHE1₂₅₆ or (B) AtETHE1₂₉₄. ACT 8 was used as an internal control.

FIG. 24. Potential Metabolic Routes of EMA Production. EMA levels can be elevated by the accumulation of butyryl-CoA, which can be carboxylated through propionyl-CoA carboxylase to ethylmalonyl-CoA. This is known to occur in disorders of short-chain fatty acid (3-oxidation pathways as well as through alterations in R-isoleucine catabolism. Metabolites labeled with asterisks were used in the metabolic stress studies.

FIG. 25. Effect of exogenous valine on seedling growth. (A) Light microscopy images of 7 day old AtETHE1₂₅₆-OE and wild-type seedlings grown on MS plates containing exogenous valine in concentrations from 0 mM to 1.5 mM. (B) Measurement of root lengths of plants obtained from the valine study in (A). These studies were repeated in triplicate and represent the average+/−SD of 50 plants per concentration and plant line.

FIG. 26. The effect of over-expressing either AtETHE1₂₅₆ or AtETHE1₂₉₄ on plant growth in Arabidopsis. Plants on day 19 of growth are shown for AtETHE1_{256-OE} (A) and AtETHE1₂₉₄-OE (B) respectively. The average number of days to bolting for wild-type AtETHE1₂₅₆-OE (C), and AtETHE1₂₉₄-OE plants (D). The average number of days to flowering are shown for wild-type, AtETHE1₂₅₆-OE (E) and AtETHE1₂₉₄-OE (F). The values are represented as the mean+/−SD.

FIG. 27: The effect of AtETHE1₂₅₆-OE in Arabidopsis on senescence, seed yield, dry mass, and inflorescence stem thickness. The effect of over-expression of AtETHE1₂₅₆ on time to senescence (A), Number of seeds per silique (B), the mean number of siliques per plant (C), and the mean dry mass of the full grown plant (D) was determined. Cross-sectional analysis of the primary inflorescence of AtETHE1₂₅₆-OE (E) shows an increased area compared with wild-type plants (F). Measurements represent the mean area+/−SD.

FIG. 28. Chemical profile of AtETHE1 Stems. Transmission spectra of 5 day old seedling hypocotyls of Wt and AtETHE1. Spectra are similar with the exception of two peaks (1349 cm-1 and 827 cm-1). Infrared library suggests that the peaks may be attributable to a nitrate.

FIG. 29. Effects of over-expressing Arabidopsis ETHE1₂₅₆ in Nicotiana tabacum. (A) RT-PCR of AtETHE1 in AtETHE1₂₅₆ tobacco plants transcript levels. (B) Comparison of tobacco AtETHE1₂₅₆-OE and control plants at day 17 of growth. The times to bolting, flowering, and senescence measurements are given in graphs (C), (D), and (E) respectively of AtETHE1₂₅₆-OE tobacco and the empty vector control plants. (F) Comparison of tobacco AtETHE1₂₅₆ and control plants at day 90 of growth. Mean (+/−SD) measurement of seed yield is given in (G). Statistically significant differences (p<0.05) are indicated by an asterisk.

### DETAILED DESCRIPTION

The present invention relates to transgenic plants with improved phenotypic characteristics and methods of producing, identifying and selecting such transgenic plants.

The invention will now be described with occasional reference to the specific embodiments of the invention. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular foams “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

### DEFINITIONS

As used herein, “polynucleotide” can be single stranded or double stranded DNA or RNA. The polynucleotide can be, e.g., genomic DNA or RNA, a transcript (such as an mRNA), a cDNA, a PCR product, a cloned DNA, a synthetic DNA or RNA, or the like. The polynucleotide can comprise a sequence in either sense or antisense orientations.

A “recombinant polynucleotide” is a polynucleotide that is not in its native state, e.g., the polynucleotide comprises a nucleotide sequence not found in nature, or is in a context other than that in which it is naturally found, e.g., separated from nucleotide sequences with which it typically is in proximity in nature, or adjacent (or contiguous with) nucleotide sequences with which it typically is not in proximity. For example, the sequence at issue can be cloned into a vector, or otherwise recombined with one or more additional nucleic acids.

An “isolated polynucleotide” is a polynucleotide whether naturally occurring or recombinant, that is present outside the cell in which it is typically found in nature, whether purified or not. Optionally, an isolated polynucleotide is subject to one or more enrichment or purification procedures, e.g., cell lysis, extraction, centrifugation, precipitation, or the like.

A “recombinant polypeptide” is a polypeptide produced by translation of a recombinant polynucleotide.

An “isolated polypeptide,” whether a naturally occurring or a recombinant polypeptide, is more enriched in (or out of) a cell than the polypeptide in its natural state in a wild type cell. Alternatively, or additionally, the isolated polypeptide is separated from other cellular components with which it is typically associated, e.g., by any of the various protein purification methods herein.

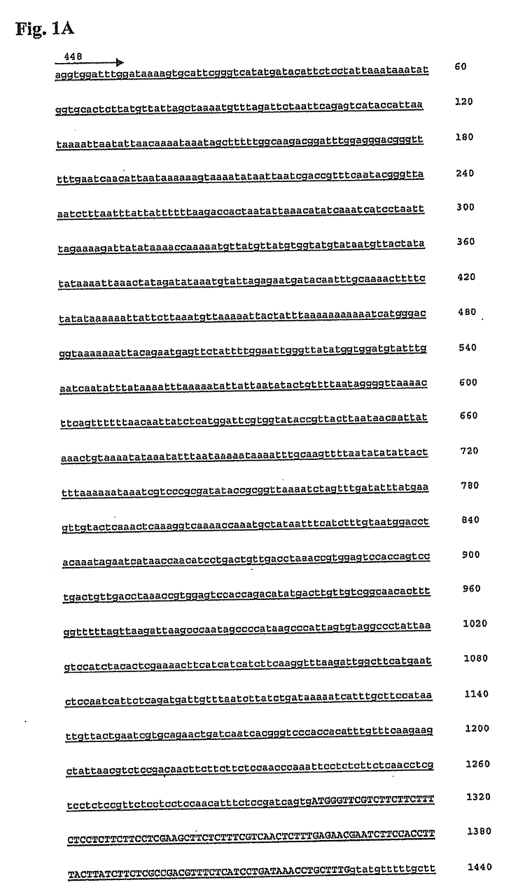

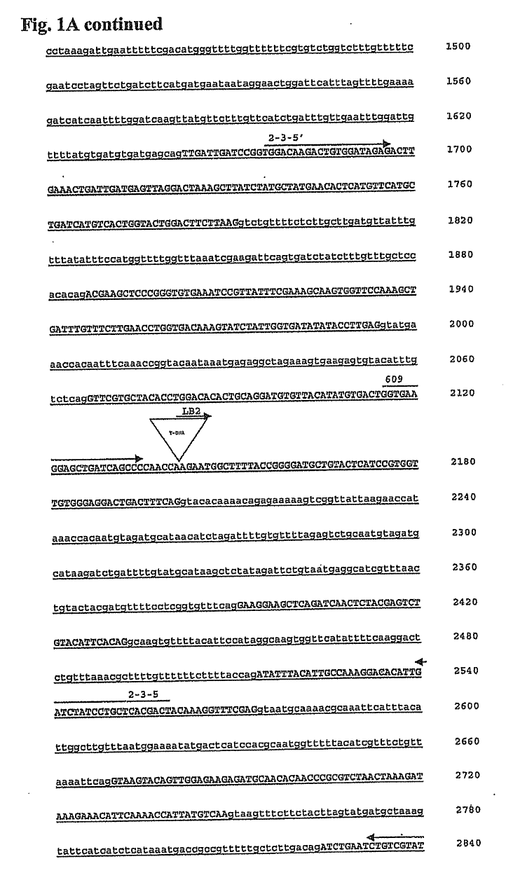

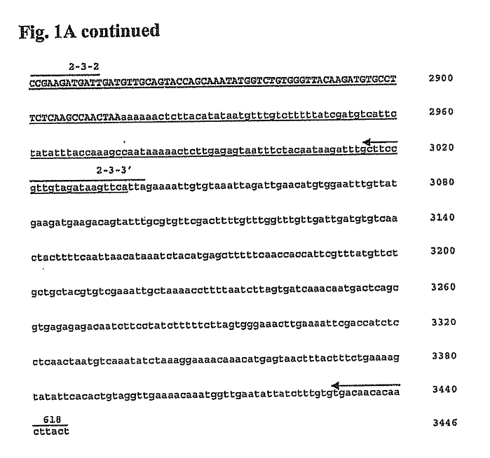

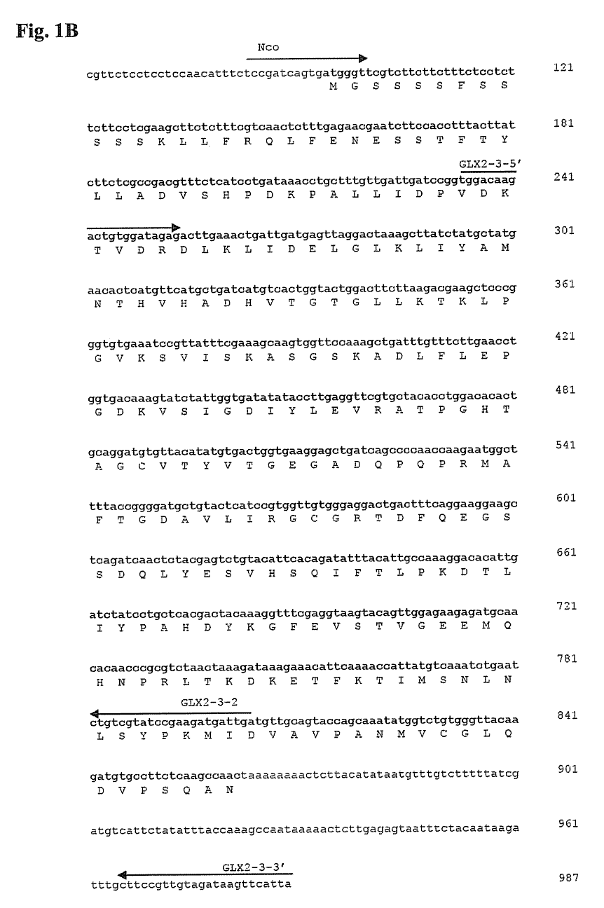

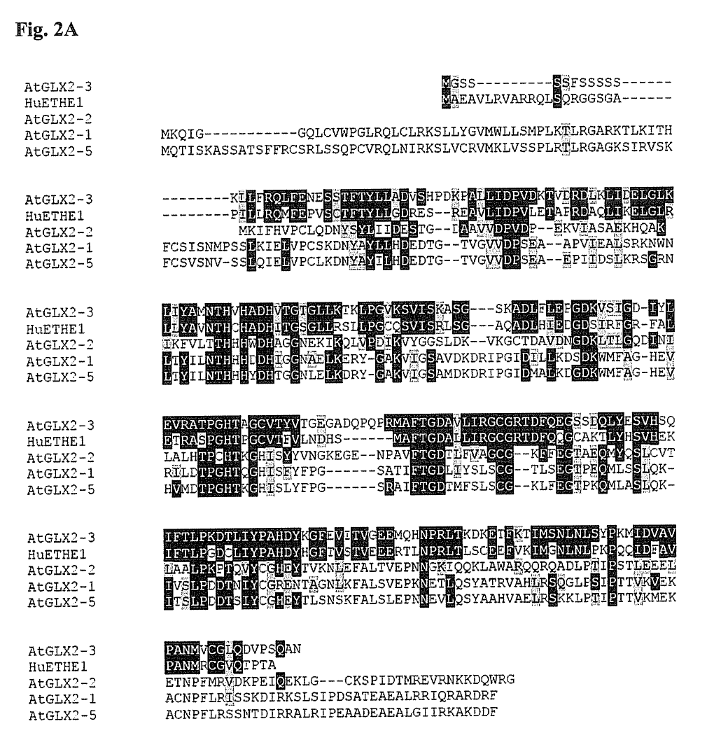

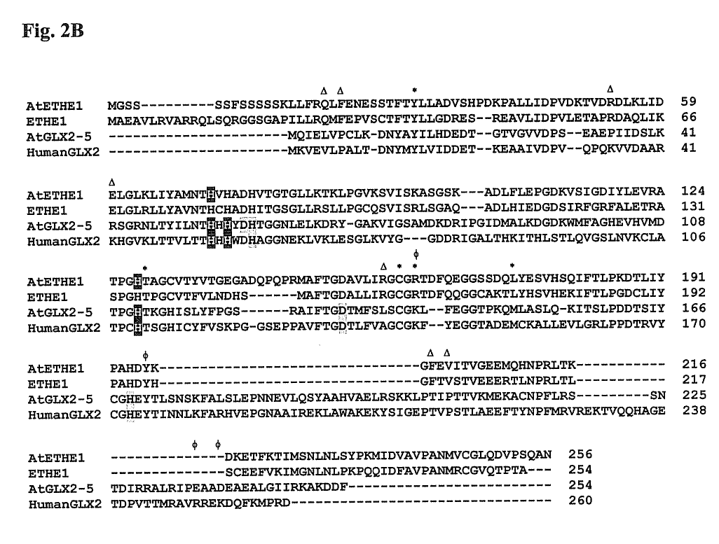

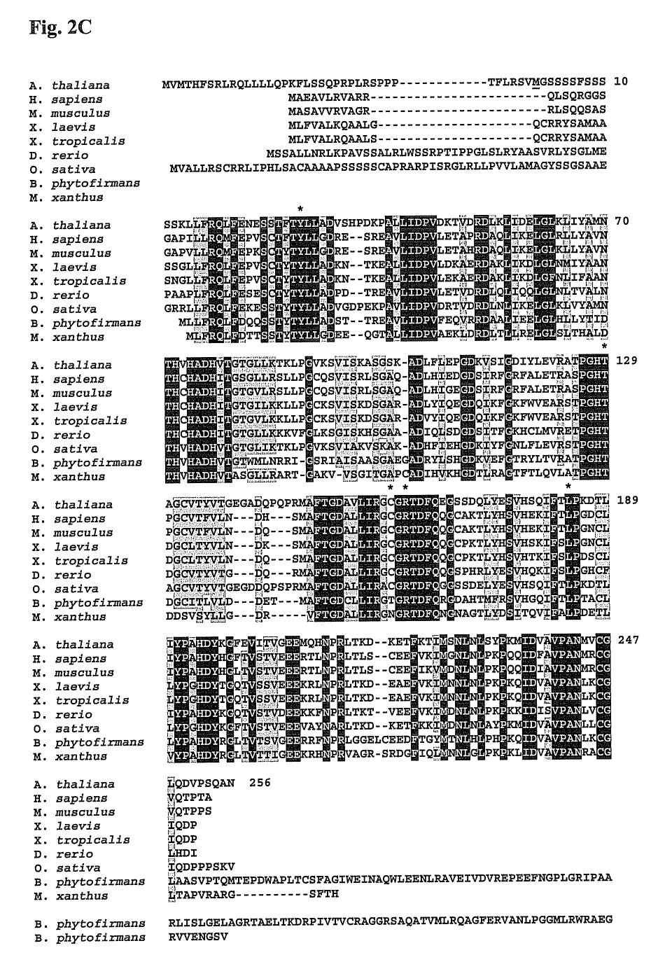

The “ETHE1 polypeptide” or “ETHE1 protein,” as used herein, comprise a polypeptide sequence that is at least 50%, 51%, 52%, 53%, 54%, 55%, 56%, 57%, 58%, 59%, 60%, 65%, 70%, 75%, 80%, 85%, 90%, 95%, 97%, 98%, 99% or 100% identical with the 256 amino acid sequence of AtETHE1, SEQ ID NO: 46, shown in FIG. 1B. An ETHE1 polypeptide can have one or more (including all) of the following conserved features: (a) contain approximately 82 conserved residues that are present in the ETHE1 proteins of organisms ranging from bacteria to humans, as shown in FIG. 2C; (b) include the metal binding amino acid residues corresponding to: H72, H74, D76, H77, H128, D153 and H194 in the AtETHE1 sequence, SEQ ID NO: 46; (c) contain those residues that comprise the ETHE1 β-lactamase fold, motif HxHxDH x₍₄₉₋₅₀₎ GHT x₍₁₄₋₂₀₎ FTGDx₍₄₀₎A/GHDY (SEQ ID NO: 1) (relative positions of the conserved amino acids are shown, metal-binding ligands are shown in bold, x denotes less highly conserved residues), (d) contain the metal binding motif H-x-[EH]-x-D-[CRSH]-X50-70-[CSD]X (SEQ ID NO: 78); (e) contain the following highly conserved residues: a tyrosine at the position equivalent to 29 in AtETHE1 (Y29), a threonine at the position equivalent to 129 in AtETHE1 (T129), a cysteine at the position equivalent to 160 in AtETHE1 (C160), an arginine at the position equivalent to 162 in AtETHE1 (R162) and a leucine at the position equivalent to 184 in AtETHE1 (L184) (these residues, when mutated, give rise to EE).

In one embodiment, the ETHE1 polypeptide has the following sequence: RQ-X-F-XXX-S-X-T-X-TYLL-X-D-X₍₅₋₇₎-A-X-LIDPV-X₍₅₎-RD-XX-L-XXX-LGL-X₍₇₎-TH-X-HADH-X-T-X₍₉₋₁₀₎-G-X₍₁₂₋₁₃₎-AD-X₍₅₎-GD-X₍₄₎-G-X₍₉₎-PGHT-X₍₁₄₋₂₀₎-FTGD-X₍₂₎-LIR-X-CGRTDFQ-X-G-X₍₇₎-S-X₍₅₎-F-X-LP-X₍₅₎-YP-X-HDY-X-G-XX-V-X₍₄₎-EE-X₍₃₎-N-X-R-X₍₇₋₉₎-F-XXX-M-X-NL-X-L-XX-P-XX-ID-XX-VPAN-XX-CG (SEQ ID NO: 2).

In other embodiments, instead of or in addition to the features above, the ETHE1 polypeptide may have one or more, including all, of the conserved and semi-conserved regions in ETHE1 homologues, as shown Table 1. In some embodiments, the ETHE1 polypeptides also contain the similar residues as outlined in FIG. 2C.

TABLE 1
Table of conserved and semi-conserved areas in
ETHE1 polypeptides

| | Relative | |
|---|---|---|
| | position | |
| | corresponding | |
| Conserved/ | to positions | |
| semi- | in AtETHE1, | |
| conserved | SEQ ID | Amino acid residues (X |
| region # | NO: 46: | denotes any amino acid) |
| 1 | 15-21 | L/F-L/F-RQ-L/M-F-E/D |
| | | (SEQ ID NO: 79) |
| 2 | 24-33 | S-S/C-T-F/Y-TYLL-A/G-D |
| | | (SEQ ID NO: 3) |
| 3 | 41-47 | A-L/V-LIDPV |
| | | (SEQ ID NO: 4) |
| 4 | 53-63 | RD-L/A-X-L-I/L-X-LGL |
| | | (SEQ ID NO: 5) |
| 5 | 70-85 | N/D-TH-V/C-HADH-V/I-T-G/ |
| | | A-T/S-G/W-L/V/I/M-L/I-K/ |
| | | R/N |
| | | (SEQ ID NO: 6) |
| 6 | 125-137 | T/S-PGHT-X-G/D-C/S-V/L/ |
| | | I-T/S-Y/F/L-V/L-T/L |
| | | (SEQ ID NO: 7) |
| 7 | 125-129 | T/S-PGHT |
| | | (SEQ ID NO: 8) |
| 8 | 149-168 | A/V-FTGD-A/C-V/L-LIR-G/ |
| | | A-C-GRTDFQ-X-G |
| | | (SEQ ID NO: 9) |
| 9 | 156-166 | A/C-V/L-LIR-G/A-C- |
| | | GRTDFQ |
| | | (SEQ ID NO: 10) |
| 10 | 173-177 | L/M-Y/F-X-S-V/I |
| | | (SEQ ID NO: 80) |
| 11 | 198-214 | G-X-E/T-V-I/S/T-T/S-V/ |
| | | I-X-EE-X-X-X-NPR-L/V-T |
| | | (SEQ ID NO: 11) |
| 12 | 188-207 | T/C-L/F-I/L/V-YP-A/G- |
| | | HDY-X-G-X-E/T-V-I/S/ |
| | | T-T/S-V/I-X-EE |
| | | (SEQ ID NO: 12) |
| 13 | 224-248 | I/V/Y/L-M-X-NL-X-L-XX- |
| | | P-XX-ID-X-A/S-VPAN-XX- |
| | | CG-L/V/I |
| | | (SEQ ID NO: 13) |

Furthermore, one of skill will recognize that individual substitutions, deletions or additions which alter, add or delete a single amino acid or a small percentage of amino acids (typically less than 5%, more typically less than 1%) in an encoded sequence are “conservative modifications” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I), Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). See also, Creighton (1984) Proteins, W. H. Freeman and Company. In addition, individual substitutions, deletions or additions which alter, add or delete a single amino acid or a small percentage of amino acids in an encoded sequence are also “conservative modifications.”

The ETHE1 polypeptide can be obtained from a number of organisms, including plants, animals, insects, eubacteria and archaebacteria. The Arabidopsis ETHE1 (AtETHE1) polypeptide has the sequence shown in FIG. 1B, SEQ ID NO: 46. The sequence has 256 amino acids starting with “MGSSS” (SEQ ID NO: 14) at the N terminus, and ending with “PSQAN” (SEQ ID NO: 15) at the C terminus. An alignment between Arabidopsis ETHE1 and the human ETHE1 (HuETHE1) polypeptide sequence is shown in FIGS. 2A & 2B. An alignment between ETHE-1 homologues from various species is shown in FIG. 2C.

As used in this application, the terms AtETHE1, At1g53580 and AtGLX2-3 are used interchangeably and denote the same molecule.

The “ETHE1 polynucleotide” is a sequence that encodes an ETHE1 polypeptide, or a functional fragment thereof. In one embodiment, the AtETHE1 polynucleotide has a sequence, SEQ ID NO: 45, that encodes the AtETHE1 polypeptide, SEQ ID NO: 46, shown in FIG. 1.

Because of the degeneracy of the genetic code, a large number of functionally identical nucleic acids encode any given polypeptide. For instance, the codons CGU, CGC, CGA, CGG, AGA, and AGG all encode the amino acid arginine. Thus, at every position where an arginine is specified by a codon, the codon can be altered to any of the corresponding codons described without altering the encoded polypeptide. Such nucleic acid variations are “silent variations,” which are one species of “conservatively modified variations.” Every polynucleotide sequence described herein which encodes a polypeptide also describes every possible silent variation, except where otherwise noted. One of skill will recognize that each codon in a nucleic acid (except AUG, which is ordinarily the only codon for methionine) can be modified to yield a functionally identical molecule by standard techniques. Accordingly, each “silent variation” of a nucleic acid which encodes a polypeptide is implicit in each described sequence.

The term “functional fragment” with respect to a polypeptide, refers to a subsequence of the polypeptide that is at least about 200, 210, 220, 230, or 240 amino acids in length. A functional fragment of ETHE-1 can include one or more (including all) of the features stated above for an ETHE1 polypeptide. In addition, the functional fragments can have one or more of the following features: (a) a deletion and/or substitution of 1 to 16 amino acids corresponding to those located at the N terminus of the AtETHE1 polypeptide sequence, SEQ ID NO: 46; (b) a deletion and/or substitution of 1 to 9 amino acids corresponding to those located at the C terminus of the AtETHE1 polypeptide sequence, SEQ ID NO: 46; (c) a deletion and/or substitution of amino acids corresponding to those at positions 36, 37, 139, 140, 141, 144, 145, and/or 146 of the AtETHE1 polypeptide sequence, SEQ ID NO: 46; (d) addition of one or more amino acids between amino acid residues corresponding to positions 102 and 103, and/or between amino acid residues corresponding to positions 217 and 218 of the AtETHE1 polypeptide sequence, SEQ ID NO: 46.

The functional fragment is a sub-sequence of the polypeptide that performs at least one biological function of the intact polypeptide in substantially the same manner, or to a similar extent, as does the intact polypeptide. In some embodiments, the functional fragment will improve the phenotypic characteristics of a plant which has been transformed with the functional fragment. In another embodiments, the functional fragment binds one Fe ion.

In reference to a nucleotide sequence, “a fragment” refers to any subsequence of a polynucleotide that encodes a functional fragment.

As used herein, the term “percent sequence identity” refers to the degree of identity between any given query sequence and a subject sequence. As used in the context of ETHE1 polypeptides, percent identity is determined by comparing the sequence of the ETHE1 polypeptide with the reference sequence AtETHE1, SEQ ID NO: 46, shown in FIG. 1B. Methods of alignment of sequences for comparison are well known in the art. Thus, the determination of percent sequence identity between any two sequences can be accomplished using a mathematical algorithm. Preferred, non-limiting examples of such mathematical algorithms are the algorithm of Myers and Miller (1988) CABIOS 4:11 17; the local homology algorithm of Smith et al. (1981) Adv. Appl. Math. 2:482; the homology alignment algorithm of Needleman and Wunsch (1970) J. Mol. Biol. 48:443 453; the algorithm of Pearson and Lipman (1988) Proc. Natl. Acad. Sci. 85:2444 2448; the algorithm of Karlin and Altschul (1990) Proc. Natl. Acad. Sci. USA 872264, modified as in Karlin and Altschul (1993) Proc. Natl. Acad. Sci. USA 90:5873 5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0); the ALIGN PLUS program (Version 3.0, copyright 1997): and GAP, BESTFIT, BLAST, FASTA, and TFASTA in the Wisconsin Genetics Software Package of Genetics Computer Group, Version 10 (available from Accelrys, 9685 Scranton Road, San Diego, Calif., 92121, USA). The scoring matrix used in Version 10 of the Wisconsin Genetics Software Package is BLOSUM62 (see Henikoff and Henikoff (1989) Proc. Natl. Acad. Sci. USA 89:10915).

Alignments using these programs can be performed using the default parameters. The CLUSTAL program is well described by Higgins et al. (1988) Gene 73:237 244 (1988); Higgins et al. (1989) CABIOS 5:151 153; Corpet et al. (1988) Nucleic Acids Res. 16:10881 90; Huang et al. (1992) CABIOS 8:155 65; and Pearson et al. (1994) Meth. Mol. Biol. 24:307 331. The ALIGN and the ALIGN PLUS programs are based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the embodiments. BLAST protein searches can be performed with the BLASTX program, score=50, wordlength=3, to obtain amino acid sequences homologous to a protein or polypeptide of the embodiments. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See the web site for the National Center for Biotechnology Information on the world wide web. Alignment may also be performed manually by inspection.

FIG. 2A. shows sequence alignment using the ClustalW (version 1.83) program with default parameters. Structural similarity was obtained using Vector Alignment Search Tool (VAST) (NCBI) (FIG. 2B). This program compares known 3D coordinates of determined protein structure to those structures in the MMDB/PDB database. (see Gibrat J F, et al., (1996) Surprising similarities in structure comparison. Curr Opin Struct Biol 6(3):377-385; Madej T, et al., (1995) Threading a database of protein cores. Proteins 23(3):356-3690.) The term “transgenic plant” refers to a plant that contains genetic material not found in a wild type plant of the same species, variety or cultivar. Typically, the foreign genetic material has been introduced into the plant by human manipulation.

A transgenic plant may contain an expression vector or cassette. The expression cassette typically comprises a polypeptide-encoding sequence operably linked (i.e., under regulatory control of) to appropriate inducible or constitutive regulatory sequences that allow for the expression of the polypeptide. The expression cassette can be introduced into a plant by transformation or by breeding after transformation of a parent plant. A plant refers to a whole plant as well as to a plant part, such as seed, fruit, leaf, or root, plant tissue, plant cells or any other plant material, e.g., a plant explant, as well as to progeny thereof, and to in vitro systems that mimic biochemical or cellular components or processes in a cell.

The term “regulatory region” refers to nucleotide sequences that, when operably linked to a sequence, influence transcription initiation or translation initiation or transcription termination of said sequence and the rate of said processes, and/or stability and/or mobility of a transcription or translation product. As used herein, the term “operably linked” refers to positioning of a regulatory region and said sequence to enable said influence. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns. Regulatory regions can be classified in two categories, promoters and other regulatory regions.

The phrase “ectopically expression or altered expression” in reference to a polynucleotide indicates that the pattern of expression in, e.g., a transgenic plant or plant tissue, is different from the expression pattern in a wild type plant or a reference plant of the same species. For example, the polynucleotide or polypeptide is expressed in a cell or tissue type other than a cell or tissue type in which the sequence is expressed in the wild type plant, or by expression at a time other than at the time the sequence is expressed in the wild type plant, or by a response to different inducible agents, such as hormones or environmental signals, or at different expression levels (either higher or lower) compared with those found in a wild type plant. The resulting expression pattern can be transient or stable, constitutive or inducible.

In reference to the ETHE1 polypeptide, the term “ectopic expression or altered expression” may further relate to altered activity levels resulting from the interactions of the polypeptides with exogenous or endogenous modulators or from interactions with factors or as a result of the chemical modification of the polypeptides.

In some embodiments, the term “ectopic or altered expression” refers to expression of the ETHE1 polypeptide, or functional fragment thereof, in the cytoplasm.

The term “phenotypic characteristic” refers to a physiological, morphological, biochemical or physical characteristic of a plant or particular plant material or cell. These characteristics include bolting rate; speed of growth, including flowering time; longevity of the plant, including longevity of flowers; onset of senescence; plant size; plant biomass; vigor; thickness and uprightness of the stem; yield, including seed yield, seed size and number; stress tolerance; pathogen tolerance; etc.

In some instances, the characteristic is visible to the human eye, such as seed or plant size, or can be measured by available biochemical techniques, such as the protein, starch or oil content of seed or leaves or by the observation of the expression level of genes, e.g., by employing Northern analysis, RT-PCR, microarray gene expression assays or reporter gene expression systems, or by agricultural observations such as stress tolerance, yield or pathogen tolerance.

The term “altered” or “improved” phenotypic characteristic refers to a detectable improvement in a characteristic in a plant ectopically expressing a polynucleotide or polypeptide of the present invention relative to a plant not doing so, such as a wild type plant. In some cases, the trait modification can be evaluated quantitatively. For example, the improved phenotypic characteristic can entail at least about a 2% increase or decrease in an observed trait (difference), at least a 5% difference, at least about a 10% difference, at least about a 20% difference, at least about a 30%, at least about a 50%, at least about a 70%, or at least about a 100%, at least a 300% or an even greater difference. It is known that there can be a natural variation in the characteristic. Therefore, the improved characteristic observed entails a change of the normal distribution of the trait in the plants compared with the distribution observed in wild type plant.

As used herein, “biomass” refers to useful biological material including a product of interest, which material is to be collected and is intended for further processing to isolate or concentrate the product of interest. “Biomass” may comprise the fruit or parts of it or seeds, leaves, flowers, stems or roots where these are the parts of the plant that are of particular interest for the industrial purpose. “Biomass”, as it refers to plant material, includes any structure or structures of a plant that contain or represent the product of interest.

As used herein, “vigor” refers to the plant characteristic whereby the plant emerges from soil faster, has an increased germination rate (i.e., germinates faster), has faster and larger seedling growth, flowers faster and/or germinates faster under cold conditions as compared to the wild type or control under similar conditions.

A “plant” as used herein includes whole plants, shoot vegetative organs/structures (e.g., leaves, stems and tubers), roots, flowers and floral organs/structures (e.g., bracts, sepals, petals, stamens, carpels, anthers and ovules), seed (including embryo, endosperm, and seed coat) and fruit (the mature ovary), plant tissue (e.g., vascular tissue, ground tissue, and the like) and cells (e.g., guard cells, egg cells, and the like), and progeny of same. The class of plants that can be used in the invention is generally as broad as the class of higher and lower plants amenable to transformation techniques, including angiosperms (monocotyledonous and dicotyledonous plants), gymnosperms, ferns, horsetails, psilophytes, lycophytes, bryophytes, and multicellular algae.

“Stringency,” as used herein is a function of nucleic acid molecule probe length, nucleic acid molecule probe composition (G+C content), salt concentration, organic solvent concentration and temperature of hybridization and/or wash conditions. Stringency is typically measured by the parameter Tₘ, which is the temperature at which 50% of the complementary nucleic acid molecules in the hybridization assay are hybridized, in terms of a temperature differential from Tₘ. High stringency conditions are those providing a condition of Tₘ−5° C. to Tₘ−10° C. Medium or moderate stringency conditions are those providing Tₘ−20° C. to Tₘ−29° C. Low stringency conditions are those providing a condition of Tₘ−40° C. to Tₘ−48° C. The relationship between hybridization conditions and Tₘ (in ° C.) is expressed in the mathematical equation: Tₘ=81.5−16.6(log₁₀ [Na⁺])+0.41(% G+C)−(600/N) (I) where N is the number of nucleotides of the nucleic acid molecule probe. This equation works well for probes 14 to 70 nucleotides in length that are identical to the target sequence. The equation below, for Tₘ of DNA-DNA hybrids, is useful for probes having lengths in the range of 50 to greater than 500 nucleotides, and for conditions that include an organic solvent (formamide): Tₘ=81.5+16.6 log {[Na⁺]/(1+0.7[Na⁺])}+0.41(% G+C)−500/L0.63(% formamide) (II) where L represents the number of nucleotides in the probe in the hybrid (21). The Tₘ of Equation II is affected by the nature of the hybrid: for DNA-RNA hybrids, Tₘ is 10-15° C. higher than calculated; for RNA-RNA hybrids, Tₘ is 20-25° C. higher. Because the Tₘ decreases about 1° C. for each 1% decrease in homology when a long probe is used (Frischauf et al. (1983) J. Mol Biol, 170: 827-842), stringency conditions can be adjusted to favor detection of identical genes or related family members.

Equation II is derived assuming the reaction is at equilibrium. Therefore, hybridizations according to the present invention are most preferably performed under conditions of probe excess and allowing sufficient time to achieve equilibrium. The time required to reach equilibrium can be shortened by using a hybridization buffer that includes a hybridization accelerator such as dextran sulfate or another high volume polymer.

Stringency can be controlled during the hybridization reaction, or after hybridization has occurred, by altering the salt and temperature conditions of the wash solutions. The formulas shown above are equally valid when used to compute the stringency of a wash solution. Preferred wash solution stringencies lie within the ranges stated above; high stringency is 5-8° C. below Tₘ, medium or moderate stringency is 26-29° C. below Tₘ and low stringency is 45-48° C. below Tₘ.

### DESCRIPTION

The inventors have found that the ETHE1 polynucleotides or polypeptides are useful for modifying plant characteristics when the expression levels of the polynucleotide or the polypeptide are increased, or when the ETHE1 polypeptide is expressed in the cytoplasm, as compared with a wild type plant.

Accordingly, in one aspect, the invention includes a transgenic plant that has been transformed with a polynucleotide selected from the group consisting of: (i) a polynucleotide sequence encoding an ETHE1 polypeptide or a functional fragment thereof; (ii) a polynucleotide sequence that is fully complementary to the polynucleotide sequence of (i); and (iii) a polynucleotide sequence that hybridizes under stringent conditions to the polynucleotide sequence of (i) or (ii). Such a transgenic plant exhibits improved phenotypic characteristics as compared to a control plant not transformed with the polynucleotide.

Exogenous genetic material may be transferred into a plant by the use of a DNA construct designed for such a purpose by methods that utilize Agrobacterium, particle bombardment or other methods known to those skilled in the art. Design of such a DNA construct is generally within the skill of the art (Plant Molecular Biology: A Laboratory Manual, eds. Clark, Springer, New York (1997). Examples of such plants into which exogenous genetic material may be transferred, include, without limitation, alfalfa, Arabidopsis, barley, Brassica (e.g. broccoli, cabbage), citrus, cotton, garlic, oat, oilseed rape, onion, canola, flax, maize, an ornamental annual and ornamental perennial plant, pea, peanut, pepper, potato, rice, rye, sorghum, soybean, strawberry, sugarcane, sugar beet, tomato, wheat, poplar, pine, fir, eucalyptus, apple, lettuce, lentils, grape, banana, tea, turf grasses, sunflower, oil palm, Phaseolus, trees, shrubs, vines, etc. It is well known that agronomically important plants comprise genotypes, varieties and cultivars, and that the methods and compositions of the present invention can be tested in these plants by those of ordinary skill in the art of plant molecular biology and plant breeding.

Examples of means by which transformation can be accomplished are well known in the art. Some examples are described in the Examples. Other methods of transformation include, for example, Agrobacterium-mediated transformation (of dicots (Needleman and Wunsch (1970) J. Mol. Biol. 48:443; Pearson and Lipman (1988) Proc. Natl. Acad. Sci. (USA) 85: 2444), of monocots (Yamauchi et al. (1996) Plant Mol. Biol. 30:321-9; Xu et al. (1995) Plant Mol. Biol. 27:237; Yamamoto et al. (1991) Plant Cell 3:371), and biolistic methods (P. Tijessen, “Hybridization with Nucleic Acid Probes” In Laboratory Techniques in Biochemistry and Molecular Biology, P. C. vand der Vliet, ed., c. 1993 by Elsevier, Amsterdam), electroporation, in planta techniques, and the like.

Monocotyledonous plants (monocots) are transformable by a number of methods, including Agrobacterium-mediated gene transfer (Yamauchi et al. (1996) Plant Mol. Biol. 30:321-9; Xu et al. (1995) Plant Mol. Biol. 27:237; Yamamoto et al. (1991) Plant Cell 3:371; Arochiasamy and Ignacimuthu 2007 Plant Cell Rep 26:1745-1753; Wu et al. 2007 Transgenic Res, July 19; Vega et al. 2008 Plant Mol Biol (2008) 66:587-598) polyethylene glycol mediated transformation (Datta et al. 1992 Plant Mol Biol 20:619-629), particle bombardment (Christou 1997 Plant Mol Biol 35:197-203), and electroporation (Tada et al. 1991 EMBO 10:1803-1808). Agrobacterium-mediated transformation is facilitated by the natural capacity of Agrobacterium to transfer genetic material from its tumor-inducing (Ti) plasmid, referred to as T-DNA, into the genome of a host plant. Generally, in Agrobacterium-mediated transformation, the gene or construct of interest is inserted into a suitable DNA vector and transformed into Agrobacteria. The Agrobacteria containing the vector are grown in a liquid medium and, in some embodiments, centrifuged and resuspended in a preinduction medium. Plant tissues or immature plant embryos are submerged and incubated in the culture of Agrobacteria. Excess bacteria are removed and the tissues or embryos are transferred to a cocultivation medium and incubated. In some embodiments, the plant tissues or embryos are then washed. In some embodiments, the plant tissues or embryos are then transferred to a selection medium which may contain an antibiotic to kill the Agrobacteria, an herbicide to kill untransformed plant tissue, or both. The plant tissue or embryo is then cultured as appropriate to facilitate growth into a viable plant.

Suitable DNA vectors for Agrobacterium-mediated transformation have the necessary functionality of the T-DNA transfer function of the Ti plasmid and the ability to stably replicate in Agrobacterium. In some embodiments, these vectors are also stably maintained in E. coli. In some embodiments, these vectors comprise a selectable marker or reporter gene. In some embodiments, the vector is selected based on the host plant to be targeted. Many suitable vectors are know in the art, and include the pRSTI plasmid for rice (Terada et al. 2004 Plant Cell Rep 22:653-659), pSoup/pGreen plasmids for wheat (Hellens et al. 2000 Plant Mol Biol 42:819-832; Wu et al. 2007 Transgenic Res, July 19); and binary bacterial artificial chromosome vector (BIBAC) or competent artificial chromosomes (TAC) for corn (Vega et al. 2008 Plant Mol Biol (2008) 66:587-598).

In some embodiments, the Agrobacterium used for the transformation are selected from Agrobacterium tumefaciens and Agrobacterium rhizogens. Suitable media for Agrobacterium cultures include, but are not limited to, CM4C liquid medium (Cheng et al. 1997 Plant Physiol 115:971-980). Suitable preinduction media include, but are not limited to, AA medium (Toriyama and Hinata 1985 Plant Sci 41:179-183), comprising 20 g/l sucrose, 1 mg/l 2,4-D, acetosyringone 50 μM, pH 5.8

Incubation times for exposing the plant tissue to the Agrobacterium can range from 10 minutes to 100 hours, including, for example, 20 minutes, 30 minutes, 40 minutes, 50 minutes, 1 hour, 2 hours, 3 hours, 4 hours, 6 hours, 8 hours, 10 hours, 12, hours, 18 hours, 24 hours, 48 hrs, or 72 hours. In some embodiments, this incubation is performed in the dark.

Incubation in the cocultivation media can range from 5 minutes to 7 days, including, for example, 10 minutes, 15 minutes, 20 minutes, 30 minutes, 1 hour, 2 hours, 3 hours, 4 hours, 6 hours, 8 hours, 10 hours, 12, hours, 18 hours, 1 day, 2 days, 3 days, 4 days, 5 days, or 6 days. Cocultivation of Agrobacterium and monocot tissues in vitro may be facilitated with vacuum infiltration, for example by applying a vacuum of about 1 mm Hg to about 5 mm Hg for about 5 to about 20 minutes. In one embodiment, a vacuum of about 2 mm Hg is applied for about 10 to about minutes.

Media for co-cultivation with Agrobacterium are known in the art and disclosed for example by Jones et al. 2005 J Ceral Sci 41:137-147. For example, a suitable medium for cocultivation is AAM (Hiei et al. (1994) Plant J. 6:271). Examples of co-cultivation media for vacuum infiltration are described by Bechtold et al. (1993) Life Sci 316: 1194 and Bent et al. (1994) Science 265:1856, the disclosures of which are incorporated herein by reference. In another embodiment, the co-cultivation medium for vacuum infiltration additionally contains a surfactant as a wetting agent, for example a liquid silicone-polyether copolymer such as SilwetOL-77 (a mixture of polyalkyleneoxide modified heptamethyltrisiloxane (84%) and allyloxypolyethylene glycol methyl ether (16%) available from OSi Specialities Sistersville, W). The cocultivation medium also preferably contains acetosyringone, which is a known inducer of the vir region genes.

A representative semi-solid medium for co-cultivation contains half strength MS major salts, MS minor salts, MS vitamins, 300 mg/l casamino acid, 500 mg/l L-proline, 30 g/l sucrose, 2.25 g/l phytagel or 7 g/l agar, pH 5.8, and 400 μM acetosyringone (SA-AS) (Arockiasamy and Ignacimuthu 2007 Plant Cell Rep 26:1745-1753).

Suitable plant tissues for transformation include, but are not limited to, leaves, roots, hypocotyls, petioles, cotyledons, seeds, shoot apices, scutella, calli, cell suspensions induced from scutella, immature embryos, and inflorescence.

In each of the foregoing methods, successful transformation may be monitored by selection and screening. As described hereinabove, the engineered Agrobacterium strains used in the present methods generally contain a selectable marker gene that encodes a product that allows detoxification or evasion of a selective agent, such as an antibiotic or herbicide. Selection for transformants is accomplished by applying the appropriate selective agent to the culture medium, soil or plantlet in concentrations known in the art, and selecting tissues or plants that survive the selection agent. For example, putatively transformed monocots may be allowed to flower and set seeds, and seeds are germinated in selection medium to identify transformant seedlings.

Transformation may also be monitored by screening for the expression of a reporter gene or the heterologous nucleic acid. The screening method is dependent upon the product encoded by the reporter gene or heterologous gene of interest. The heterologous gene may provide the screenable marker, or a nucleic acid encoding a screenable marker may be present in addition to the desired heterologous gene. Reporter genes encode products that can be directly detected, or that catalyze reactions having detectable products. Expression of reporter genes can often be measured visually or biochemically. Suitable-reporter genes and detection methods useful in plants are well known in the art, and reviewed for example by Schrott in Gene Transfer to Plants, Potrykus et al., eds, Springer-Verlag, Berlin, 1995, p. 325.

Successful transformation of monocots by the present methods may also be confirmed by genomic analysis. For example, in Southern blot analysis, genomic DNA of putatively transformed plants is digested with restriction enzymes, fractionated on an agarose gel, blotted to a nitrocellulose membrane, and probed with a labeled DNA fragment of a plasmid in the Agrobacterium, for example a fragment from a gene encoding a selectable or screenable marker, or the heterologous nucleic acid. Additional methods of characterization of transgenic plants by molecular analysis, for example by Northern blot analysis, immunoblot analysis, and PCR amplification, are also known in the art, and described for example by Buchholz et al (1998) in Plant Virology Protocols: From Virus Isolation to Transgenic Resistance Foster et al., eds, Humana Press, Inc., Totowa, N.J., pp. 383-396.

Other transformation methods known in the art include: transformation by particle bombardment (Christou 1997 Plant Mol Biol 35:197-203); polyethylene glycol mediated transformation (Maniatis et al. 1982; Kerns et al. 1982; Peng et al. 1991 Rice Genetics II p. 563-574); electroporation (Tada et al. 1991 EMBO 10:1803-1808, EP0564595; H. Potter et al., PNAS USA, 81, 7161 (1984).).

The recombinant DNA present in the electroporation buffer may be in supercoiled, linear single- or double-stranded form. In preferred embodiments, linear double-stranded DNA, e.g., from recombinant plasmids, is employed. Although concentrations as low as about 1 μg per 1.0 ml of electroporation buffer may be used, it is preferred that DNA be at a concentration of about 100 μg/ml or greater. These amounts represent about 1-100 μg of DNA per 0.1 ml of packed cells.

DNA vectors or ballistic methods can be used to transfer the polynucleotide. This may be done by operably linking the coding sequences of the polynucleotide to appropriate plant promoters. Constitutive promoters (such as the Cauliflower mosaic virus 35S promoter), tissue specific promoters (such as the patatin promoter) or non-constitutive promoters could all be used, with particular advantages in particular cases.

For example, the transformed plants may be more resistant to certain abiotic stresses than the untransformed plants. Therefore, the plant promoter used may be one that is induced by stress. Such promoters are known in the art, e.g. LTI78 (Nordin et al. [1993] Plant Mol. Biol. 21, 641-653) and RAB18 (L.ang.ng & Palva [1992] Plant Mol. Biol. 20, 951-962).

In one embodiment, the transgenic plant is transformed with an ETHE1 polypeptide that comprises a sequence having at least 50% identity with the sequence of AtETHE1, SEQ ID NO: 46, shown in FIG. 1B.

In another embodiment, the ETHE1 polypeptide comprises a sequence having at least 60% identity with the sequence of AtETHE1, SEQ ID NO: 46, shown in FIG. 1B.

In another embodiment, the ETHE1 polypeptide comprises a sequence having at least 70% identity with the sequence of AtETHE1, SEQ ID NO: 46, shown in FIG. 1B.

In yet another embodiment, the ETHE1 polypeptide comprises a sequence having at least 80% identity with the sequence of AtETHE1, SEQ ID NO: 46, shown in FIG. 1B.

In yet another embodiment, the ETHE1 polypeptide comprises a sequence having at least 90% identity with the sequence of AtETHE1, SEQ ID NO: 46, shown in FIG. 1B.

In yet another embodiment, the ETHE1 polypeptide comprises a sequence having at least 95% identity with the sequence of AtETHE1, SEQ ID NO: 46, shown in FIG. 1B.

In yet another embodiment, the ETHE1 polypeptide comprises a sequence having 100% identity with the sequence of AtETHE1, SEQ ID NO: 46, shown in FIG. 1B. In some embodiments, the ETHE1 polypeptide has one or more of the following conserved features: (a) contain approximately 82 conserved residues that are present in the ETHE1 proteins of organisms ranging from bacteria to humans, as shown in FIG. 2C; (b) include the metal binding amino acid residues corresponding to: H72, H74, D76, H77, H128, D153 and H194 in the AtETHE1 sequence, SEQ ID NO: 46; (c) contain those residues that comprise the ETHE1 β-lactamase fold, motif HxHxDH x₍₄₉₋₅₀₎ GHT x₍₁₄₋₂₀₎ FTGDx₍₄₀₎A/GHDY (SEQ ID NO: 1) (relative positions of the conserved amino acids are shown, metal-binding ligands are shown in bold, x denotes less highly conserved residues), (d) contain the metal binding motif H-x-[EH]-x-D-[CRSH]-X50-70-[CSD]X (SEQ ID NO: 78); (e) contain the following highly conserved residues: a tyrosine at the position equivalent to 29 in AtETHE1 (Y29), a threonine at the position equivalent to 129 in AtETHE1 (T129), a cysteine at the position equivalent to 160 in AtETHE1 (C160), an arginine at the position equivalent to 162 in AtETHE1 (R162) and a leucine at the position equivalent to 184 in AtETHE1 (L184) (these residues, when mutated, give rise to EE).

In some embodiments, the ETHE1 polypeptide can also contain the similar residues as outlines in FIG. 2C, which are found between different ETHE-1 homologues from different species.

In some embodiments, the functional fragment of the ETHE1 polypeptide is at least about 200, 210, 220, 230, or 240 amino acids in length.

In some embodiments, the functional fragment of ETHE-1 can include one or more (including all) of the features stated above for an ETHE1 polypeptide.

In other embodiment, or in addition, the functional fragments can have one or more of the following features: (a) a deletion and/or substitution of 1 to 16 amino acids corresponding to those located at the N terminus of the AtETHE1 polypeptide sequence, SEQ ID NO: 46; (b) a deletion and/or substitution of 1 to 9 amino acids corresponding to those located at the C terminus of the AtETHE1 polypeptide sequence, SEQ ID NO: 46; (c) a deletion and/or substitution of amino acids corresponding to those at positions 36, 37, 139, 140, 141, 144, 145, and/or 146 of the AtETHE1 polypeptide sequence, SEQ ID NO: 46; and/or (d) addition of one or more amino acids between amino acid residues corresponding to positions 102 and 103, and/or between amino acid residues corresponding to positions 217 and 218 of the AtETHE1 polypeptide sequence, SEQ ID NO: 46.

In some embodiments, the functional fragment performs at least one biological function of the intact ETHE1 polypeptide in substantially the same manner, or to a similar extent, as does the intact ETHE1 polypeptide. In some embodiments, the functional fragment will improve the phenotypic characteristics of a plant which has been transformed with the functional fragment. In other embodiments, the functional fragment can bind one Fe ion.

The improved phenotypic characteristics of the transgenic plant can include, but are not limited to, improved: bolting rate; speed of growth, including flowering time; longevity; onset of senescence; plant size; plant biomass; vigor; thickness and uprightness of the stem; yield; stress tolerance; pathogen tolerance or a combination thereof.

Plants that can benefit from such improved phenotypic characteristics include trees, such as timber trees, including, but not limited to poplar, pine, walnut and cottonwood. For example, the increased speed of growth can reduce time to harvest.

Other plants include seed crops, including but not limited to soybean, corn, wheat rice, canola. The improved characteristics can include increased speed of growth and increased seed yield.

Another category of plants are annual or perennial ornamentals, where increased speed of growth, reduced time in green house, early flowering and longevity of flowers, and reduced time to harvest can be beneficial.

Similarly, annual vegetable plants can benefit from speedier growth, reduced time in green house and reduced time to harvest.

The invention also relates to a method for producing a transgenic plant with improved phenotypic characteristics. The method includes: providing an expression vector or cassette that comprises either: (i) a polynucleotide sequence encoding an ETHE1 polypeptide or a functional fragment thereof; or (ii) a polynucleotide sequence that is fully complementary to the polynucleotide sequence of (i); or (iii) a polynucleotide sequence that hybridizes under stringent conditions to the polynucleotide sequence of (i) or (ii). Then, the method includes transforming a plant with the expression vector or cassette, thereby producing a transgenic plant that expresses the polynucleotide sequence of (i), (ii) or (iii). A transgenic plant thus produced can exhibit the improved phenotypic characteristics as compared to a control plant not transformed with the expression vector or cassette.

The expression vector can further comprise a constitutive, inducible, or tissue-active promoter operably linked to the polynucleotide sequence. In one embodiment, the promoter is 35S promoter.

Methods of transformation of monocots and dicots are well known in the art and two embodiments are described in the Examples below.

The method for producing a plant having improved phenotypic characteristics can further include selfing the transgenic plant or crossing the transgenic plant with a second plant, to produce progeny with improved phenotypic characteristics. Progeny can include seeds or other propagation material that will produce plants with improved phenotypic characteristics.

The invention also relates to a plant cell transformed with a DNA molecule that encodes an ETHE1 polypeptide or a functional fragment thereof, wherein the presence of the DNA molecule leads to over expression of ETHE1 polypeptide, or increased polypeptide activity in the cytoplasm of the plant cell.

Transgenic plants obtained from the transformed cell described above, and propagation material and seed from such a transgenic plants are also contemplated herein.

The transgenic plant or plant cell of the present invention can be either a monocot or a dicot.

Another means of producing a transgenic plant with improved phenotypic characteristics includes transforming a plant with a polynucleotide that encodes a polypeptide that regulates expression of the ETHE1 polynucleotide. Such a polypeptide can be, for example, a recombinantly produced polypeptide comprising a zinc finger domain, which is specific for the regulatory element, and an effector domain, which can be a repressor domain or an activator domain. Thus, the invention also provides a transgenic plant produced by this method, as well as to a plant cell obtained from such transgenic plant, wherein said plant cell exhibits improved phenotypic characteristics. Other material, such as a plant cell or seed from such transgenic plant, is also contemplated.

The polypeptide that regulates expression of the ETHE1 polypeptide can increase the expression or activity of the ETHE1 polypeptide generally, or increase the expression or activity of the ETHE1 polypeptide in the cytoplasm of the transgenic plant cells.

The polynucleotide encoding the polypeptide can be operatively linked to and expressed from a constitutively active, inducible or tissue specific or phase specific regulatory element.

The invention also relates to a method of selecting or identifying a plant having an improved phenotypic characteristic. The method includes a method of detecting the level of expression of the ETHE1 polynucleotide or polypeptide; or detecting the level activity of the ETHE1 polypeptide in a plant cell. Detecting an increase in the expression level of the ETHE1 polynucleotide or polypeptide, or an increase in the activity level of ETHE1 polypeptide, is indicative of the plant having improved phenotypic characteristics as compared to a control plant where the expression or activity level of the ETHE1 polynucleotide or polypeptide is not increased.

In one embodiment, the method includes detecting the expression level of the ETHE1 polynucleotide or polypeptide or activity level of the ETHE1 polypeptide in the cytoplasm of a plant cell.

Examples of assays to detect the ETHE1 polynucleotide level include, but are not limited to, Northern Blotting, RT-PCR, microarray gene expression assays, or reporter gene expression systems. Examples of assays to detect the ETHE1 polypeptide level include, but are not limited to Western Blotting or ELISA.

Such a method is useful for marker-assisted breeding. Therefore, an additional aspect of the invention provides a method for marker-assisted breeding to select plants having an altered phenotypic characteristic comprising the method described above.

In one embodiment, this method can also include the additional steps of selfing the selected plant or crossing the selected plant with a second plant, thereby producing progeny with improved phenotypic characteristics. Progeny can include seeds or other propagation material that will produce plants with improved phenotypic characteristics.

The following examples describe the characterization of ETHE1 and show that it is an essential gene in plants. Example 1 describes sequence homology of AtETHE1 to other ETHE1 polypeptides. Examples 2 and 3 show the physical structure and chemical properties of the AtETHE1 polypeptide. Example 4 observes enhanced expression of AtETHE1 in response to abiotic stresses. Example 5 describes the essential role of AtETHE1 in embryonic plant development. Example 6 describes a method of producing AtETHE1 over-expression Arabidopsis thaliana and tobacco plants and the phenotypic characteristics of theses plants, as well as the chemical and metabolic differences between the AtETHE1 over-expression and wild-type plants.

The examples of the present disclosure presented below are provided only for illustrative purposes and not to limit the scope of the disclosure. Numerous embodiments of the disclosure within the scope of the claims that follow the examples will be apparent to those of ordinary skill in the art from reading the foregoing text and following examples.

### Example 1

### At1g53580 Homology with huETHE1

Five putative glyoxalase II isozymes have been identified in the Arabidopsis genome based on sequence homology: GLX2-1 (At2g43430), GLX2-2 (At3g10850), GLX2-3 (At1g53580), GLX2-4 (At1g06130), and GLX2-5 (At2g31350). GLX2-2 is cytosolic, while GLX2-1, 2-4, and 2-5 are mitochondrial enzymes.

A detailed sequence comparison showed that At1g53580 has greater sequence identity (54%) to the human gene ETHE1 (huETHE1), a gene responsible for the recessive autosomal disorder, Ethylmalonic Encephalopathy (EE) than to Arabidopsis glyoxalase II enzymes (FIG. 2A). ETHE1 does not show kinetic activity with the glyoxalase substrate SLG. Based on these results, we have renamed the Arabidopsis gene locus At1g53580 AtETHE1. EE is found mainly in people of Mediterranean or Arabic decent, and symptoms include chronic diarrhea, a delay in neural development, symmetric brain lesions, relapsing petechiae, and orthostatic acrocyanosis, which typically lead to a premature death within the first decade of life. While the symptoms associated with EE are well characterized, there is little insight into the underlying function of huETHE1.

Sequence comparisons of At1g53580 showed high similarity to β-lactamase fold containing proteins, with the greatest overall similarity to the glyoxalase 2 family of proteins.

Within the glyoxalase II-like family of proteins, AtETHE1 shows the greatest similarity to huETHE1 enzymes. Alignment of AtETHE1 with HuEthe1 and the four Arabidopsis glyoxalase II isozymes showed that AtETHE1 is 54% identical with HuEthe1, but only exhibits 13% similarity to the glyoxalase II isozymes.

Molecular Analysis of AtETHE1

The At1g53580 cDNA was isolated and compared with the corresponding genome sequence for At1g53580 which consists of 7 exons and 6 introns (FIG. 1). The At1g53580 cDNA is capable of encoding a 256 amino acid protein that is homologous to human Ethylmalonic Encephalopathy protein 1 (huETHE1). Although At1g53580 had previously been predicted as a Glyoxalase II enzyme, it is clearly more similar to the human Ethel than the Arabidopsis GLX2 isozymes. Alignment of At1g53580 (AtETHE1) with HuEthe1 and the four Arabidopsis glyoxalase II isozymes showed that it is 54% identical with HuEthe1, but only exhibits 13% similarity to the glyoxalase II isozymes (FIGS. 2A & B). Based on these results, we have renamed the Arabidopsis At1g53580 gene locus AtETHE1.

ETHE1-like proteins are found in almost all forms of life including animals, plants, insects, eubacteria and archaebacteria. Although ETHE1 enzymes are most similar to GLX2 proteins, this similarity is significantly less than that observed between GLX2 isozymes. For example, Arabidopsis cytoplasmic GLX2 (AtGLX2-2) shows approximately 55% identity with the mitochondrial GLX2 isozymes (AtGLX2-1, 2-4 and 2-5), but only 28% identity with AtETHE1. Likewise AtGLX2-1, AtGLX2-4 and AtGLX2-5 are approximately 80% identical, but show only 26% identity with AtETHE1. Many of the conserved amino acids between ETHE1 and GLX2 enzymes are found in the β-lactamase fold and are involved in metal binding.

Therefore, while sequence comparisons indicate that ETHE1 proteins are β-lactamase fold containing proteins that are most similar to GLX2, the relatively low levels of similarity with GLX2 suggest that they do not function in the glyoxalase pathway, but rather have a unique biochemical function. Recent results from our lab, which demonstrated that AtETHE1 can not utilize SLG, GLX2 substrate, or other glutathione derivatives as substrates, support this conclusion.

Materials and Methods

Protein sequences of AtETHE1 (At1g52580, gi:79606538, genebank accession # NP_{—}974018), ETHE1 (gi:41327741, genebank accession # NP_{—}055112), GLX2-5 (gi:73621009, genebank accession # Q9SID3), and human GLX2 (gi:1237213, genebank accession # CAA62483) were obtained from the NCBI databank. The sequence alignment was created by running a structure-based alignment with VAST between AtETHE1 and GLX2-5, and then performing a pairwise alignment between AtETHE1 and ETHE1, and between GLX2-5 and human glyoxalase II.

### Example 2

### Structure of an ETHE1-Like Protein from Arabidopsis Thaliana

The protein product of gene At1g53580 from Arabidopsis thaliana possesses 54% sequence identity to a human enzyme that has been implicated in the rare disorder ethylmalonic encephalopathy. The structure of the At1g53580 protein has been solved to a nominal resolution of 1.48 Å. This structure reveals tertiary structure differences between the ETHE1-like enzyme and glyoxalase II enzymes that are likely to account for differences in reaction chemistry and multimeric state between the two types of enzymes. In addition, the Arabidopsis ETHE1 protein is used as a model to explain the significance of several mutations in the human enzyme that have been observed in patients with ethylmalonic encephalopathy.

The Arabidopsis thaliana gene At1g53580 encodes a 294-residue protein whose sequence places it in the metalloβ-lactamase superfamily (SUPFAM E value=3×10⁻¹³). This protein was originally identified as one of five glyoxalase II isozymes in Arabidopsis. Structures of two glyoxalase II enzymes are currently known. One corresponds to a cytoplasmic isozyme from Homo sapiens and the other is a mitochondrial isozyme from A. thaliana, which has been designated AtGLX2-5 (At2g31350).

Glyoxalase II (GLX2; also known as hydroxyacylglutathione hydrolase) along with glyoxalase I (GLX1) makes up the glyoxalase system that acts to convert a variety of α-keto aldehydes into hydroxyacidsin the presence of glutathione. Aromatic and aliphatic α-keto aldehydes react spontaneously with glutathione to form thiohemiacetals, which are converted to S-(2-hydroxyacyl)-glutathione derivatives by GLX1. GLX2 hydrolyzes these derivatives to regenerate glutathione and produce hydroxyacids. Glyoxalase I utilizes a number of α-ketoaldehydes. However, the primary physiological substrate of the enzyme is thought to be methylglyoxal (MG), a cytotoxic and mutagenic compound formed primarily as a byproduct of carbohydrate and lipid metabolism. Therefore, the glyoxalase system is thought to play an important role in chemical detoxification. Glyoxalase II enzymes, like other members of the metallo-β-lactamase superfamily, have been shown to contain dinuclear metal centers. Interestingly, different glyoxalase II enzymes have differing specificities for iron, zinc and #2006 International Union of Crystallography manganese.

In Example 1, the Arabidopsis gene locus At1g53580 was named AtETHE1. We will subsequently refer to the protein product of this gene as AtETHE1. While the human ETHE1 protein (referred to hereafter as huETHE1) shows significant sequence similarity to glyoxalase II, it does not possess glyoxalase II activity. No function has been determined for the enzyme; however, it has been implicated in a rare autosomal recessive disorder known as ethylmalonic encephalopathy and a number of mutations in the huETHE1 protein of affected individuals have been identified.

In this Example, we describe the structure of AtETHE1 and demonstrate the structural differences between AtETHE1 and glyoxalase II enzymes. We further illustrate the structural significance of several mutations within the huETHE1 enzyme found in sufferers of ethylmalonic encephalopathy.

Materials and Methods

Cloning, expression and purification: The AtETHE1 cDNA was cloned into pET24b as an NdeI and XhoI fragment following PCR amplification using the primers 3′-TCTTCTCATATGAAGCTTCTCTTTCGTCAAC (SEQ ID NO: 16) and 5′-GAGTCGACTCGAGCTCTAGATC (T)₁₆ (SEQ ID NO: 17). For high-level expression in Escherichia coli, the N-terminal 11 amino acids were removed and the amino-terminal methionine placed at amino acid 12 of the predicted protein sequence. After verification by DNA sequencing, pET24b-AtETHE1 was transformed into BL21-Codon Plus (DE3)-RIL cells and used for protein overexpression in ZY medium containing 50 μg ml⁻¹ kanamycin and 50 μM Fe(NH₄)₂(SO₄)₂ as described previously (Zang et al., (2001) J. Biol. Chem. 276:4788-95). AtETHE1 was purified from cleared lysates by FPLC using a Q-Sepharose column as described previously (Crowder et al., (1997) FEBS Lett. 418:351-54). Protein purity was determined by SDS-PAGE and protein concentrations were determined using the extinction coefficient 10 240 M⁻¹ cm⁻¹, which is based on the amino-acid composition of AtETHE1 (Gill & von Hippel, (1989) Anal. Biochem. 182:319-326). Metal analyses were performed on the purified enzyme using a Varian Liberty 150 inductively coupled plasma spectrometer with atomic emission spectroscopy detection (ICP-AES) as described by Crowder et al. (1997).

Crystallization:

AtETHE1 crystals were grown by the hanging-drop vapor-diffusion method at 293 K. The reservoir solution contained 24% (w/v) polyethylene glycol methyl ether 5K, 0.05 M magnesium sulfate and 0.10 M N-(2-hydroxyethyl)piperazineN′-(2-ethanesulfonic acid) (HEPES) pH 8.5. The protein solution contained 10 mg ml⁻¹ protein and 10 mM MOPS pH 7.2. Drops were produced by mixing 2 μl protein solution with 2 ml reservoir solution on a Nextal cover slip (Nextal Biotechnologies, Montreal). Two to three drops were placed on each cover slip and the cover slip was then used to seal a tray well containing 500 μl reservoir solution. The crystals were grown at room temperature and diffraction-quality crystals appeared after several months. Additional crystals used for phasing were grown within a week following micro-seeding and then soaked in a solution of mother liquor containing 2 mM thimerosal (C₉H₉HgNaO₂S) for 2 d. Crystals were then cryoprotected by soaking in solutions of mother liquor with increasing amounts of ethylene glycol up to 20% (v/v).

Data Collection:

Diffraction data from the native AtETHE1 crystal were collected at liquid-nitrogen temperatures on beamline 22-ID at Argonne National Laboratories at a wavelength of 1.23984 Å to a maximum resolution of 1.48 Å. Data were collected on a MAR 300 charge-coupled device using 2 s exposures and a frame width of 1°. Diffraction data from the mercury-derivatized crystal were collected on beamline 23ID-B at Argonne National Laboratories at a wavelength of 0.98244 Å to a maximum resolution of 2.04 Å. Data were collected on a MAR 300 charge-coupled device using 6 s exposures and a frame width of 1°. The diffraction images were integrated and scaled using HKL-2000 (Otwinowski & Minor, (1997) Methods Enzymol. 276:307-326). The overall anomalous R factor for the derivative data set as calculated by SCALEPACK was 0.062 and the overall R factor between the native and derivative data set was 0.224.

Structure Determination and Refinement:

The mercury substructure of the derivatized crystal was determined using HySS from PHENIX (Adams et al., (2002) Acta Cryst. D58:1948-54; Weeks et al., (2003) Methods Enzymol. 374:37-83), which detected four Hg atoms within the asymmetric unit. The mercury positions were input into autoSHARP to calculate phases using single isomorphous replacement with anomalous scattering phasing techniques (Bricogne et al., (2003) Acta Cryst. D59:2023-2030). Auxiliary programs used by auto-SHARP were from the CCP4 suite (Collaborative Computational Project, Number 4, 1994). Density modification was carried out with SOLOMON (Abrahams & Leslie, (1996) Acta Cryst. D52:30-42). ARP/wARP was used to build the initial model (Lamzin & Wilson, (1993) Acta Cryst. D49:129-147). The model was completed with alternate rounds of model building with Coot (Emsley & Cowtan, (2004) Acta Cryst. D60:2126-2132) and restrained refinement via REFMAC (Murshudov et al., (1997) Acta Cryst. D53:240-255).

The final model contained four protein molecules, four iron(II) ions, 1037 water molecules, 14 ethylene glycol molecules and a sulfate molecule. Eight of the C-terminal residues for two of the protein chains and the final C-terminal residue for the other two chains were left unmodeled. The discrepancy in the observable length of the four chains arose from additional non-biological contacts that the C-terminal regions of chains A and C were able to make with chains D and B. The Ramachandran plot showed that 92% of the residues were in the most favorable region. The remainder were in the generously allowed region of the plot. The data-collection and refinement statistics are summarized in Table 2.

TABLE 2
Summary of crystallographic data-collection and refinement statistics.
(Values in parentheses refer to the highest resolution shell.)

| Data set | Native | Hg derivative |
|---|---|---|
| Data collection | | |
| Wavelength (A°) | 1.23984 | 0.98244 |
| Resolution range (A°) | 46.11-1.48 | 49.11-2.04 |
| | (1.51-1.48) | (2.09-2.04) |
| Space group | P2₁ | P2₁ |
| Unit-cell parameters | | |
| a (Å) | 66.6 | |
| b (Å) | 64.5 | |
| c (Å) | 127.9 | |
| β (°) | 97.8 | |
| Measured/unique reflections | 1005815/170395 | 448871/66747 |
| Completeness (%) | 94.6 (67.7) | 97.0 (73.9) |
| R_{merge} | 0.075 (0.435) | 0.128 (0.364) |
| Average I/σ(I) | 12.43 (2.82) | 8.07 (2.29) |
| Redundancy | 5.9 (2.5) | 6.7 (3.5) |
| Phasing | | |
| Mean FOM (centric/acentric) | | 0.29966/0.31622 |
| Isomorphous R_{cullis} | | 0.851/0.850 |
| (centric/acentric) | | |
| Anomalous R_{cullis} | | 0.919 |
| Refinement statistics | | |
| R_{cryst}/R_{free} (%) | 17.7/20.4 | |
| Ramachandran plot | | |
| Most favorable regions (%) | 92.0 | |
| Additionally allowed regions (%) | 8.0 | |
| Generously allowed regions (%) | 0.0 | |
| Disallowed regions (%) | 0.0 | |
| R.m.s. deviations from ideality | | |
| Bonds (Å) | 0.015 | |
| Angles (Å) | 1.636 | |
| Average B value (Å²) | 19.4 | |
| Average protein B value (Å²) | 17.5 | |
| Average solvent B value (Å²) | 32.5 | |

Results and Discussion

Overall Fold:

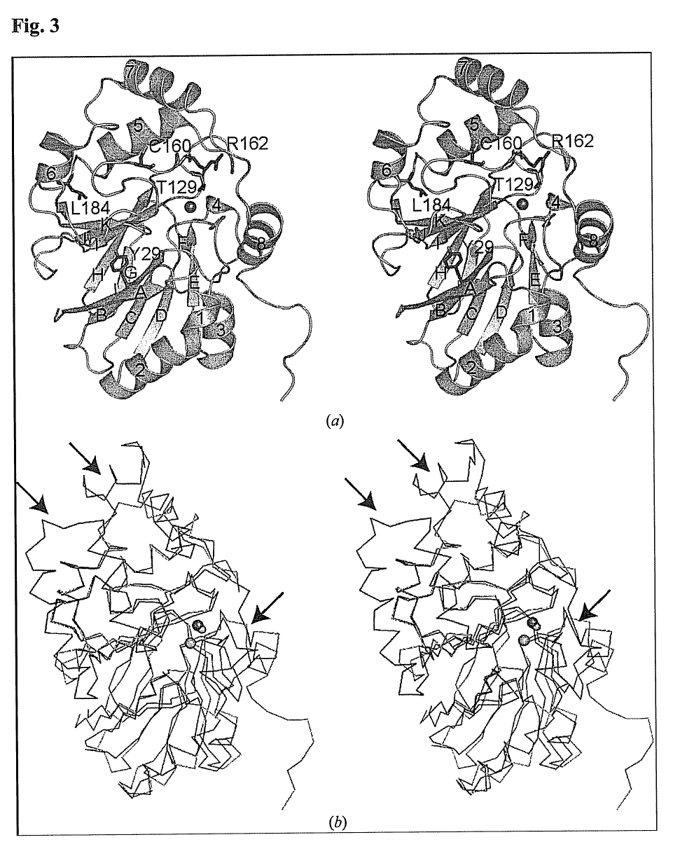

The overall fold of AtETHE1 is typical of the β-lactamase superfamily. It contains two central mixed β-sheets, each containing six strands, surrounded on both sides by helices (see FIG. 3). The β-sheet topology is of the order A, B, C, D, E, F and G, H, I, J, K, L. β-Strands A, B and C are aligned anti-parallel, whereas C, D, E and F are parallel. In the second β-sheet, strands G, H, I and J are aligned antiparallel, J and K are aligned parallel and K and L are aligned anti-parallel.

A VAST search indicated that the most structurally similar enzymes are the human and Arabidopsis (AtGLX2-5) glyoxalase II enzymes, with scores of 27.7 and 27.2, respectively. The overall folds of AtETHE1 and AtGLX2-5 are highly similar, but differ in three regions, as shown in FIG. 3. The first two regions are outside the active site but make contacts with one another. This includes a two-helix bundle that extends from residues 172 to 206 and an extended loop consisting of residues 223-240 in AtGLX2-5. Both of these features are missing in AtETHE1. Additionally, the extended C-terminus of AtETHE1 reaches across the opening of the active site, greatly limiting the possible size of potential substrates. These gaps are further illustrated in FIG. 2B.

Dimer:

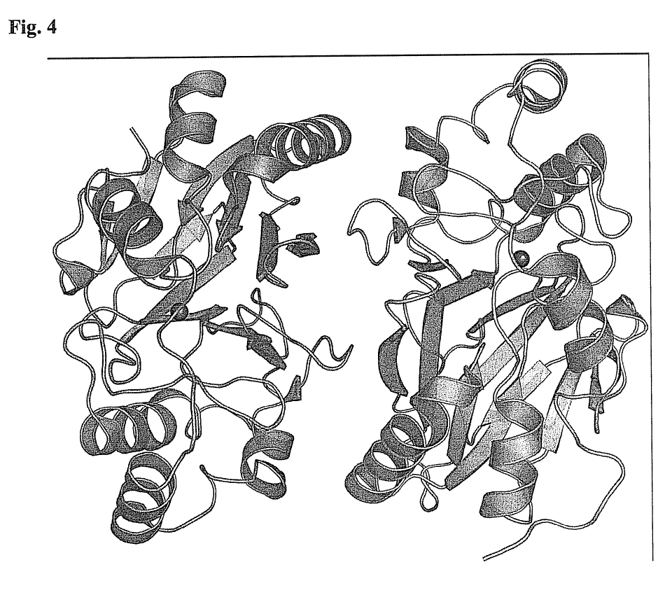

While previously described glyoxalase II enzymes are monomers, the crystal structure of AtETHE1 reveals a dimeric organization for the protein (see FIG. 4). The human ETHE1 protein was shown to be a dimer by gel-filtration chromatography. Interestingly, the dimerization interface for AtETHE1 appears to be in a region that was blocked by the two-helix bundle of the AtGLX2-5 enzyme. This may represent a distinguishing feature between ETHE1-like and glyoxalase II enzymes. The interface between the AtETHE1 dimers is not extensive, with only 830 Å² of buried surface area. The interface contains 58% nonpolar area and involves ten residue-to-residue hydrogen bonds. The interactions are identical in the two subunits, with Arg17 forming hydrogen bonds with Glu206 from the other subunit and likewise Gln18 with Glu200, Phe20 with Gly198, Arg53 with Lys197, Glu60 with Arg158 and vice versa.

Metal-Binding Site:

Only one metal atom was located within the electron density for AtETHE1. ICP-AES results indicated that the purified enzyme contained two molar equivalents of iron; however, ICP-MS metal analysis of the protein after being subjected to freezing and storage gave a metal/protein ratio of 0.56. This closely matches what was observed in the crystal structure, where an occupancy of 0.5 for the iron ion gave the best refinement results. Despite this partial occupancy, the electron density for the coordinating ligands is well defined without any indication of heterogeneity or multiple conformers.

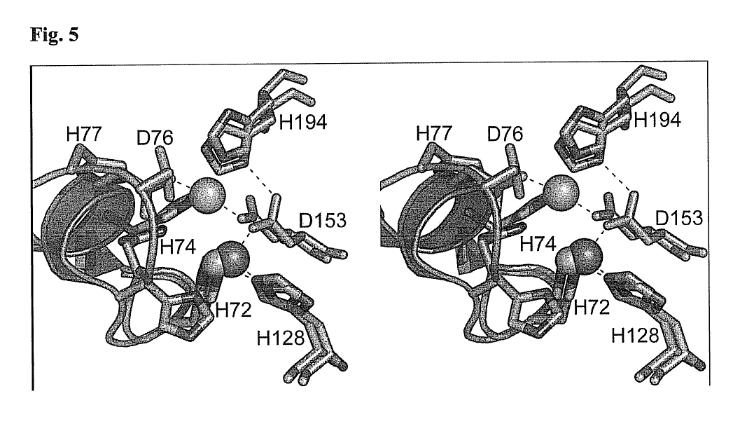

The iron ion location is identical to one of the two metal ions in the AtGLX2-5 protein structure. FIG. 5 depicts an overlay of the residues involved in metal binding in the AtGLX2-5 and AtETHE1 enzymes. In the AtGLX2-5 protein structure, one metal ion was tetrahedrally coordinated to three histidines (His54, His56 and His112) and a bridging water molecule. The coordination of the equivalent iron ion in the AtETHE1 structure was octahedral. In addition to three waters, the iron ion was bound to His72 and His128, the homologs of His54 and His112 in AtGLX2-5. The homolog of Asp131, Asp153, which binds the second metal atom in AtGLX2-5, is slightly shifted such that it also directly coordinates the iron ion in AtETHE1. The homolog of His56, His74, does not coordinate the metal in AtETHE1.

In the AtGLX2-5 structure there is a single-turn helix containing His56 near its N-terminal end. In the AtETHE1 structure, this helix has been pulled apart such that the side chain of His74 is no longer directed towards the metal atom. This change also displaces the side chains of Asp76 and His77, whose structural equivalents in AtGLX2-5 coordinate the second metal. It is unclear if the unwinding of this helix simply arises from a missing metal atom or if it accurately represents an active conformation of the protein. There are two sequence features of AtETHE1 that indicate this unraveling may be more likely to occur in this enzyme than in the glyoxylase II isozymes. The unwinding of this helix places the side chain of Ala75 directly into the active site of the enzyme. In glyoxylase II enzymes, this alanine is replaced by a residue with a bulky side chain, for instance a tyrosine in AtGLX2-5, which may cause additional steric problems upon unwinding of the helix. In addition, there is a modification to a conserved glyoxylase II CGK(L/F)(F/Y)EG (SEQ ID NO: 18) motif (Cys138, Gly139, Lys140, Leu141, Phe142, Glu143 and Gly144 in AtETHE1) which alters the sequence to CGRTDFQEG (SEQ ID NO: 19). The side chain of the inserted glutamine, Gln166, directly occupies the space where the side chain of His74 would need to be to coordinate the Fe ion and fowls a hydrogen bond with the carbonyl of Val73. This displacement is further stabilized by hydrogen bonds formed between the side chains of Asp164 and Arg162. These residues appear to be strongly conserved in ETHE1 proteins.

The second metal atom in AtGLX2-5 was coordinated by His59, His169, Asp58, Asp131 and a bridging water molecule. As mentioned previously, the homologs of His59 and Asp58 in AtETHE1 are displaced owing to the unraveling of a single-turn helix; however, the homolog of Asp58 (Asp76) is still within coordination distance of a second metal atom. The homolog of His59 (His77) is pulled away from the putative second metal atom site and in its current position is unlikely to coordinate a metal atom. The side chain of His74 in the AtETHE1 enzyme is near the location His77 would need to occupy if it were to coordinate a second metal atom and may serve as a replacement for binding a second metal atom. In the AtETHE1 structure a carboxyl O atom of the homolog of Asp131 is 2.1 Å from the iron ion and also near the expected location of the second metal atom. The homolog of His169, His194, is positioned identically to His169 and could conceivably coordinate a second metal atom in AtETHE1. Ultimately, all of the residues necessary for binding a second metal atom similarly to GLX2-5 are present in AtETHE1; however, some structural rearrangements would have to occur to obtain the necessary orientation of the metal-binding side chains.

The metal and potential substrate-binding residues in AtETHE1 are for the most part involved in the same hydrogen-bonding networks observed in AtGLX2-5. The primary exception is His194, which does not coordinate a metal in the AtETHE1 structure. The equivalent histidine in AtGLX2-5 was stabilized by interactions with the carboxyl O atom of an aspartate side chain. The remaining carboxyl O atom was hydrogen bonded to a lysine side-chain N atom. In AtETHE1, this aspartate is replaced with a serine, limiting further electron delocalization. Also, the imidazole side chain of His194 is flipped in the AtETHE1 structure and the ND1 N atom instead interacts with the side chain of Asp153.

Active Site:

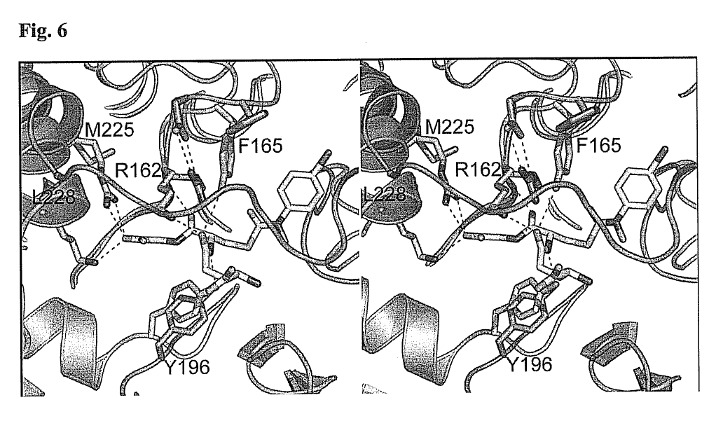

Residues involved in substrate binding in the human glyoxalase II are almost entirely conserved in AtGLX2-5. This is not the case with AtETHE1. An overlay of the human glyoxalase II substrate-binding site with the equivalent residues in AtETHE1 is depicted in FIG. 6. The side chains of residues Arg249 and Lys252 make hydrogen bonds with the glycine portion of glutathione in the human enzyme. These residues are replaced with Met225 and Leu228 in AtETHE1. Leu228 is also pulled away from the substrate-binding region. The backbone amino group of Lys143 and the side chain of Tyr175 in human glyoxalase II were shown to hydrogen bond to the cysteine portion of glutathione. In AtETHE1, Lys143 is replaced by Arg162; however, the backbone amino groups of these two residues overlap. Replacement of this lysine with arginine appears to be common among ETHE1 proteins and loss of this arginine has been linked with ethylmalonic encephalopathy in humans. The AtETHE1 homolog of Tyr175, Tyr196, is also positioned similarly to that of the human enzyme. In human glyoxalase II the side chains of Tyr145 and Lys143 form hydrogen bonds with the glutamate portion of the glutathione. AtETHE1 has a phenylalanine in place of Tyr145; however, Arabidopsis glyoxalase II AtGLX2-5 has the same substitution. Lys143 is replaced by Arg162 in AtETHE1. The arginine side chain could conceivably also interact with the substrate. The side chain of Arg162 appears to be firmly held in place by two hydrogen bonds with the side-chain carboxyl of Asp164 and is not likely to be as flexible as Lys143.

The active site of AtETHE1 has significantly less room for substrate binding than that of the human and Arabidopsis glyoxalase II enzymes owing to the unwound helix described above and the extended C-terminal region that covers up much of the active site. While there appears to be enough room to fit a glutathione group, there is substantially less space available for the other portion of the thioester. The other subunit of the dimer does not appear to alter the active site.

Structural Basis for Encephalopathy:

A number of single-residue mutations have been identified in the ETHE1 protein of patients with ethylmalonic encephalopathy and a model of human ETHE1 was created based on the human glyoxalase II crystal structure (Tiranti et al., (2006) Am. J. Hum. Genet. 74:239-252). Our results confirm many of the observations of this study and provide some additional details (FIG. 3). Mutations in human ETHE1 include R163Q, C161Y, Y38C, L185R and T136A. The Arabidopsis equivalent of Arg163, Arg162, is located within the expected active site of the enzyme and forms two hydrogen bonds with the side chain of Asp164. Given the location of Arg162, it seems likely that this residue may be directly involved in the catalytic mechanism of the enzyme. The Arabidopsis equivalent of Cys161, Cys160, is also near the active site. Mutation of this residue into a bulky tyrosine would clearly reposition Arg162 and possibly other amino acids involved in substrate binding. The Arabidopsis equivalent of Thr136, Thr129, forms a hydrogen bond with a backbone amine N atom that may help stabilize His128, which coordinates the iron ion in AtETHE1. The Arabidopsis equivalent of Tyr38, Tyr29, is part of the hydrophobic interface between the two internal, B-sheets and sits in a pocket of cyclic aromatic side chains which include Phe27, Phe16, Phe156 and Tyr191. In addition, the Tyr29 hydroxyl group forms a hydrogen bond with the side chain of Gln18, which is part of the dimer interface. Mutation of this residue could subsequently have repercussions on the stability of both the tertiary and quaternary structures. The Arabidopsis equivalent of the final relevant human residue, Leu184, is far from the active site and sits in the region between the C-terminal β-sheet and the α-terminal ct-helical region. The residue is near the side chains of Arg123 and Arg147. Mutation of this residue to another arginine would be likely to be highly destabilizing.

Sequence Analysis:

A series of BLAST searches were conducted to determine the prevalence of ETHE1-like enzymes. The assumption was made that the presence of Arg162, the absence of residues involved in binding glutathione and the absence of the two-helix bundle observed in glyoxalase II are features that distinguish between ETHE1 and glyoxalase II enzymes. ETHE1-like enzymes have been observed in almost all forms of life, including animals, plants, fungi, eubacteria and archaebacteria. Unlike glyoxalase II enzymes, multiple isozymes of the ETHE1 have not been detected within a species. It was also noted that in some archaebacteria, such as halobacteria, the ETHE1-like fold was coupled to a rhodanese transferase-like domain.

Conclusions:

The crystal structure of AtETHE1 has been solved and refined to 1.48 Å. The structure reveals a fold that varies from the closely related enzyme glyoxalase II. The removal of a two-helix bundle in AtETHE1 results in the formation of a dimer interface that is missing from the glyoxalase II enzymes. The extended C-terminus which aligns the active site, as well as several changes in the substrate-binding residues of glyoxalase II enzymes, allow a different and unknown reaction chemistry. The structure also revealed a metal-binding site as well as a possible second metal site given some structural rearrangement. In addition, the structure of AtETHE1 is the closest model available for human ETHE1 and provides a structural explanation for the deleterious effects of several mutations corresponding to the onset of ethylmalonic encephalopathy as well as revealing the active-site architecture involved in binding an as yet unknown substrate.

### Example 3

### Spectroscopic Studies on Arabidopsis ETHE1, a Glyoxalase II-Like Protein

In spite of the apparent importance of the ETHE1 enzyme, very little is known about its function or biochemical properties. In this Example, Arabidopsis ETHE1 was over-expressed and purified and shown to bind tightly to 1.2±0.2 equivalents of iron. ¹H NMR and EPR studies demonstrate that the predominant oxidation state of Fe in AtETHE1 is Fe(II), and NMR studies confirm that two histidines are bound to Fe(II). EPR studies show that there is no antiferromagnetically-coupled Fe(III)Fe(II) center in AtETHE1. Gel filtration studies reveal that AtETHE1 is a dimer in solution, which is consistent with previous crystallographic studies. Although similar in terms of amino acid sequence to glyoxalase II, AtETHE1 exhibits no thioester hydrolase activity, and activity screening assays reveal that AtETHE1 exhibits low level esterase activity. Taken together, AtETHE1 is a novel, mononuclear Fe(II)-containing member of the β-lactamase fold superfamily.

ETHE1-like enzymes are found in most organisms, suggesting that it serves a fundamental biochemical role in nature; however the exact biochemical role of ETHE1 is currently unknown. ETHE1 shows the greatest sequence similarity to the glyoxalase II (GLX2) family of proteins.

GLX2 enzymes belong to the metallo-β-lactamase fold family of proteins, which are typically dinuclear metallohydrolases. The metallo-β-lactamases typically bind two equivalents of Zn(II) and hydrolyze β-lactams. GLX2 enzymes, which hydrolyze SLG, contain dinuclear metal centers that can bind Zn, Fe, and Mn. Additional protein families containing the β-lactamase fold include the rubredoxin:oxygen oxidoredutase (ROO) and ZiPD families, which have been shown to bind divalent Fe and Zn(II), respectively. Therefore, the β-lactamase fold can accommodate several different metals and is present in enzymes that can catalyze a wide range of reactions. The GLX2 dinuclear metal binding center typically consists of one metal site that is tetrahedrally-coordinated by three histidines and a bridging hydroxide, while the second metal site is coordinated by two histidines, two aspartates, a bridging hydroxide, and one terminally-bound water.

Arabidopsis ETHE1 is 54% identical to human ETHE1. Crystal structure comparisons between Arabidopsis ETHE1, which was previously characterized as a glyoxalase enzyme, and Arabidopsis GLX2-5, a mitochondrial GLX2 enzyme, revealed that while the proteins show only 13% sequence identity, AtETHE1 is structurally very similar to GLX2 (Example 2). ETHE1 enzymes share several structural features with GLX2 enzymes, including sequence and structural characteristics of the metal binding domain and the β-lactamase fold consisting of two central mixed β-sheets surrounded on both sides by helices. Interestingly, even though ETHE1 and GLX2 enzymes exhibit extensive similarity in their metal binding regions, the AtETHE1 crystal structure showed it only bound a single metal atom. In fact, the crystal structure was best refined to a metal occupancy of 0.5 (Example 2). However, the metal content of purified AtETHE1 was reported to be 2 equivalents of metal per equivalent of protein, raising the question of the actual metal content of this enzyme.

Several other features have been identified that set AtETHE1 apart from the GLX2 family. An important difference between the two proteins involves the substrate binding pocket. Sequence alignments, as well as the crystal structure comparison, identified several amino acid substitutions in AtETHE1 of residues involved in SLG binding in GLX2 (Example 2). These changes in addition to the presence of an extended C-terminal tail are predicted to render the substrate binding pocket of AtETHE1 too small for the GLX2 substrate SLG. Additionally, unlike GLX2, AtETHE1 was predicted to function as a dimer due to the absence of a two helix bundle in AtETHE1 that is present in the GLX2 enzymes (Example 2).

We have over-expressed, purified, and biochemically- and spectroscopically-characterized Arabidopsis ETHE1 to investigate these differences further and to better understand the functional role of ETHE1. The results of these studies show that AtETHE1 is homodimeric in solution, exhibits low-level esterase activity, and specifically binds a single Fe(II) atom in the active site.

Materials and Methods

Over-Expression and Purification of AtETHE1.

The Arabidopsis ETHE1 cDNA was obtained from Arabidopsis bud RNA and cloned into pET24b as NdeI and XhoI fragments following reverse transcription and PCR amplification using the primers GLX2-3 (TCTTCTCATATGAAGCTTCTCTTTCGTCAAC) (SEQ ID NO: 16) and a 3′ poly (A) anchor primer. During cloning the N-terminal leader peptide was removed for high-level expression of AtETHE1 in E. coli, resulting in the amino terminal methionine, which corresponds to amino acid 50 of the predicted protein sequence. This is the same form of the protein that was used for crystal structure determination (Example 2). After verification by DNA sequencing, pET24b-AtETHE1 was transformed into BL21-Codon Plus (DE3)-RIL cells and used for protein over-expression in ZY medium as previously described in T. M. Zang, et al., J. Biol. Chem. 276 (2001) 4788-4795. AtETHE1 was purified from cleared lysates by Fast Performance Liquid Chromatography (FPLC) using a Q-Sepharose column as described previously (M. W. Crowder, et al., FEBS Lett. 418 (1997) 351-354). Protein purity was determined by SDS-polyacrylamide gel electrophoresis, and protein concentrations were determined by using an extinction coefficient (ε₂₈₀ₙₘ) of 7,240 M⁻¹cm⁻¹, which was determined using amino acid analyses.

Metal Analyses.

Metal analyses were performed on the purified enzyme using a Varian-Liberty 150 inductively coupled plasma spectrometer with atomic emission spectroscopy detection (ICP-AES) as described previously (A. D. Cameron, et al., Structure 7 (1999) 1067-1078). The purified protein was diluted to 10 μM in 50 mM TRIS, pH 7.2, and analyzed for the presence of zinc, manganese, iron, and copper. The data presented in this report represent the average of at least three preparations for each metal addition experiment.

Native Molecular Weight Determination.

The native molecular weight of AtETHE1 was determined by utilizing a Sephacryl 5200 column in 10 mM MOPS, pH 7.2, containing 0.15 M NaCl. AtETHE1 (1 mg), purified as described above, was mixed with 1 mg of each of the protein standards: Blue Dextran, bovine serum albumin, ovalbumin, aldolase, and ribonuclease A. One milliliter fractions were collected with a flow rate of 0.5 ml/min, and samples containing protein were identified by monitoring A₂₈₀ and by SDS-PAGE gel analysis.

Substrate Analysis.

Enzymatic assays were conducted at 25° C. in 10 mM MOPS, pH 7.2, on a Cary IE UV-Vis spectrophotometer. A series of thioesters of glutathione were synthesized as previously described (L. Uotila, Meth. Enzymol. 77 (1981) 424-430), and all other substrates were purchased commercially. The hydrolysis of S-D-lactoylglutathione (Sigma), S-D-acetylglutathione, S-D-acetoacetylglutathione, S-D-formylglutathione, S-D-glyocosylgutathione or S-D-pyruvylglutathione was monitored at 240 nm. S-D-mandeloylglutathione hydrolysis was monitored at 263 nm (L. Uotila, Meth. Enzymol. 77 (1981) 424-430). Hydrolysis of p-nitrophenyl phosphate (Sigma), p-nitrophenyl sulfate (Sigma), and p-nitrophenyl acetate (Sigma) was monitored at 400 nm (see R. A. Anderson, et al., Proc. Natl. Acad. Sci. 72 (1975) 2989-2993; J. J. Brandt, et al., Anal. Biochem. 272 (1999) 94-99; J. M. Armstrong, et al., J. Biol. Chem. 241 (1966) 5137-5149). The hydrolysis of L-alanine-p-nitroanilide (Sigma) was measured at 404 nm (J. J. Brandt, et al., Anal. Biochem. 272 (1999) 94-99). Ala-ala-ala-p-nitroanilide (Sigma) and y-L-Glu-p-nitroanalide (Sigma) hydrolysis was monitored at 410 nm. Hydrolysis of benzoylglycyl-L-phenylalanine was monitored at 254 nm (J. F. Sabastian, et al., Can. J. Biochem. 56 (1978) 329-333). Nitrocefin (Becton-Dickinson) hydrolysis was monitored at 485 nm (M. W. Crowder, et al., Biochemistry 35 (1996) 12126-12132). Methylglyoxal was assayed colorimetrically by using the 2,3-dinitrophenylhydrazine-alkali reaction (R. A. Cooper (1975) in Meth. Enzymol. (Abelson, J. N., and Simon, M. I., Eds.), pp. 502-508, Academic Press, New York). Assays were performed for 5 min using 80 μM substrate and varying concentrations of pure enzyme both as-isolated and after loading with excess iron.

Additional substrate screening was performed using the Micronaut-Taxa Profile E (Merlin GmbH) microtiter plate (A. Vogel, et al., Biochem. Biophys. 401 (2002) 164-172). Each well was filled with 25 μl of 40 μM AtETHE1, and reactions prepared according to manufacturer's directions. A negative control was performed using MOPS, pH 7.2, to rule out non-specific reactions. The reactions were monitored visually for 24 hours at 37° C., and positive reactions were recorded. Each reaction was performed in duplicate using enzyme loaded with iron.

EPR Spectroscopy.

EPR spectra were obtained at 9.63 GHz and 10 K using a Bruker EleXsys E580 spectrometer equipped with an ER4116DM cavity, and an Oxford ESR900 liquid helium flow cryostat and ITC503 temperature controller. Acquisition parameters included 12 G (1.2 mT) field modulation at 100 kHz. Samples contained 1.6 mM AtETHE1 protein in 50 mM TRIS, pH 7.2.

¹H NMR Spectroscopy.

NMR spectra were collected on a Bruker Avance 500 spectrometer operating at 500.13 MHz, 298 K, and a magnetic field of 11.7 tesla, recycle delay (AQ), 41 ms, and sweep width, 400 ppm. Concentrated samples of AtETHE1 (1.4 mM) contained 10% D₂O for locking or 90% D₂O for monitoring of solvent-exchangeable peaks. Protein chemical shifts were calibrated by assigning the H₂O signal a value of 4.70 ppm, and a modified presaturation pulse sequence (zgpr) was used to suppress the proton signals that originated from the water molecules.

Results

Over-Expression, Purification, and Characterization of Arabidopsis ETHE1.

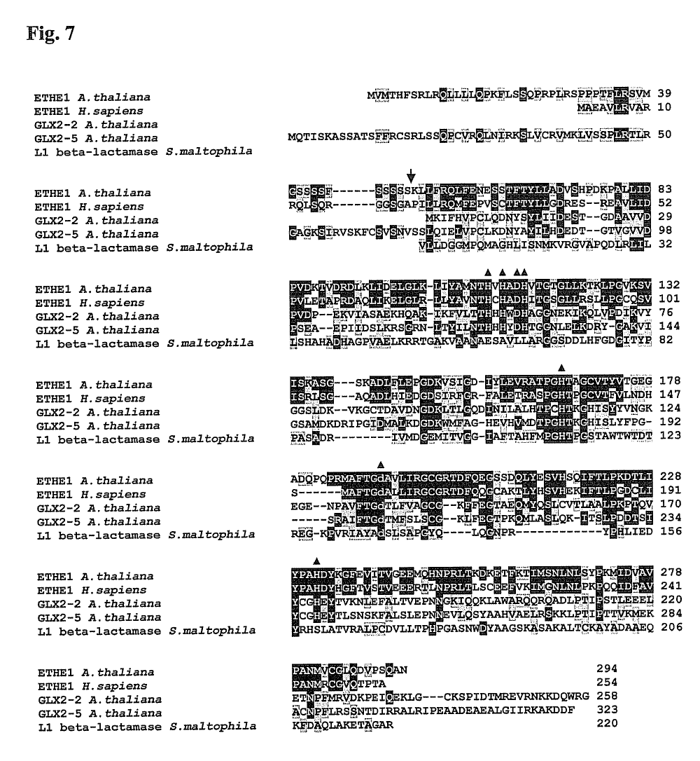

Based on publicly-available localization prediction programs (pSORT II, Mitoprot), recombinant AtETHE1 was cloned into the pET24b expression vector after removing the predicted 50 amino acid N-terminal leader sequence, which generated an N-terminus of MKLLFRQ (SEQ ID NO: 20) (FIG. 7). This plasmid was transformed into E. coli BL21(RIL) Rosetta cells, and AtETHE1 was over-expressed as described in Material and Methods. AtETHE1 was purified using FPLC Q-Sepharose chromatography, eluting from the column at ˜125 mM NaCl. Purified AtETHE1 protein was obtained in high yield (˜400-600 mg of protein/L) and was >95% pure (data not shown).

AtETHE1 is a Dimer in Solution.

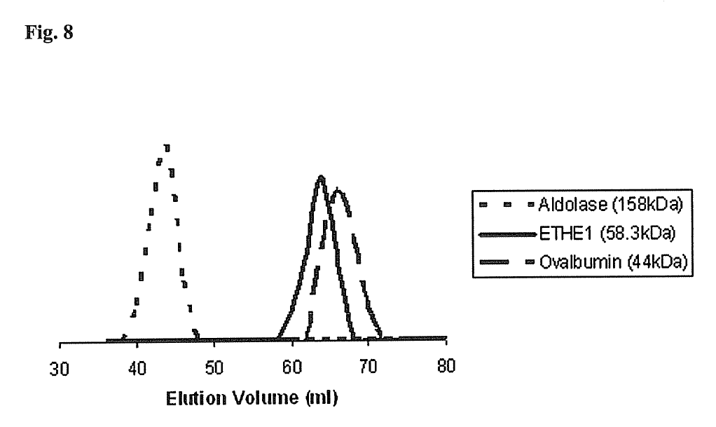

In contrast to the GLX2 enzymes, which exist as monomers in solution, the crystal structure of Arabidopsis ETHE1 suggested that it has a dimeric quaternary structure (Example 2). Gel filtration studies were performed on the recombinant AtETHE1 enzyme to test this hypothesis. AtETHE1 eluted from a Sephacryl 5200 column between standard proteins adolase (158 kDa) and ovalbumin (44 kDa), resulting in a calculated molecular weight of 58.3 kDa, which is roughly twice the recombinant monomeric weight of 26.8 kDa (FIG. 8). Therefore, Arabidopsis ETHE1 exists as a dimer in solution.

Metal Analyses.

GLX2 enzymes have been shown to bind iron, zinc, and manganese; therefore, AtETHE1 was analyzed for all three of these metals and also copper. When grown and over-expressed in rich medium containing 250 μM Fe(NH₄)₂(SO₄)₂ and Zn(SO₄)₂, AtETHE1 was found to contain 0.33±0.10 equivalents of iron and less than 0.003 eq. of zinc, manganese, and copper. While low, this result suggested that AtETHE1 may preferentially bind iron. The purified enzyme was incubated with a 4-molar excess of Fe(II) and dialyzed extensively (4×μL for four hours each) to remove loosely- or unbound Fe, and AtETHE1 was found to bind 1.2±0.2 equivalents of iron, and no detectable traces of zinc, manganese, or copper. This result is consistent with the crystal structure (Example 2), which showed that when prepared this way, AtETHE1 only binds 1 equivalent of iron. Unlike the GLX2 enzymes, the addition of excess zinc to the isolated enzyme resulted in no additional metal binding, suggesting that AtETHE1 may be specific for iron.

AtETHE1 does not Hydrolyze SLG.

AtETHE1 had previously been predicted to be a GLX2-like enzyme (M. K. Maiti, et al., Plant Mol. Biol. 35 (1997) 471-481). However, a careful sequence comparison revealed that AtETHE1 is lacking several highly-conserved amino acids known to participate in the hydrogen bonding of SLG in GLX2 (Example 2). Furthermore, our crystallographic analysis of AtETHE1 showed that the substrate binding pocket is too small to accommodate SLG. These results suggested that AtETHE1 does not utilize SLG as a substrate. AtETHE1, both as-isolated and after incubation with excess iron, was assayed for thioesterase activity with SLG and various other thioester derivatives of glutathione to test this hypothesis. Consistent with the small substrate binding pocket and absence of critical SLG binding residues, purified AtETHE1 did not hydrolyze any of the glutathione thioesters.

As-isolated AtETHE1 and Fe-enriched AtETHE1 were also assayed against 188 different substrates, including 95 substrates for peptidases, 17 substrates for diverse reactions, and 76 substrates for glycolytic enzymes, phosphatases, and esterases using a commercially-available substrate screening plate (A. Vogel, et al., Biochem. Biophys. 401 (2002) 164-172) to investigate potential substrates. Three potential substrates were identified through this screening process: Ala-Ala-Ala-p-nitroanilide (NA), Glu-pNA, and p-nitrophenyl acetate (NPA). Steady-state kinetic studies were then conducted using the three compounds. Upon further characterization, Ala-Ala-Ala-pNA and Glu-pNA were found not to be substrates for AtETHE1. However, AtETHE1 did exhibit a low level of activity against p-NPA (2.02±0.46 nmols/min/mg of enzyme). This low level of activity is similar to the esterase activity (25.8 nmols/min/mg) observed in recombinant rat carbonic anhydrase III when reacted with p-nitrophenyl acetate (G. Kim, et al., Arch. Biochem. Biophys. 377 (2000) 334-340). The activity of AtETHE1 towards p-nitrophenyl acetate was inhibited by the presence of a metal chelator, 1,10-o-phenanthroline, indicating that p-nitrophenyl acetate hydrolysis by AtETHE1 requires bound metal. Therefore, based on these results and those of the crystal structure, we predict that the AtETHE1 substrate may be a relatively small ester.

Spectroscopic Studies on AtETHE1.

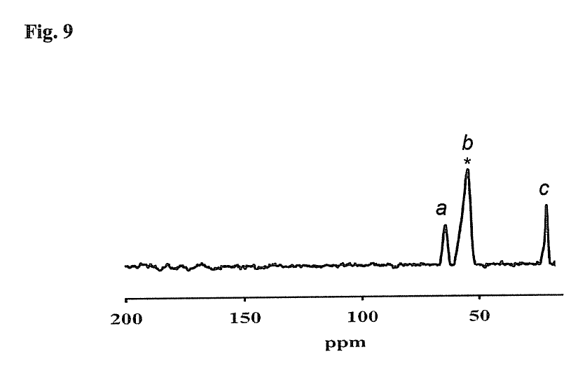

The metal binding site of iron-bound AtETHE1 was investigated using ¹H NMR spectroscopy (FIG. 9). The spectra revealed the presence of at least 4 paramagnetically-shifted resonances between 110 and −30 ppm. In this spectrum peaks a and c integrate to 1 proton each, while peak b integrates to 3 protons (FIG. 9). To further investigate the identity of these protons, solvent-exchangeable peaks were monitored in the presence of D₂O (FIG. 9). Two of the three protons were found to be solvent-exchangeable (Peak b). Based on the resonance positions and line widths, the exchangeable peaks can be assigned to the protons bound to the N—H protons on histidines bound to Fe(II) or possibly to an antiferromagnetically-coupled Fe(III)Fe(II) center (I. Bertini, et al., Chem. Rev. 93 (1993) 2833-2932; J. Moran-Barrio, et al., J. Biol. Chem. 282 (2007) 18286-18293). The AtETHE1 crystal structure shows that both of the histidine ligands are bound through the c nitrogen, and therefore, peak c and the non-exchangeable proton from peak b are likely due to the β-CH₂ protons on the metal bound histidines rather than to meta protons on the histidine ring (FIG. 9) (Example 2). This is surprising because the β-CH₂ protons do not normally shift out to these positions. We cannot rule out the possibility that these peaks are due to ortho protons on metal bound histidines, but these peaks are usually too broad to detect (Z. Wang, et al., Biochemistry 31 (1992) 5263-5268). Peak a is likely due to the β-CH₂ protons on the bound Asp ligand (FIG. 9) (Example 2). This result was surprising, since we initially predicted that AtETHE1 would have a metal binding site similar to those in the GLX2 family. NMR spectra from GLX2-5, which contains a dinuclear iron center, shows at least eight paramagnetically-shifted resonances in between 110 and −30 ppm that correspond to protons on ligands bound to a Fe(III)Fe(II) antiferromagnetically-coupled center (G. P. K. Marasinghe, et al., J. Biol. Chem. 280 (2005) 40668-40675). Our recent discovery of the crystal structure of AtETHE1 demonstrated that His 232 may not be in a position to coordinate a bound metal ion, which is consistent with NMR spectrum of AtETHE1.

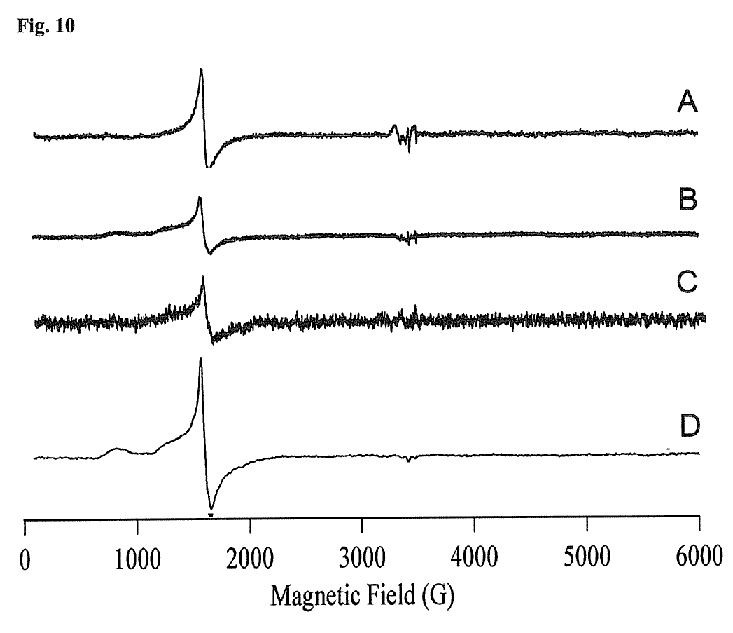

EPR spectra on several different forms of AtETHE1 were obtained to further investigate the AtETHE1 metal center. The spectrum of as-isolated AtETHE1 containing 0.33 equivalents of Fe (‘Feᵢₛₒ-AtETHE1’) indicated the presence of rhombic Fe(III) (FIG. 10). The signal was dominated by a broad derivative feature at g_{eff}=4.27; no features that could be considered diagnostic for protein-bound Fe(III) were observed and the origin of the signal is, therefore, unclear. Although precise quantitation of high-spin systems for which more than one Kramers' doublet is populated is not trivial, double integration and comparison with a similar EPR signal due to Fe(III) from a well-characterized (4-hydroxyphenyl) pyruvate dioxygenase (V. M. Purpero, et al., Biochemistry 45 (2006) 6044-6055) indicated an Fe(III) content of about 0.025 mM. Therefore about 95% of the iron in Feᵢₛₒ-AtETHE1 is EPR-silent.

An Fe(III) signal with a resonance at g ˜4.3 and additional absorption features at lower field were observed from the analysis of AtETHE1 enzyme containing 1 equivalent of Fe (Fe₁-AtETHE1) (FIG. 10B). In this spectrum, the resonance positions indicated a dominant zero-field splitting term (i.e., D,E>>gβBS; the lower field resonances centered at g ˜5.5 and g˜9 terminate abruptly at g=6 and g=10, respectively); the lack of resolved structure and the broad absorption from g˜4.3 to g˜10 indicate a fairly broad distribution of the rhombic zero-field splitting parameter (i.e., strains in E/D) and thermal population of at least two Kramers' doublets (A. J. Copik, et al., Inorg. Chem. 44 (2005) 1160-1162). Again, the signal was not definitive for site-specific binding of Fe(III) by AtETHE1, although the form of the signal, particularly the inflection on the g˜4.3 crossover due to incomplete rhombicity (i.e., E/D is slightly less than ⅓), is highly reminiscent of the spectrum due to Fe(III) bound to the active site of the metallo-β-lactamase GOB (J. Moran-Barrio, et al., J. Biol. Chem. 282 (2007) 18286-18293). The intensity of the signal was somewhat lower than that of the as-isolated AtETHE1 and accounted for <1% of the total iron. Interestingly, there was no evidence for a ‘g˜1.7, 1.8, 1.9’ signal due to an Fe(II)-Fe(III) center.

We attempted to disrupt the protein structure of Fe₁-AtETHE1 by applying freeze-thaw cycles, but the EPR signal remained relatively constant (FIG. 10C; fewer scans were averaged). The intensity of the signal was found to increase by a factor of 4 upon aerobic incubation for 100 h at 4° C. (FIG. 10D). The form of this spectrum is clearly distinct from that of Feᵢₛₒ-AtETHE1, but still accounts for <5% of the total Fe. Because of a weak background signal due to trace Mn(II) and Cu(II), the possibility of the presence of a signal due to an Fe(II)-Fe(III) center cannot be ruled out completely, but the population of such a center, if not zero, must be very low.

Discussion

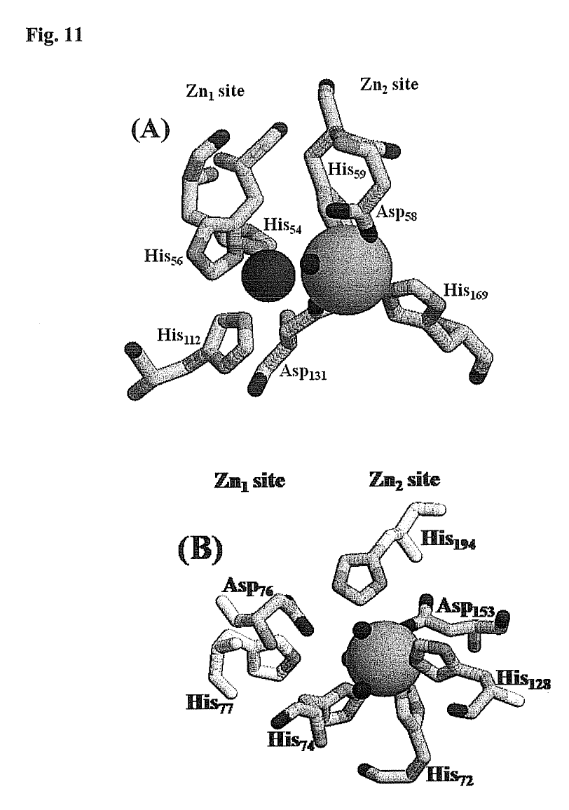

Results presented in this work represent the first detailed characterization of an ETHE1 protein from any organism. ETHE1 is most similar to the GLX2-family of proteins, which are members of the metallo-β-lactamase superfamily. This superfamily consists of proteins that catalyze a wide range of reactions but share the common metal binding motif H-x-[EH]-x-D-[CRSH]-X50-70-[CSD]-X (SEQ ID NO: 78), which is part of the common β-lactamase fold. This motif typically consists of two metal ions that are essential for the activity of the majority of the enzymes. In most β-lactamase fold containing proteins, the coordination of the first metal (Zn₁ site) is tetrahedral and consists of three histidines and a bridging hydroxide, while the second metal binding site (Zn₂ site) is trigonal pyramidal. The site 2 metal binding ligands are more variable but always contain a histidine and aspartic acid (FIG. 11A). Because of the similarity of AtETHE1 to the GLX2 family of enzymes, it was predicted that AtETHE1 would also bind two equivalents of metal. Metal analyses of AtETHE1 in Example 2 were based on a calculated extinction coefficient of 10,240 M⁻¹ cm⁻¹. Metal analyses using this extinction coefficient indicated that iron-bound AtETHE1 contained two equivalents of iron; however, the AtETHE1 crystal structure reported only a 0.5 iron metal occupancy. It was originally thought that this discrepancy resulted from the loss of metal during the crystallization processes. However, results presented here suggest that the extinction coefficient used for these experiments is inaccurate. When using amino acid analyses, an extinction coefficient of 7,240 M⁻¹cm⁻¹ was obtained, which leads to a metal:protein stoichiometry of 1.2±0.2. This number is consistent with the AtETHE1 crystal structure shown in Example 2.

Consistent with the crystal structure of AtETHE1, our results suggest that AtETHE1 contains a single Fe(II) that is coordinated by two histidines (FIG. 9). GLX2 enzymes enriched in iron typically contain an antiferromagnically-coupled Fe(II)-Fe(III) center in their dinuclear metal binding site. Results from our EPR studies argue against the presence of an Fe(II)-Fe(III) site in AtETHE1. Two distinct EPR signals were observed for AtETHE1, both due to magnetically-isolated Fe(III) and both accounting for only a very small proportion (1-5%) of the total iron. Even extensive exposure to air was unsuccessful in increasing the Fe(III) signal. The very low intensities of the Fe(III) signals can either be due to most of the iron being in the Fe(II) state or most of the iron residing in an anti-ferromagnetically-coupled S′=0 dinuclear site. The lack of signals due to an Fe(III)-Fe(II) center and the crystallographic identification of a monometallated active site both argue against a predominant Fe(III)-Fe(III) S′=0 species and support Fe(II) as the predominant oxidation state of the metal in AtETHE1.

Even though AtETHE1 contains all of the highly-conserved metal binding ligands of the metallo-β-lactamase family of proteins, AtETHE1 apparently does not bind two equivalents of metal. In agreement with this result, the crystal structure identified changes in the tertiary structure of the metal binding domain that do not allow for the coordination of a second metal atom (FIG. 11B). A single-turn helix in AtETHE1 containing His112 is pulled away from the active site, displacing His112 away from the metal atom in AtETHE1 relative to GLX2-5. This unwinding of the helix also displaces the side chains of Asp115 and His116, which have been shown to coordinate the second metal in GLX2-5 (FIG. 11). Therefore, subtle changes in protein conformation have displaced several of the metal binding ligands, ultimately limiting the ability of AtETHE1 to bind two metal ions.

There are other examples of metallo-β-lactamases that only bind one metal. CphA from Aeromonas hydrophila and GOB-1 from Elizabethkinga meningosptica both only bind a single Zn(II) atom in the Zn₂ site (Asp, Cys, His). However, in these enzymes, the Zn₁ site is altered either by the replacement of a histidine with an asparagine in CphA or a histidine with glutamine in GOB-1. To our knowledge AtETHE1 is the first example of a β-lactamase family protein that contains all of the conserved metal-binding ligands, yet only binds one metal ion.

In addition, it has been shown that the presence of soft or hard ligands in the metal binding site can affect the specificity of the metal binding. All of the metallo-β-lactamases exclusively bind Zn(II). The incorporation of an aspartic acid and an additional histidine in the Zn₂ site likely allows for the variable binding of Fe, Zn, and Mn seen in the GLX2 enzymes (FIG. 11A). Likewise, the replacement of two soft ligands by an aspartic acid and a glutamate as observed in ROO allows the formation of a di-iron center. Interestingly, in the AtETHE1 crystal structure, the Fe ion is bound in the Zn₁ site that has been modified by the removal of a histidine and the shifting the GLX2 bridging aspartic acid to specifically coordinate the iron (FIG. 11B and Example 2). The Fe(II) ion is further coordinated by three water molecules resulting in an octahedrally-bound metal, unlike the tetrahedral coordination of metal normally seen in the Zn₁ site of metallo-β-lactamases.

Therefore, AtETHE1 proteins appear to represent a new class in the metallo-β-lactamase fold family of proteins. Although structurally similar to the GLX2 enzymes, AtETHE1 appears to have evolved to bind a single iron atom in an octahedral configuration. Metal analyses and spectroscopic data suggest that unlike GLX2 enzymes AtETHE1 tightly binds to a single Fe(II) atom in a modified Zn₁ site of the metallo-β-lactamase metal binding motif. Finally we show that AtETHE1 is homodimeric in solution and exhibits low levels of esterase activity, suggesting that AtETHE1 might hydrolyze a short-chain ester.

### Example 4

### Role of AtETHE1 in Plant Response to Abiotic Stresses and its Distribution

This Example describes observed enhanced expression of AtETHE1 in response to abiotic stresses. This provides evidence that AtETHE1 is involved in a plant stress response. This Example shows the ubiquitously expression of AtETHE1 throughout the plant suggesting a possible role as a housekeeping gene.

Materials and Methods

Plant Material:

Stress studies were performed on Wild-type Arabidopsis Columbia grown hydroponically. Seeds were germinated on Rockwool (GrodanHP, Agro Dynamics Inc., East Brunswick, N.J., USA) suspended above Hoagland's solution and grown in a growth chamber maintained at 23° C. and 16 hour light, 8 hour dark cycles. For the plants stressed with abscisic acid (ABA), seedlings were grown hydroponically for 7 days and then stressed for 3 hours with 10 uM ABA. Plants were grown hydroponically for 18 days for stresses with NaCl and Mannitol. Plants were either stressed with 150 mM NaCl for 12 hours or stressed for 24 hours with 300 mM Mannitol. Plant material, roots only for NaCl stress, was immediately ground in liquid N₂ and stored in the −80° C. until needed for RNA isolation.

Molecular Analysis:

AtETHE1 RNA levels were determined using RT-PCR on total RNA isolated from either whole plants (ABA, Mannitol treated) or from the roots (NaCl treated). AtETHE1 cDNA was synthesized with equal amounts of RNA (1 μg) using a Thermoscript RT-PCR kit and mRNA levels were measured by 25 cycles of PCR using the primer pair 2-3-5′ (5′-TGGACAAGACTGTGGATAGAGA) (SEQ ID NO: 21) and 2-3-2 at using an annealing temperature of 56° C. for 1 minute. ACT8 was used as a control.

Role of AtETHE1 in Plant Response to Abiotic Stresses

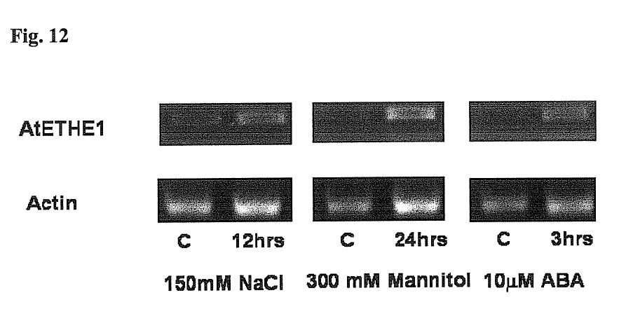

It has been shown in various microarray data accessed from the TAIR databank (www.Arabidopsis.org) that AtETHE1 has enhanced expression for abiotic stresses, including: abscisic acid (ABA), NaCl, and mannitol. To verify these results, wild-type Arabidopsis Columbia plants were grown hydroponically to allow for controlled infiltration of each stress and an expression analysis was performed using reverse-transcription polymerase chain reaction (RT-PCR). Enhanced expression was observed compared to the control of all three abiotic stresses (FIG. 12). Treatment with ABA and NaCl resulted in 1.8 fold and 2.1 fold increases respectively. Treatment with mannitol resulted in the largest fold increase of 2.6. These results verify the published microarray data from the TAIR databank. All three abiotic stresses are involved in several types of water limiting stress including salinity and drought. These results therefore provide implications for AtETHE1's role in drought and osmotic stresses.

Distribution of AtETHE1 in Arabidopsis thaliana

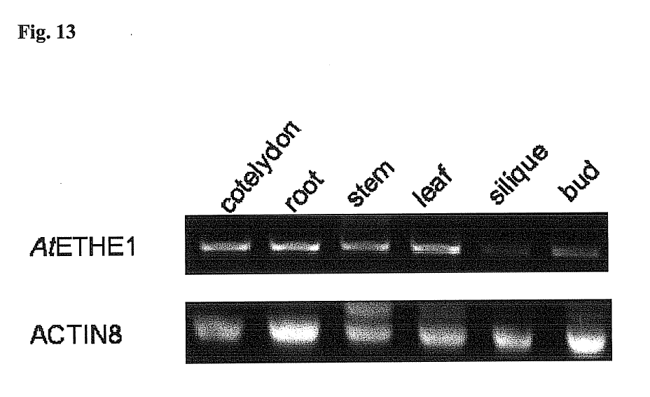

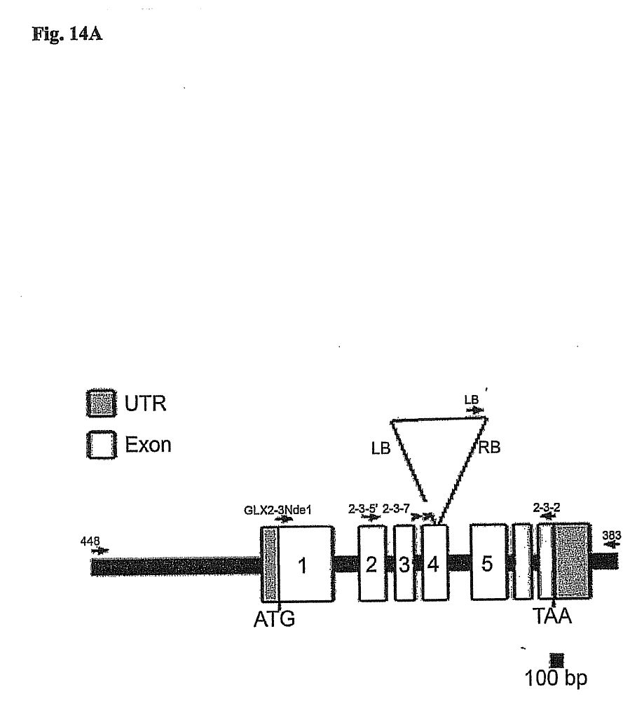

To provide insight into the possible role of AtETHE1, an expression analysis was performed on several different tissues of Arabidopsis: cotyledon, root, stem leaf, silique, and bud (FIG. 13). Results show that AtETHE1 is ubiquitously expressed throughout the plant with lower levels of expression seen in the silique and bud. This data provides implications that AtETHE1 is a housekeeping gene.

For distribution analysis, total RNA was isolated from 7 day old Arabidopsis seedlings for cotyledon and root tissues, and fully mature plants for stem, leaf, silique, and bud tissues. AtETHE1 cDNA was synthesized with equal amounts of RNA (1 μg) a Thermoscript RT-PCR kit and mRNA levels were measured by 25 cycles of PCR using the primer pair 2-3-5′ (5′-TGGACAAGACTGTGGATAGAGA) (SEQ ID NO: 21) and 2-3-2 using an annealing temperature of 56° C. for 1 minute. ACT8 was used as a control.

### Example 5

### ETHE1 is Essential for Embryo and Endosperm Development

To investigate the role(s) of ETHE1, we characterized the Arabidopsis thaliana ETHE1 homolog (AtETHE1) and investigated the effect of an AtETHE1 loss-of function mutation. Seeds homozygous for a T-DNA insertion in AtETHE1 exhibit an early arrest in endosperm development that is followed by embryo arrest beginning at heart stage resulting in seed lethality. Seeds lacking AtETHE1 exhibit endosperm defects as early as the 4 cell zygote stage followed by an arrest in embryo development by early heart stage. Strong AtETHE1 labeling was observed in the peripheral and chalazal endosperm of wild-type seeds prior to cellularization. Taken together, these results demonstrate that AtETHE1 is essential for endosperm development and ultimately embryo development in plants.

Experimental Procedures

Plant material. The Arabidopsis thaliana AtETHE1 loss of function mutant was obtained from the DuPont p2800 T-DNA tagged seed pool. Seeds were vernalized at 4° C. for 48 hours prior to being placed in a growth chamber for growth on commercial potting soil at 23° C. with a 16:8 hour light/dark cycle. Wild-type Wassilewskja (WS) was used as the control in the development and immunolocalization studies.

Stress studies were performed on wild-type Arabidopsis Columbia plants grown hydroponically. Seeds were germinated on Rockwool (GrodanHP, Agro Dynamics Inc., East Brunswick, N.J., USA) suspended above Hoagland's solution (Cowgilland & Milazzo, (1989) The Culturing and Testing of Two Species of Duckweed. In W J Adams, G A Chapman, eds, Aquatic Toxicology and Hazard Assessment Vol 12. American Society for Testing and Materials, Philadelphia, Pa., pp 379-391) and grown in a growth chamber maintained at 23° C. with 16 hour light, 8 hour dark cycles. Plants stressed with abscisic acid (ABA), were grown hydroponically for 7 days and then stressed for 3 hours with 10 μM ABA. Plants were grown hydroponically for 18 days prior to treatment with either 150 mM NaCl or 300 mM Mannitol for 24 hours.

Phylogenetic Analysis of β-Lactamase Proteins.

A phylogenetic tree was derived from multiple alignments of β-lactamase-fold containing proteins using Clustal W version 1.82. A neighbor joining phylogenetic analysis was conducted with MEGA version 3.1 using the Poisson correction amino acid substitution model and the complete deletion gaps option (Kumar, et al., (2004) MEGA3: Integrated Software for Molecular Evolutionary Genetics Analysis and Sequence Alignment. Brief Bioinform 5: 150-163). Bootstrap values from 500 replicates were calculated and are indicated at branch points on the neighbor-joining tree. The phylogenetic analysis included H. sapiens ETHE1 (NP_{—}055112), M. musculus ETHE1 (NP_{—}075643), Xenopus laevis ETHE1 (NP_{—}001079404), Xenopus tropicalis ETHE1 (NP_{—}001005706), Arabidopsis ETHE1 (NP_{—}974018), O. sativa Os01g066200 (NP_{—}001043807), Burkholderia phytofirmans (ZP_{—}01508561), Stigmatella auranticaca ETHE1 (ZP_{—}01459266), Myxococcus xanthus (YP_{—}633997), Arabidopsis GLX2-1 (NP_{—}973679), Arabidopsis GLX2-2 (NP_{—}187696), Arabidopsis GLX2-4 (NP_{—}849599), Arabidopsis GLX2-5 (NP_{—}850166), H. sapiens GLX2 (CAA62483), M. musculus GLX2 (NP_{—}077246), O. sativa GLX2 (AAL14249), Brassica juncea GLX2 (AA026580), S. cerevesia GLO2 (CAA71335), S. cerevesia GLO4 (CAA99230), O. sativa Os03g0332400 (BAF11930), O. sativa OsJ_{—}010290 (EAZ26807), and Stentrophomonas maltophilia L1 β-lactamase (CAB63489).

Molecular Analysis of ETHE1.

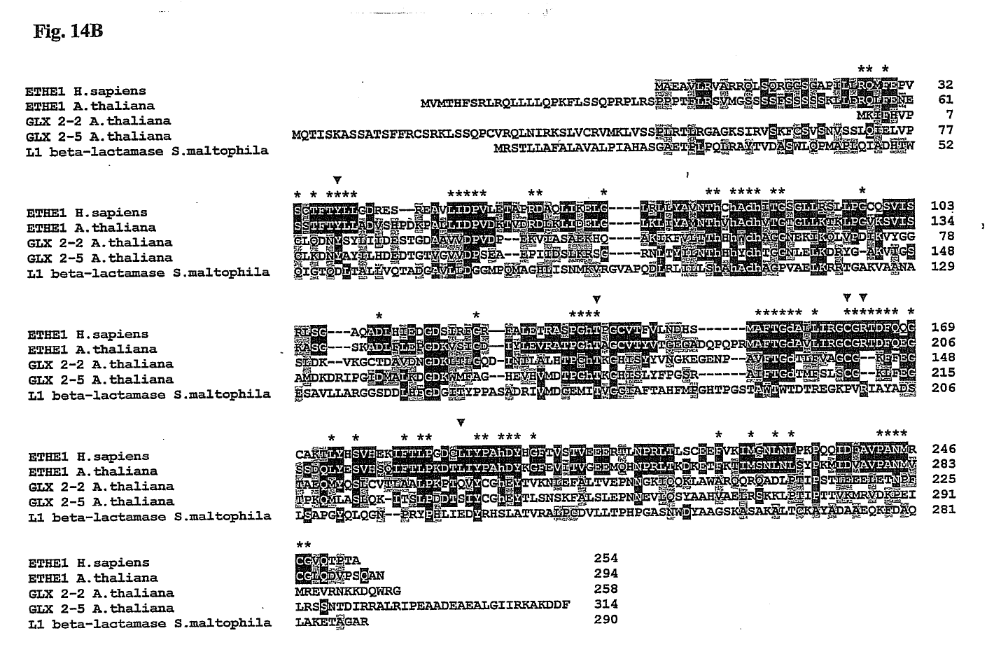

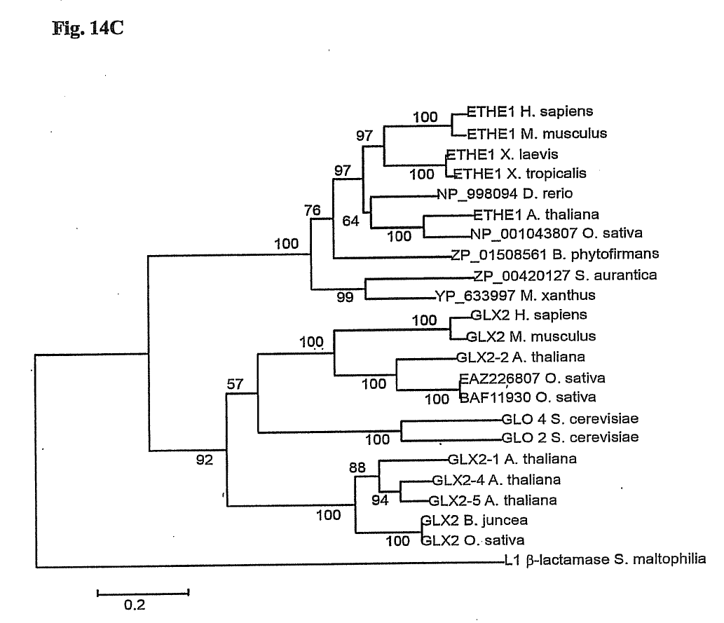

AtETHE1 cDNA was reverse transcribed from Arabidopsis total bud RNA and PCR amplified using primers GLX2-3NdeI (FIG. 14) and a 3′ poly (A) anchor primer, generating a NdeI site at the predicted amino terminus of AtETHE1 and a XhoI site at the end of the cDNA, respectively. The amplified DNA was cloned into the expression vector pET15b (Novagen), sequenced and introduced into E. coli BL21-RIL cells for protein over-expression.

Cell cultures of E. coli BL21-RIL cells containing the AtETHE1 plasmid were grown, induced and AtETHE1 purified as described previously (Crowder, et al., (1997) FEBS Lett 418: 351-354). Protein purity was assessed by SDS-PAGE gels and quantified at Abs₂₈₀ using a molar extinction coefficient of 10,240 M⁻¹cm⁻¹. This value was determined using the method of Gill and von Hippel (Gill & von Hippel, (1989) Anal Biochem 182: 319-326). The enzymatic activity of AtETHE1 with S-D-lactoylglutathione (Sigma) was measured as previously described (Marasinghe, et al., (2005) J. Biol. Chem. 280: 40668-40675). For antibody production purified AtETHE1 (280 mg) was resuspended in 1 ml PBS and mixed with either Freund's complete or incomplete adjuvant (Sigma) and used to raise polyclonal antibody in rabbits using standard procedures.

AtETHE1 RNA levels were determined by RT-PCR on total RNA (1 μg) isolated from cotyledon, root, stem, leaf, silique, and bud, and seedlings subjected to abiotic stresses. AtETHE1 cDNA was synthesized using a Thermoscript RT-PCR kit, followed by 25 cycles of PCR using the primer pair 2-3-5′ and 2-3-2. ACT8 was used as a control (An, et al., (1996) Plant J 10: 107-121).

A T-DNA insertion in AtETHE1 was identified by performing PCR on pooled genomic DNA isolated from a population of Arabidopsis T-DNA insertion lines as previously described (Krysan, et al., (1996) Proc Natl Acad Sci USA 93: 8145-8150; Todd, et al., (1999) Plant J 17: 119-130). T1 seeds from pool p2800 containing the AtETHE1 T-DNA line were obtained from the ABRC. Genomic DNA was isolated and screened by PCR to identify plants containing the AtETHE1 T-DNA insertion. Genotyping was performed by PCR using a combination of T-DNA insertion and wild type AtETHE1 primer pairs. Amplification products were verified through DNA sequencing.

Complementation studies were conducted in pCAMBIA-1390 containing a 3400 bp BamH1 and EcoR1 genomic DNA fragment amplified from the T3F20 BAC clone with primers 448 and 383. The construct was transformed into GV3101/PMP90 Agrobacterium cells and transferred into a segregating population of heterozygous ETHE1/ethe1 plants (Chung, et al., (2000) Transgenic Res. 9: 471-476. Transformants were selected on hygromycin (25 mg/L) and confirmed by PCR screening. Genotyping of hygromycin-resistant plants was performed by PCR using primers specific to the T-DNA insertion (primer pair LB2/2-3-2), and the wild-type AtETHE1 locus (primer pair 609 and 618 (AGTAAGTTGTGTTGTGTCACAC)) (SEQ ID NO: 22). The ability of the complementation construct to rescue the ethe1 phenotype was determined through the analysis of 40 mature siliques of ethe1 plants.

Microscopy.

Mature siliques (84) from ETHE1/ethe1 plants and wild-type plants were treated with a 4% (w/v) sodium hydroxide solution for 16 hours at room temperature and the numbers of aborted and healthy seeds were determined by examination under a dissecting microscope.

Embryo and endosperm development were analyzed in siliques from 40 ETHE1/ethe1 plants at various stages of development using Laser Scanning Confocal Microscopy (LSCM) essentially as described (Braselton, et al., (1996) Biotech Histochem 71: 84-87). The samples were cleared and individual seeds were dissected in methyl salicylate, and viewed using an Olympus IX-81 fluorescence deconvolution microscope system. Data were analyzed with Image Pro Plus (Media Cybernetics) and organized with Photoshop.

SEM was performed on staged flowers/siliques fixed overnight at room temperature in 2.8% (v/v) glutaraldehyde in 0.1 M HEPES buffer (pH 7.2) and 0.02% (v/v) Triton X-100) and post-fixed in 1% (w/v) aqueous OsO₄ overnight. Individual gynoecia were dissected open in drops of buffer to expose the ovules and placed into a critical point drier specimen basket submerged in buffer. Samples were dehydrated through a graded ethanol series (10% increments, one hour each) with 3 changes of 100% ethanol and critical point dried with a Balzers CPD 020 using CO₂ as the transitional fluid. Dried specimens were mounted, sputter-coated with gold and examined with a Hitachi S-570 SEM operating at 15 kV. Digital images were captured using an Oxford Link ISIS microanalysis system.

Immunolocalization studies to examine the distribution of AtETHE1 were conducted on flowers and siliques 0.4 cm-0.8 cm in length. Slides were prepared as described (Hong, et al., (1996) Plant Mol Biol 30: 1259-1275). Blocks were sectioned at 12 microns on a microtome and adhered to poly-L-lysine coated slides. Immunochemistry was performed essentially as described (Shiba, et al., (2001) Plant Physiol. 125: 2095-2103) using anti-ETHE1 primary antibody (1:1000) and the alkaline-phosphatase goat-anti-rabbit secondary antibody (1:1000). Samples were observed and analyzed as described above.

Results

Molecular analysis of ETHE1. The Arabidopsis gene At1g53580 (FIG. 14A) had previously been predicted to encode a glyoxalase II isozyme. However, after further analysis we show here that it represents Arabidopsis ETHEL Analysis of a cDNA (NM_{—}202289) for At1g53580 revealed that it has the potential to encode a 294 amino acid protein that belongs to the β-lactamase family of proteins. A pairwise protein alignment between At1g53580 and the Arabidopsis cytoplasmic glyoxalase II (GLX2) isozyme (AtGLX2-2) revealed only 13% identity, while a pairwise alignment between At1g53580 and human ETHE1 revealed 54% identity, indicating that At1g53580 is likely not a GLX2 enzyme, but rather Arabidopsis ETHE1 (FIG. 14B).

GLX2 and ETHE1 along with A-type flavoproteins and zinc phosphodiesterases belong to the metallo-β-lactamase family of proteins, which are defined by a common αβ/βα fold and a conserved metal binding motif, T-H-X-H-X-D (SEQ ID NO: 23). While ETHE1 enzymes are most similar to the GLX2 family of proteins, there are several biological and structurally distinguishing features of AtETHE1 that set it apart from the GLX2 family. Sequence comparisons led to the prediction that AtETHE1 does not utilize SLG as a substrate. In order to test this prediction, Arabidopsis ETHE1 was over-expressed, purified, and its activity with SLG was tested. As predicted, AtETHE1 exhibited no enzymatic activity towards SLG, confirming that it is not a GLX2 enzyme. In addition, GLX2 enzymes function as monomers; however, gel filtration studies on AtETHE1 showed that it exists as a dimer. Taken together, these results confirm that At1g53580 is not a GLX2 enzyme, but rather is the Arabidopsis ETHE1 homolog.

A Blast-P search revealed that ETHE1-like proteins are present in most organisms including animals, plants, fungi, and bacteria. Interestingly, an ETHE1-like protein does not appear to be present in yeast. A neighbor-joining tree was generated based on a Clustal W alignment of select metallo-β-lactamase proteins including ETHE1-like and GLX2 proteins. Analysis of the phylogenic tree, rooted in the common ancestor β-lactamase, demonstrates that β-lactamases diverged through ancient duplication events into two separate lineages: ETHE-1 like and GLX2 proteins (FIG. 14C). As expected, AtETHE1 grouped together with human ETHE1 and predicted ETHE1 proteins from mouse, frog, fish, rice, and bacteria. Likewise, the Arabidopsis GLX2 enzymes grouped together with GLX2 proteins from human, mouse, rice, broccoli, and yeast (FIG. 14C). In addition to the conserved metal-binding residues, all ETHE1 proteins share a number of conserved residues, including R163, C161, Y38, L185, and T136 (FIG. 14B). Mutations in several of these residues have been associated with EE in humans, suggesting that they are required for catalytic activity. We have therefore renamed GLX2-3 as AtETHE1.

AtETHE1 is Required for Early Seed Development

In order to gain insights into the role of ETHE1 in general and plants in particular an Arabidopsis line containing a T-DNA insertion in the gene was isolated and characterized. PCR screening of a population of T-DNA insertion mutants resulted in the identification of a line that contained a T-DNA insertion in exon four of AtETHE1 (FIG. 14 a). In a segregation analysis on the progeny of ETHE1/ethe1 plants, 312 (32%) were homozygous for the wild-type locus and 673 (68%) were heterozygous for the AtETHE1 T-DNA insertion. No plants homozygous for the T-DNA insertion were identified. The 1:2:0 segregation ratio [χ²=1.33<χ_{0.05(1)}²=3.841] suggested that ETHE1 is an essential gene and that the mutation may result in embryo lethality.

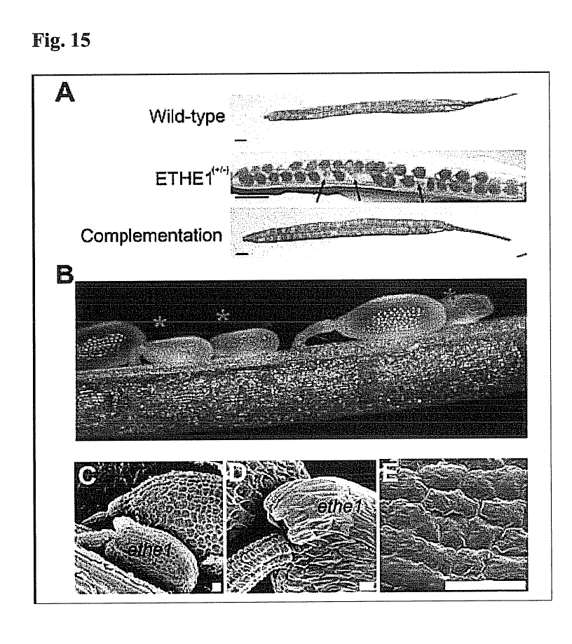

Seed formation was then investigated in mature siliques to determine if in fact the ETHE1 T-DNA insertion is associated with alterations in seed development. A total of 107 siliques from 51 self-pollinated AtETHE1/ethe1 plants were examined and compared to those of wild-type plants. Approximately 4% (27/638) of the seeds contained in wild-type siliques appeared aborted, defined as small masses (FIG. 15A). In contrast, siliques of AtETHE1/ethe1 plants contained 24.2% (805/3353) aborted seeds [χ²=1.81<χ_{0.05(1)}²=3.841], which appeared as shrunken, shriveled masses, suggesting that the mutation does in fact affect seed development.

Analysis of seed development in immature siliques of AtETHE1/ethe1 plants revealed several phenotypic differences between mutant and wild-type seeds. The most obvious alteration is that Atethe1 seeds are dramatically smaller in size than their wild-type counterparts (FIG. 15B). Differences in seed size were first detected in siliques 7-8 mm in length. The reduced size of Atethe1 seeds relatively early in development suggested that the mutation may affect early endosperm development, which is coupled to cell elongation and development of the seed coat. Consistent with this hypothesis, analysis of developing seeds using Scanning Electron Microscopy (SEM) revealed no differences between seeds in the siliques of AtETHE1/ethe1 plants at early stages in seed development (data not shown). However, at later stages, cells of the outer integument in Atethe1 seeds appeared smoother and less well defined than those of wild-type and heterozygous seeds in the same silique (FIGS. 15C and 2D). This is similar to the appearance of the integument during earlier stages of wild-type seed development (FIG. 15E), suggesting that seed coat development arrests relatively early in the development of Atethe1 seeds.

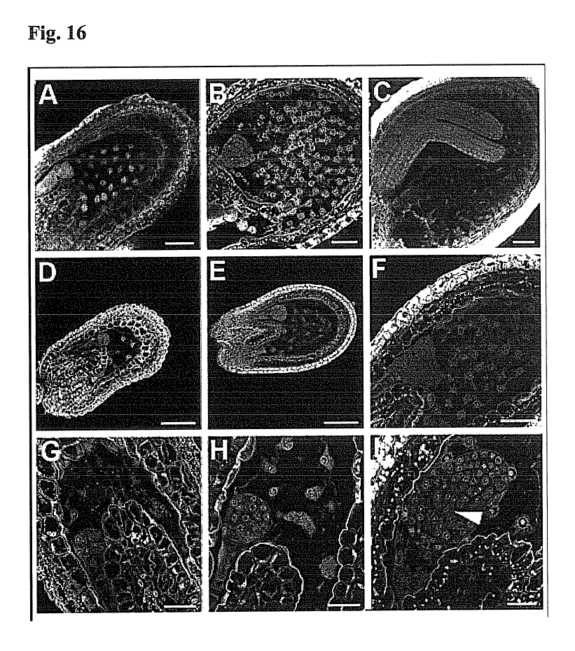

Embryo and endosperm development was then investigated in siliques of AtETHE1/ethe1 plants using Laser Scanning Confocal Microscopy to more specifically determine the nature of the defect. Although differences in seed size were already apparent, no obvious alterations in embryo development were observed in the seeds of AtETHE1/ethe1 siliques early in development, including at the zygote and 4-8 cell globular stages (compare FIGS. 16A and D). The first clear developmental difference in embryo development was observed when wild-type embryos were at early heart stage. When most seeds were at heart stage, 23% (129 of 560) of the seeds in AtETHE1/ethe1 siliques, all of which were small in size, remained at the 32 cell globular stage (compare FIGS. 16B and E). By the time most of the seeds had progressed to late torpedo and cotyledon stages, Atethe1 seeds in the same silique had only developed to early heart stage (compare FIGS. 16C and F). With the exception of the delayed/arrested development, no dramatic cellular abnormalities were detected in Atethe1 embryos during early and late globular stages (FIG. 16G-H). However, at heart stage some Atethe1 embryos contained elongated cells (FIG. 161).

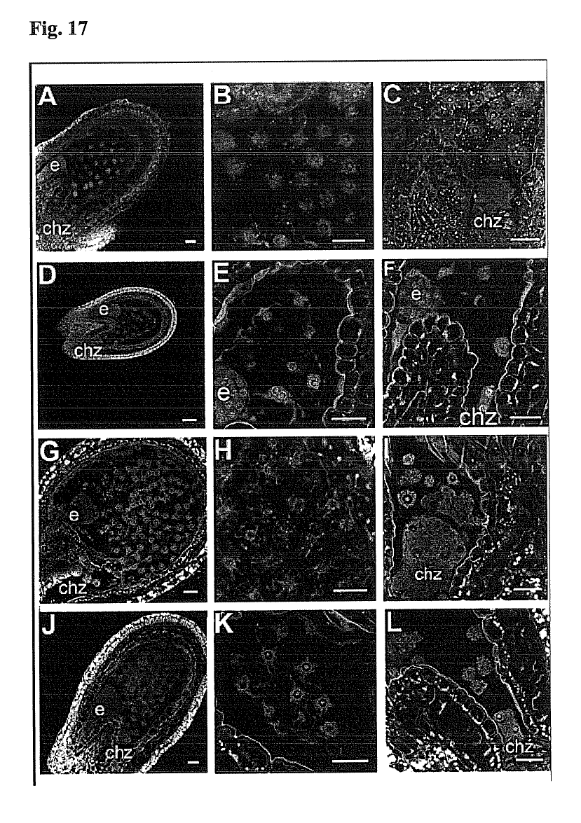

As discussed above, the overall size of Atethe1 seeds at the globular stage of development is much smaller than wild-type seeds at the same developmental stage (compare FIG. 17A-D,B-E). Several studies have shown that the endosperm plays a direct role in early seed size, suggesting that the ethe1 mutation may cause defects in endosperm development. A detailed analysis of endosperm development revealed that this is in fact the case. Specifically, Atethe1 seeds have fewer endosperm nuclei than wild-type seeds at most developmental stages. These differences are most pronounced in the PEN region (compare FIGS. 17B and H, and 17E and K), which accounts for the majority of the seed's size. In a developing wild-type seed at the 8-cell octant stage, endosperm nuclei in the PEN region have undergone 7 rounds of division resulting in approximately 100 free endosperm nuclei (FIG. 17A-B). Cellularization of the endosperm begins shortly after this stage progressing in a wave from the micropylar region through the CZE by the end of heart stage. This, however, is not the case in ethe1 seeds. In contrast to the ca. 100 free endosperm nuclei in the PEN region of wild-type seeds, only 10-15 endosperm nuclei were observed in globular Atethe1 seeds (FIG. 17D-E). By the time Atethe1 seeds approached heart stage, only 40-50 endosperm nuclei were observed in comparison to the 200 nuclei seen in the wild-type PEN region (compare FIG. 17G-H and FIG. 17J-K). In addition, unlike wild-type seeds, the endosperm of Atethe1 seeds did not undergo cellularization (compare FIGS. 17H and K).

Alterations in the CZE were noted in the mutant beginning at the 4-cell zygote stage. The CZE in Atethe1 seeds (FIGS. 17F and L) appeared underdeveloped, containing very little cytoplasm surrounding the chalazal cyst and few, if any, chalazal nodules compared to wild-type seeds at the same developmental stage (FIGS. 17C and I). Therefore, defects are observed in the endosperm as early as the 4-cell zygote stage. This suggests that a primary effect of the ethe1 mutation is disruption of endosperm development, which in turn may slow and ultimately cause the arrest of embryo development.

The tight genetic linkage between the AtETHE1 T-DNA insertion and the observed embryo lethality indicated that AtETHE1 is an essential gene that is required for early embryo and endosperm development. A complementation study was performed to verify this conclusion. A 3.4 Kbp genomic DNA fragment containing AtETHE1 was transformed into a segregating population of AtETHE1/ethe1 plants. Three independent lines that were homozygous for the Atethe1 T-DNA mutation and contained the AtETHE1 complementation construct were identified. To further verify that complete complementation had occurred, individual siliques from Atethe1 plants, which contained the complementation construct, were analyzed for the presence of abnormal/aborted seeds. A total of 59 (3.7%) out of 1341 seeds examined in siliques from the complementation lines were aborted, which is similar to the number of aborted seed typically observed in wild-type siliques (FIG. 15A). These results confirm that the Atethe1 mutation is responsible for the seed defects we observed.

AtETHE1 Expression and Localization

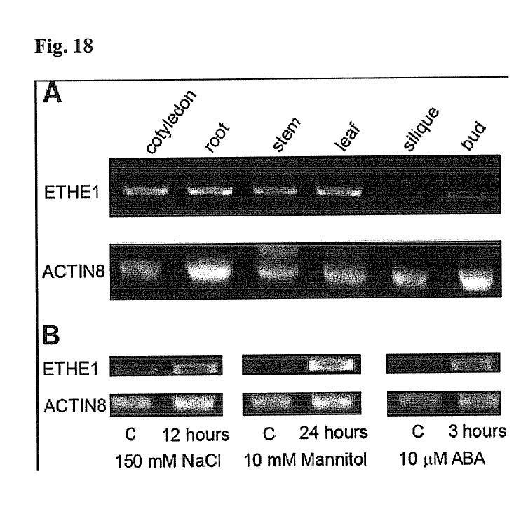

In order to determine if AtETHE1 plays a role throughout plant development we investigated its expression patterns. AtETHE1 is present on both the Affymetrix AG: 8 k array and the ATH1: 22 k array Genechip®. Therefore there is a considerable amount of information concerning AtETHE1 expression patterns in the public databanks (Zimmermann P, et al. (2005) Trends in Plant Science 10:407-409). Analysis of the available data indicated that AtETHE1 RNA is present in every tissue examined with the lowest levels detected in reproductive tissues. In order to confirm these results, the distribution of AtETHE1 transcripts in various tissues was determined using RT-PCR. The ACTIN8 (ACTS) gene was used to standardize the reactions due to its relatively constant expression. AtETHE1 transcript levels were detected in all tissues examined. Consistent with the microarray data, lower levels were seen in siliques and buds (FIG. 18A). Further analysis of the microarray data revealed that AtETHE1 transcript levels are elevated during senescence, osmotic stresses (e.g. mannitol and NaCl), and biotic stresses (e.g. infection with B. cinerea and P. syringae). Significant changes (greater than 2 log) were not observed under other conditions. To verify these results, wild-type Arabidopsis Columbia plants were grown hydroponically, subjected to several different stresses and AtETHE1 RNA levels were determined. Three different abiotic stresses (mannitol, NaCl, and abscisic acid) resulted in elevated AtETHE1 transcript levels (FIG. 18B). Treatment with ABA and NaCl resulted in approximately two fold increases in AtETHE1 transcript levels while treatment with mannitol resulted in an increase of approximately 2.5 fold. Therefore, AtETHE1 transcripts are present throughout the plant and their levels increase during stress.

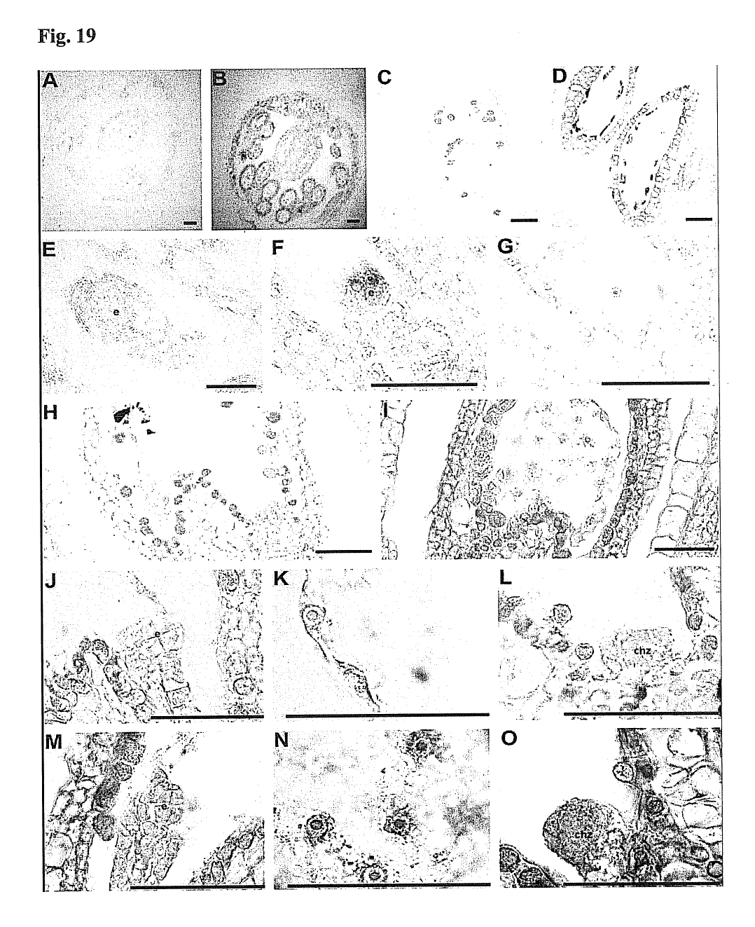

We next investigated the distribution of AtETHE1 during flower and seed development in order to investigate why the Atethe1 mutation appears to have a more pronounced effect on endosperm development. Immunolocalization studies on buds, flowers, and developing siliques revealed AtETHE1 signals above background levels in all tissues examined (FIG. 19). Consistent with the results from our RT-PCR experiments, reproductive organs in general do not contain high levels of AtETHE1. However, strong AtETHE1 signal is present in the tapetal cells, the nutrient cells of the anther (FIG. 19A-D).

In early stages of seed growth, including from the zygote to the globular embryo stages, AtETHE1 signal is present in the developing embryo and throughout the endosperm (FIG. 19E-I). Closer examination revealed that early in development the strongest AtETHE1 signal is observed in the nuclei of the embryo proper and the PEN region of young, zygote-staged, seeds (FIGS. 19F and G). As development progresses into the globular stage, AtETHE1 signal becomes more pronounced and is the strongest in the PEN and CZE tissues (FIG. 19M-O). At this stage in embryo development, the MCE has begun to cellularize, while the PEN and CZE are still rapidly dividing. The strongest AtETHE1 signal is observed in PEN endosperm nuclei and at the base of the chalazal cyst, consistent with the theory that AtETHE1 plays an important role in early endosperm development (FIG. 19N-0).

Discussion

AtETHE1 is Essential for Early Endosperm Development

CLSM studies of seed development in AtETHE1/Atethe1 plants first identified developmental delays in both the PEN and CZE beginning soon after fertilization with most endosperm nuclei arresting after only a few divisions (FIG. 17E-F, 17K-L). Typically fewer than 50 endosperm nuclei were observed in the PEN region of Atethe1 seeds along with only a few chalazal nodes (FIGS. 17F and L). In addition, cellularization of the endosperm never occurred in Atethe1 seeds. Delays in embryo development were observed beginning at globular stage with embryo arrest occurring at heart stage (FIG. 16D-F). With the exception of the developmental delay and the presence of small numbers of elongated cells in some heart-stage embryos, alterations were not observed in Atethe1 embryos (FIG. 16G-I). The observation that the earliest and most dramatic effects are on the endosperm suggest that the mutation primarily affects endosperm development. Consistent with this is our observation that while AtETHE1 is present in both embryo and endosperm tissue during very early stages of seed development (FIG. 19G-H), the strongest AtETHE1 signals are observed in endosperm nuclei of the PEN region and the chalazal cyst as development progresses towards the globular stage (FIG. 19M-O). Therefore, we believe that the primary defect of Atethe1 seeds is in the endosperm and that defects in early endosperm development ultimately result in embryo arrest by heart stage. However, it is possible that AtETHE1 is also critical for embryo development and embryo arrest results from the lack of AtETHE1 activity and not from the underdeveloped endosperm.

Several mutants have been isolated showing defects in late endosperm development, including the absence of endosperm cellularization. The majority of these mutations such as the pilz, titan, knolle and hinkel mutants are in genes required for nuclear or cellular division and affect both the embryo and endosperm. Several other mutants, including spatzle, fis1, and fis2 have been identified where no apparent cytokinesis defects are observed in the developing embryo, however the endosperm fails to cellularize. Interestingly, even though endosperm cellularization is blocked in these mutants, the resulting seed is comparable in size to wild-type and is able to develop into a fully functional plant, suggesting that late endosperm development is not required for embryo viability. Therefore, the absence of endosperm cellularization is likely not the cause of the embryo arrest in Atethe1 seeds.

Analysis of the haiku2 mutant showed that defects in endosperm development can affect integument elongation and seed size. The reduced size and delayed/restricted development of the integument in Atethe1 seeds is consistent with the theory that early endosperm development plays an important role in seed size and integument development. The endosperm of Atethe1 seeds arrests at a much earlier stage of development than do haiku2 seeds, which exhibit precocious endosperm cellularization, reduced proliferation of the endosperm and a reduction of embryo growth at the torpedo stage. Interestingly, haiku2 seeds while smaller than wild-type are still viable. Our data together with these results suggest that a critical number of syncytial mitoses are required to support the development of a viable embryo.

Consistent with this hypothesis are the results of a study in which endosperm-specific expression of diphtheria toxin shortly after the second round of nuclear divisions resulted in small seeds in which the embryo arrested at heart stage. Prior to arrest, the embryos in the KS221>>DTA seeds exhibited a number of abnormalities. In particular, preglobular embryos displayed swollen protodermal cells and/or altered division planes; however, they did not show signs of cell death. Defects in Atethe1 seeds are not as dramatic as those observed in the KS221>>DTA seeds. In particular, the endosperm in Atethe1 seeds undergoes several rounds of nuclear division, and while embryos arrest at heart stage, they do not exhibit distorted division patterns. Taken together these results suggest that the endosperm may play several roles in embryo development. Early embryo development is closely tied with endosperm development with the first several syncytial divisions being critical to establish proper division patterns in the embryo. Later, a threshold level of endosperm nuclei appears to be necessary to support the growth and development of the embryo beyond heart stage. Our results are consistent with the predicted role of the CZE in the uptake, processing, and transfer of metabolites to the endosperm and suggest that the chalazal cyst is necessary for embryo development beyond heart stage. Once the embryo has developed beyond heart stage the endosperm appears to be dispensable.

In summary, we have shown that AtETHE1 is essential for early aspects of endosperm development with defects occurring soon after fertilization. To our knowledge, Atethe1 is one of the earliest characterized mutants that affect endosperm development. Embryo development subsequently arrests at heart stage, supporting the link between early embryo and endosperm development.

### Example 6

### Over-Expression of Arabidopsis ETHE1 Results in Enhanced Plan Growth Properties

We demonstrate here that Arabidopsis AtETHE1 is normally localized in the mitochondrion. Using MALDI-TOF peptide mapping, we identify the mature N-terminus of the protein. Because ETHE1 mutants in Arabidopsis result in the early arrest of seed development, we investigated the role(s) of AtETHE1 by constitutively over-expressing the protein in plants. Over-expression of the native form of the protein had no observable effect on plant growth or development. However, over-expression of AtETHE1 in the cytosol results in plants that bolt faster, have a more upright growth habit, and set more seed.

We have shown that Arabidopsis Ethylmalonic Encephalopathy Protein 1 (AtETHE1), a GLX2-like protein with unknown function, is essential for seed development (Example 5) and shows enhanced expression in plants exposed to the abiotic stresses mannitol, NaCl, and abscisic acid (Example 4). In this Example, we demonstrate that constitutive over-expression of AtETHE1 results in plants that bolt sooner, have a more upright growth habit, and set more seed. Surprisingly, the enhanced growth properties are only observed when the protein is targeted to the cytosol, but not the mitochondrion, the normal location of AtETHE1. Without wishing to be bound by theory, these results suggest that the cytoplasmically localized protein catalyzes a reaction that may not normally occur in plant cells. Based on the phenotype of the transgenic plants we predict that the mis-localized ETHE1 may indirectly alter cytokinin levels.

Experimental Procedure: Materials and Methods

Plant Material and Growth Conditions

Studies in Arabidopsis thaliana Heyhn were performed on the Wassilewskija (WS) ecotype. Seeds were vernalized at 4° C. for 48 hours prior to being placed in a growth chamber. For measurement of toxicity and germination rates, seeds were surfaced sterilized with 70% ethanol for 30 seconds followed by 10% bleach for 30 minutes, rinsed three times with sterile deionized water and plated on half strength Murashige and Skoog (MS) plates solidified with 0.6% Gelrite and supplemented with 1.5% sucrose, pH 6.0. Plates were vernalized for four days at 4° C. and then placed in a controlled growth chamber at 23° C. on a 16:8 light/dark cycle.

Unless otherwise noted, plants were grown on commercial potting soil in a controlled growth chamber at 23° C. on a 16:8 hr light/dark cycle. The effect of over-expression of AtETHE1 on plant height, bolting rate, flowering time, and days to senescence was examined using 10 plants from each of four independent T₂ lines of AtETHE1₂₅₆-OE plants and from six independent T₂ lines of AtETHE1₂₉₄-OE plants. Plant height was measured from the base of the primary inflorescence. Bolting rate was measured as the days between germination and the first presence of the inflorescence. Flowering time was measured from germination to the day of the first open flower. Time to senescence was measured from germination to the first stage of senescence as defined by the yellowing of the rosette leaves. Four Arabidopsis AtETHE1₂₅₆-OE lines were analyzed for dry weight, number of siliques per plant, and number of seeds per silique. Dry weight was determined by cutting the base of the inflorescence at the soil line after the last flower had opened and drying the material allowing the material to dry fully between two papers for ten days in a 37° C. incubator. Weights of ten plants from each of the four transgenic lines were determined and compared with WT. Siliques were counted on ten individual plants of each transgenic line after the completion of flowering. The number of seeds per silique was determined under a dissecting microscope on five siliques from each of the ten plants per transgenic line.

Studies in tobacco were performed on the Petite Havana ecotype. Seeds were vernalized at 4° C. for 48 hours prior to being placed in a green house. Plants were grown on commercial potting soil with constant liquid feed and watered as needed. Seeds from six T₂ AtETHE1₂₅₆-OE transgenic lines were grown on soil for analysis of plant bolting rate, flowering time, seed yield, and days to senescence. Seed yield was determined by collecting seed pods over the lifetime of the plant and seed number determined by weight.

Arabidopsis (ecotype Landsberg Erecta) cell suspensions were grown in 50 ml liquid growth medium (1× Murashige and Skoog Basal Salts, 1× Gamborg's B5 vitamins, 3% (w/v) Sucrose, 0.59 g/L MES, 0.5 mg/L NAA, and 0.05 mg/L BAP, pH 5.7) at 25° C. with gentle agitation (130 rpm) in 16:8 hour light:dark cycles. A six ml aliquot was transferred to 50 ml fresh medium each week.

Generation of AtETHE1 Over-Expression Plants and Cell Cultures

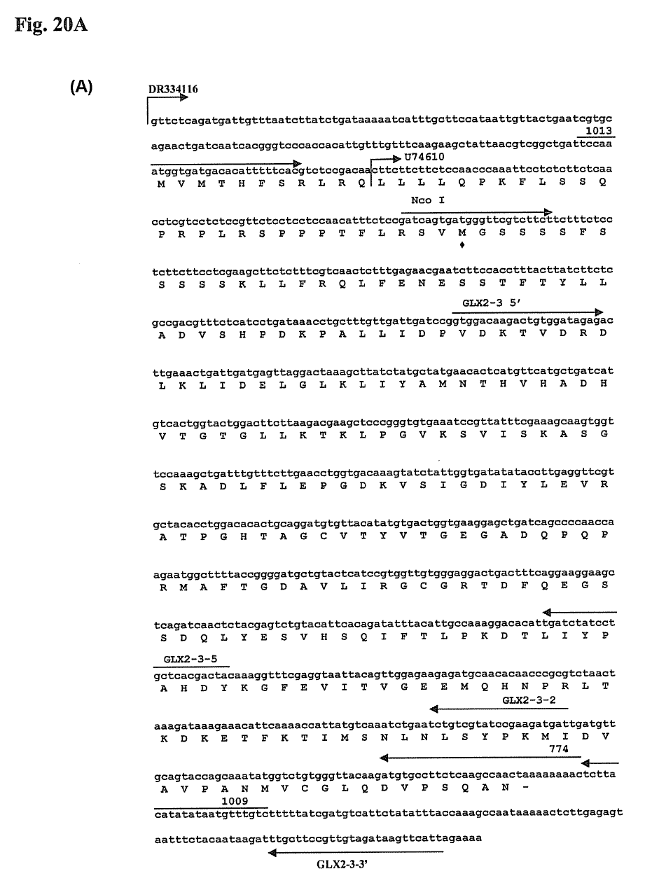

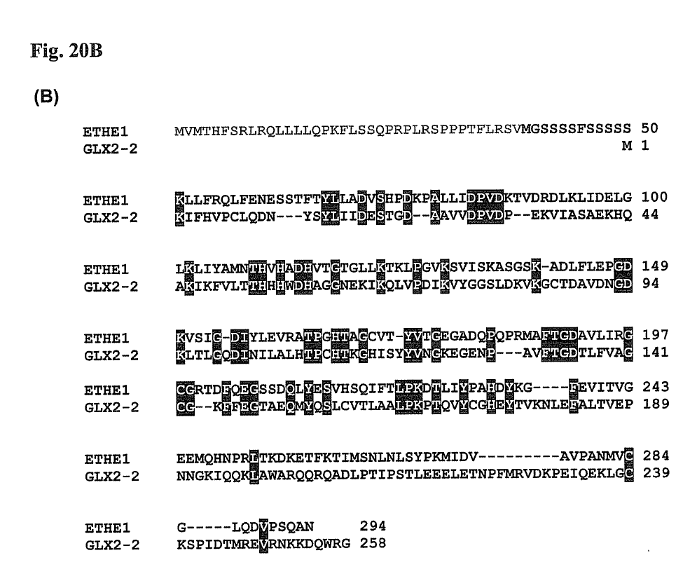

Based on the cDNA sequence of Arabidopsis ETHE1 (genebank id: 79606538) available at the time these experiments were initiated, a 35S over-expression construct of a 256 amino acid Arabidopsis ETHE1 protein (AtETHE1₂₅₆) was generated. A second larger AtETHE1 EST (gi:145362330) was subsequently identified and used to generate an over-expression construct that produced a 294 amino acid AtETHE1 protein (AtETHE1₂₉₄). Fragments containing AtETHE1₂₅₆ and AtETHE1₂₉₄ were generated by PCR with primers Nco1 and 2-3-3′ and 1013 and 2-3-3′, respectively, and cloned into pFGC 5941 (Genbank assession # AY310901) (FIG. 20A-B). After sequence confirmation, the constructs were transformed into Agrobacterium GV3101/PMP90 cells and used to transform wild-type plants (Clough, S. J. & Bent, A. F. (1998) Plant J. 16:735-743). Transgenic plants were identified by BASTA screening (1:4000 dilution) and the presence of the over-expression construct was confirmed by PCR with a vector specific primer and primer 2-3-2.

Over-expression constructs that produce AtETHE1₂₅₆ or AtETHE1₂₉₄ with C-terminal FAST tags were generated to facilitate subcellular localization and N-terminus determination studies (Ge, X., et al. (2005) EMBO Rep. 6, 282-288). AtETHE1 fragments were amplified from their respective pFGC clones using a pFGC vector specific promoter primer, and 774, a primer that eliminated the AtETHE1 stop codon, and cloned into pFAST (FIG. 20A). The constructs were sequenced and then transformed into GV3101/PMP90 Agrobacterium cells.

Wild-type Arabidopsis suspension cell cultures were transformed as follows: seven day old suspension cells were subdivided into fresh liquid growth medium at a high density (1:5 dilution) and allowed to rotate under normal conditions for 36 hours. The culture was further diluted (1:2) with fresh medium and allowed to rotate for an additional twelve hours. Agrobacterium cultures were grown to OD₆₀₀˜0.8, diluted (1:10) in fresh Arabidopsis liquid growth medium and washed three times in that same medium. The suspension cells were diluted (1:10) into a final volume of ten ml fresh medium in a 250 ml Erlenmeyer flask and 100 μl of the Agrobacterium dilution added. The cells were placed under normal lighting conditions with no shaking. After 48 hours, the cells were washed three times in fresh medium and resuspended in ten ml fresh medium containing 200 mg/L cefotaxime. The cells were allowed to rotate under normal conditions for three days and then plated on solid medium containing kanamycin (50 mg/L) and cefotaxime (200 mg/L). Positive transformants were confirmed by PCR screening using primers 557 and 255. Positive calli were reintroduced into liquid growth medium containing kanamycin (50 mg/L) and cefotaxime (200 mg/L) for localization and N-terminal determination experiments.

In order to test the effect of over-expression of AtETHE1 in tobacco, an EcoRI and XbaI fragment from ETHE1₂₅₆ PFGC5941 was transferred into pPZP111 (Hajdukiewicz, P., et al. (1994) Plant Mol. Biol. 25, 989-994). After sequence confirmation, the construct was transformed into Agrobacterium GV3101/PMP90 cells and used to transform wild-type Petite Havana tobacco plants through leaf disk infection (Dandekar, A. M., et al. (2005) Methods Mol. Biol. 286, 35-46). AtETHE1₂₅₆-OE/pPZP111 transformants were identified through kanamycin selection and confirmed by PCR using a vector specific primer and gene specific primer 2-3-2. The pPZP111 vector was transformed into separate plants as a control.

AtETHE1 RNA levels in wild-type and AtETHE1 over-expression plants were determined using RT-PCR on total RNA (1 μg) isolated from leaves using a Thermoscript RT-PCR kit. PCR was conducted for 25 cycles using the primer pair 2-3-5′ and 2-3-2 at 56° C. ACT8 was used as a control (An, Y. Q., et al. (1996) Plant J. 10, 107-121).

Wild-type Arabidopsis (Ws), AtETHE1₂₅₆-OE, and AtETHE1₂₉₄-OE seeds were sown onto half-strength MS plates with 0 mM to 2 mM valine, propionate, ethylmalonic acid, butyric acid, or crotonic acid (all purchased from Sigma). Neutralized, filter-sterilized metabolites were added after the medium was autoclaved. Once toxicity levels for wild-type seeds were obtained, wild-type, AtETHE1₂₅₆-OE, and AtETHE1₂₉₄-OE seeds were plated together on the same plates. Root lengths and cotyledon growth from 100 plants per stress were measured and compared after 7 days of growth.

Seedlings of wild-type and AtETHE1-OE lines were grown on MS-media plates for five days prior to infrared analysis. Seedlings were removed from the plates and washed extensively with distilled water for 15 min. The cotyledon and root were removed with a razor blade leaving the hypocotyls, which was dried on a glass slide at 37° C. for 40 min. Attenuated Total Reflectance (ATR) spectra were collected using a Harrick Split-pea ATR accessory interfaced to a Perkin Elmer 2000 Fourier transform infrared spectrometer. This accessory employs a single bounce Si (n=3.4) IRE and the standard deuterium triglycine sulfate (DTGS) detector on the Spectrum 2000 macro bench. The IRE is 3 mm in diameter with an infrared active region of 200 μm in diameter. The samples were brought into intimate contact with the IRE using a load of 0.5 kg. Spectra collected represent the average of 32 individual scans with a spectral resolution of 4 cm⁻¹. Spectra was collected in triplicate for 3 plants of each line

Microscopy

Morphology of the vascular tissue in AtETHE1₂₅₆-OE and wild-type plants was analyzed by light microscopy. Sections (1 cm) of the primary inflorescence from Arabidopsis plants at stage three (as defined by Altamura, M. M., et al. (2001) New Phytologist. 151, 381-389), three cm from the base, were cut for analysis on four plants from each of four transgenic AtETHE1₂₅₆-OE lines and 23 wild type plants. Sections were fixed in FAA (formalin:acetic acid:acetone 10:5:50) for 16 hours under vacuum, rinsed twice with ddH₂O, one hour each, and dehydrated under a graded ethanol series: 20%, 30%, 50%, 70%, 95%, 100% for at least an hour each. Samples were left in 100% ethanol for further analysis.

Cross sections of 50 μM relative thickness were generated on a Vibratome Series 1000 sectioning system and stored in distilled water. Cross sections were stained in 0.1% Toluidine blue and mounted in a semi-permanent gelatin-glycerine mount (10 g non-flavored gelatin, 150 ml glycerine, 0.1 g sodium salycilate, 170 ml ddH₂O). Images were observed using light microscopy on an Olympus IX-81 fluorescence deconvolution microscope system. Data were analyzed with Image Pro Plus (Media Cybernetics) and organized with Photoshop. Statistical analysis of variance was performed using the two sample t-test assuming unequal variances (Excel).

N-Terminal Analysis and Protein Localization

The locations of AtETHE1₂₉₄ and AtETHE1₂₅₆ were determined by subcellular fractionation of pFAST transgenic cell cultures using differential centrifugation. Harvested cells were lysed in isolation buffer (0.35 M Sorbitol, 25 mM MOPS, 0.1% BSA, 2 mM EDTA, 0.1% BME, 1% PVP-40) using a blender (2 g/5 ml buffer) and centrifuged at 2500×g for 5 minutes to remove the chloroplasts and cellular debris. The supernatant was then centrifuged at 15000×g for 15 minutes to pellet the mitochondria. The mitochondrial pellet was washed three times in wash buffer (0.4 M Sucrose, 2 mM EDTA, 10 mM MOPS, 0.1% BSA) using alternating low and high speed spins. The mitochondrial pellet was resuspended in lysis buffer (50 mM TRIS pH 8.0, 0.5% Triton X-100) and placed on ice for 30 minutes. The lysed mitochondrial fraction was centrifuged for 30 minutes at 12000×g. Protein was quantitated using a BCA assay (PIERCE), and ten μg was loaded onto an SDS-PAGE gel for western detection using anti-FLAG antibody (Sigma). Mitochondrial cytochrome c oxidase was used as a control to monitor the purity of the fractions.

Cytosolic or mitochondrial fractions from cultures over-expressing AtETHE1₂₅₆FLAG or AtETHE1₂₉₄FLAG, respectively, were subjected to affinity-purification using anti-FLAG chromatography (Sigma) for N-terminal determination. Purified AtETHE1 was eluted using FLAG peptide (Sigma F3290) and analyzed by SDS-PAGE gel and western blotting. Purified AtETHE1 proteins were further resolved by SDS-PAGE. Gel slices containing the proteins were digested using trypsin (Sigma) and GluC (pH 7, Pierce), and the resulting peptides were analyzed on a MALDI-TOF mass spectrometer (Bruker Reflex III) in positive ion mode essentially as described (Tiranti, V., et al. (2004) Am. J. Hum. Genet. 74, 239-252). Mass spectral data were analyzed using the PAWS program at http://prowl.rockefeller.edu.

Results and Discussion:

Localization and N-Terminal Determination Studies

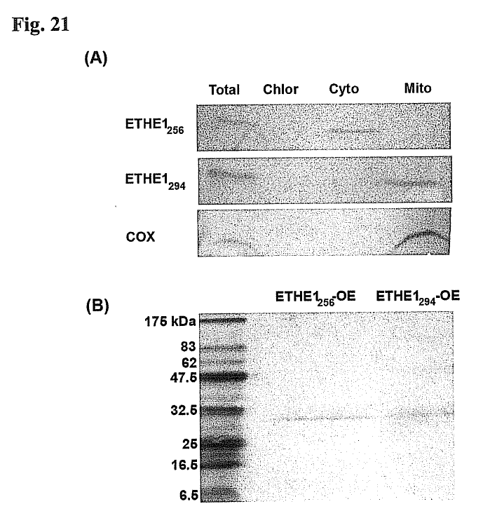

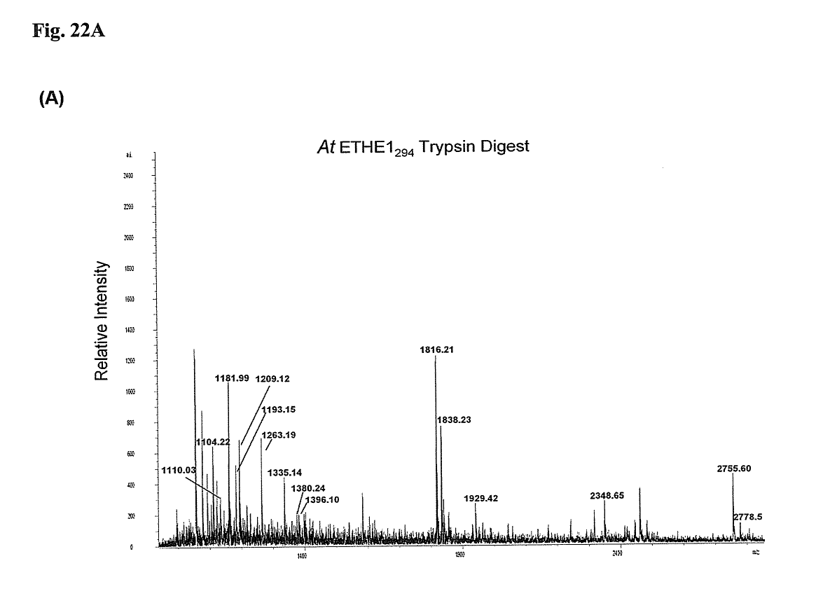

Arabidopsis ETHE1 was previously reported as a 256 amino acid (27.7 kDa) predicted mitochondrial protein showing similarity to GLX2 (Maiti, M. K., et al. (1997) Plant Mol. Biol. 35, 471-481). Consistent with this prediction, a sequence alignment of Arabidopsis ETHE1 with the cytosolic isozyme of Arabidopsis GLX2 showed that AtETHE1 contains an N-terminal extension, which resembles a mitochondrial leader peptide (FIG. 20A-B). To determine if AtETHE1 is in fact localized in the mitochondrion and to determine the AtETHE1 N-terminal processing site, a construct based on the reported AtETHE1 cDNA (ETHE1₂₅₆) was generated in the pFAST expression vector. The construct, which expresses AtETHE1₂₅₆ with a C-terminal FLAG tag, was transformed into Arabidopsis suspension cell cultures. Fractionation studies were performed on the transgenic cell culture followed by western blot analysis using anti-FLAG antibody to determine the subcellular location of AtETHE1₂₅₆. In contrast to what was expected, the fractionation study showed that AtETHE1₂₅₆ is clearly cytosolic (FIG. 21A). Duplicate blots were probed with an antibody to the mitochondrial cytochrome c oxidase as a control. As expected, cytochrome c oxidase was found in the total protein and mitochondrial fractions (FIG. 21A). It was not detected in the cytosolic fraction. Our finding that Arabidopsis ETHE1₂₅₆ is present in the cytosol is inconsistent with a previous observation that human ETHE1 is a mitochondrial protein, suggesting that AtETHE1₂₅₆ may not represent the full-length protein.

A second Arabidopsis ETHE1 cDNA was subsequently identified that encodes a protein with an additional 38 N-terminal amino acids. This cDNA, which we confirmed as full-length through RT-PCR, has the potential to encode a protein of 294 amino acids with a molecular weight of 32.3 kDa (FIG. 20B). Therefore, transgenic cell cultures expressing the full-length, AtETHE1₂₉₄ protein in the pFAST expression vector were generated and subjected to the same fractionation studies as described above. As expected, AtETHE1₂₉₄ was found in the total protein and mitochondrial fractions (FIG. 21A). It was not detected in the cytosol. Interestingly, FAST-tagged proteins of approximately 28 kDa were identified in both AtETHE1₂₅₆ FLAG and AtETHE1₂₉₄ FLAG cells suggesting that the N-terminal processing site of AtETHE1₂₉₄ is near the N-terminus of AtETHE1₂₅₆ (FIG. 21B).

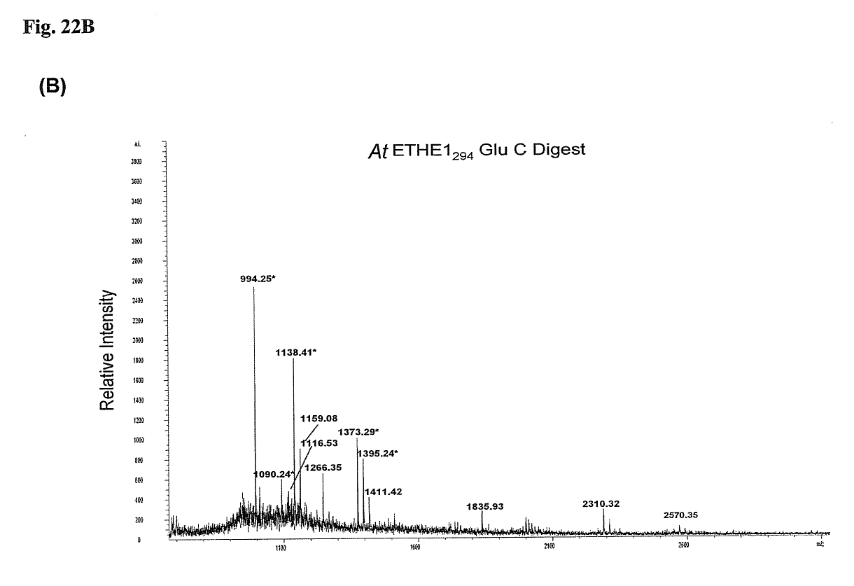

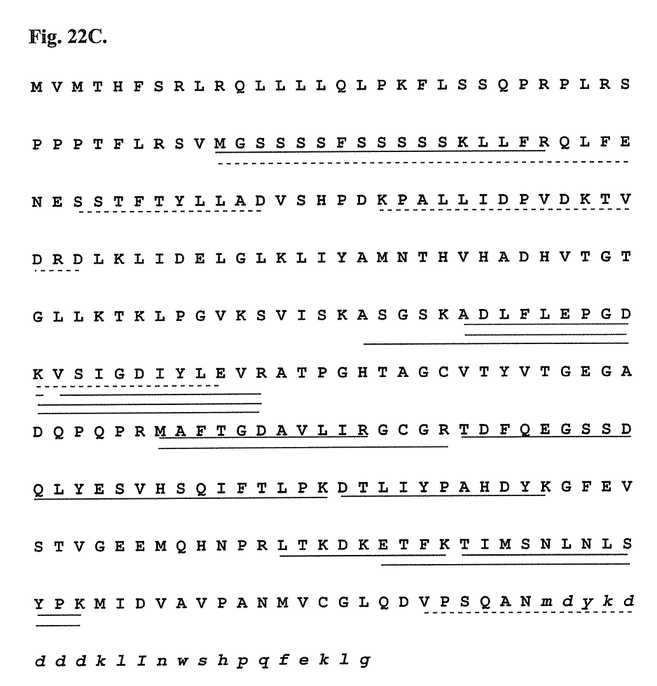

Although it was previously shown that human ETHE1 is localized to the mitochondrion, the mature N-terminus of an ETHE1 protein is not known in any organism. Therefore, we purified the AtETHE1₂₅₆ FLAG and AtETHE1₂₉₄ FLAG proteins using FLAG affinity chromatography and subjected them to N-terminal analysis. Several attempts of N-terminal sequencing proved unsuccessful, likely due to an N-terminal block. Therefore, we mapped the N-terminal sequences of the proteins using peptide mapping with the endopeptidases trypsin and GluC. Peptide fragments were analyzed using MALDI-TOF mass spectrometry and the data compiled from the peak lists of both endopeptidase cleavages were mapped to the AtETHE1 sequence using PAWS (FIG. 22A-C, Table 3). The peptides produced good coverage and showed several overlapping peptides from the various cleavages allowing an accurate prediction of the processed form of AtETHE1 (FIG. 22C). Peptides obtained from AtETHE1₂₅₆ were observed beginning at the methionine located at amino acid 39 of the full-length form of the protein (data not shown). This corresponds to the unprocessed form of the protein.

TABLE 3
Peptide matches of ETHE1₂₉₄ purified protein.
ETHE1₂₉₄ Peptide Masses

| | | | Position | |
|---|---|---|---|---|
| | Experimental | | of | |
| peptide # | mass | error | peptide | Sequence |
| 1 | 1796.23 | −1.37 | 39-56 | MGSSSSFSSSSSKLLFRa |
| | | | | (SEQ ID NO: 24) |
| 2 | 2310.32 | 1.8 | 39-60 | MGSSSSFSSSSSKLLFRQLFE |
| | | | | (SEQ ID NO: 25) |
| 3 | 1116.53 | −0.04 | 63-72 | SSTFTYLLAD |
| | | | | (SEQ ID NO: 26) |
| 4 | 1793.93 | 1.06 | 78-93 | KPALLIDPVDKTDVDRDb |
| | | | | (SEQ ID NO: 27) |
| 5 | 2778.58 | 0.86 | 136-161 | ASGSKALDFLEPGDKRSIGDIVLER |
| | | | | (SEQ ID NO: 28) |
| 6 | 1104.22 | 0.33 | 142-151 | ADLFLEPGDK |
| | | | | (SEQ ID NO: 29) |
| 7 | 2348.65 | 0.58 | 142-162 | ADLFLEPGDKRSIGDIVLEVR |
| | | | | (SEQ ID NO: 30) |
| 8 | 1137.08 | −0.47 | 151-161 | KVSIGDIYLE^{c} |
| | | | | (SEQ ID NO: 31) |
| 9 | 1263.19 | 0.5 | 152-162 | VSIGDIVLEVR |
| | | | | (SEQ ID NO: 32) |
| 10 | 1193.15 | 0.51 | 187-197 | MAFTGDAVLIR |
| | | | | (SEQ ID NO: 33) |
| 10 | 1193.12 | 0.48 | 187-197 | MAFTGDAVLIR^{b} |
| | | | | (SEQ ID NO: 34) |
| 11 | 1165.99 | 0.49 | 187-201 | MAFTGDAVLIRGCGR^{b} |
| | | | | (SEQ ID NO: 35) |
| 12 | 2755.6 | 0.7 | 202-225 | TDFQEGSSDQLESVHSQIFTLPK |
| | | | | (SEQ ID NO: 36) |
| 13 | 1335.14 | 0.51 | 226-236 | DTLIVPAHDYK |
| | | | | (SEQ ID NO: 37) |
| 14 | 1110.08 | −0.47 | 253-261 | LTKDKETFK |
| | | | | (SEQ ID NO: 38) |
| 15 | 1110.03 | −0.42 | 253-261 | LTKDKETFK^{c} |
| | | | | (SEQ ID NO: 39) |
| 16 | 1887.42 | −1.45 | 258-273 | ETFKTIMSNLNLSYPK^{a} |
| | | | | (SEQ ID NO: 40) |
| 17 | 1380.24 | 0.61 | 262-273 | TIMSNLNLSYPK |
| | | | | (SEQ ID NO: 41) |
| 18 | 1380.1 | 0.61 | 262-273 | TIMSNLNLSYPK^{b} |
| | | | | (SEQ ID NO: 42) |
| 19 | 1266.35 | 0.34 | 289-299 | VPSQANmdykd |
| | | | | (SEQ ID NO: 43) |

^{a}= aceylated, ^{b}= oxidated, ^{c}= sodiated. Masses obtained from MALDI-TOF analysis were matched to AtETHE1 using PAWS. Peptide sequences unbolded refer to peptides matched to the protein sequence of AtETHE1 after digestion by trypsin. Peptide sequences bolded refer to matched peptides from Glu C digestion. The position of the peptides correspond to the amino acid numbering of AtETHE1₂₉₄.

Localization prediction programs (Mitoprot-2, TargetP) predicted two possible N-terminal processing sites for AtETHE1₂₉₄, the first at proline 31 and the second at lysine 52 of the full length protein. Both of these sites would produce endopeptidase cleavage fragments with masses within range of the MALDI-TOF detection. AtETHE1₂₉₄ processed at P₃₁ would produce trypsin and GluC fragments of 826.47 Da. and 3305.67 Da., respectively. A processing site at K₅₂ of AtETHE1₂₉₄ would not produce a fragment detectable by trypsin digest; however, it would produce a fragment with a mass of 1192.69 after GluC digestion. The majority of the endopeptidase digestion peaks found in the MALDI-TOF spectra were identified and produced good sequence coverage, yet, surprisingly none of the masses could be detected in MALDI-TOF spectra associated with either of the two predicted processing sites (FIG. 22A-C, Table 3). However, two peak masses that correspond to overlapping fragments in the two endopeptidase digestions: a trypsin digest mass of 1796.23 Da. and a GluC digest mass of 2310.32 Da., corresponding to the peptides M₃₉-R₅₆ and M₃₉-E₆₀, respectively were identified (FIG. 22A-B, Table 3). Therefore, mapping analysis of AtETHE1₂₉₄ indicated a leader sequence processing site at the methionine located at position 39 of the full length protein (Table 3).

It should be noted that while M₃₉ was not initially predicted as a cleavage site, it is still consistent will all the sequence features necessary for the Mitochondrial Processing Peptidase (MPP) recognition and activity. Specifically, AtETHE1 contains an Arg at the −3 position and an additional distal basic residue at the −11 position. MPP has also been shown to exhibit a preference for aromatic amino acids and, to a lesser extent, hydrophobic amino acids at position 1, which is also consistent with the processing site at M₃₉ of AtETHE1. In addition, the M₃₉ processing site contains several hydrophillic/hydroxylamino acids at positions −2 and −3 which should enhance the activity of APP.

These results demonstrate that Arabidopsis ETHE1 is actually larger than we previously predicted (Maiti, M. K., et al. (1997) Molecular Characterization of Glyoxalase II from Arabidopsis Thaliana. Plant Mol. Biol. 35, 471-481) and contains a 38 amino acid leader peptide that is essential for the protein's localization within the mitochondrion. There are several observations that strengthen our confidence in the result: (1) we are able to identify peptides in both the trypsin and GluC digests corresponding to an N-terminal peptide beginning with M₃₉; (2) similar peptide maps were obtained for the purified AtETHE1₂₅₆ and AtETHE1₂₉₄ proteins; and (3) this processing site is also consistent with the observed molecular weight seen in the western blot (FIG. 21B).

Generation of AtETHE1 Over-Expressing Arabidopsis Plants

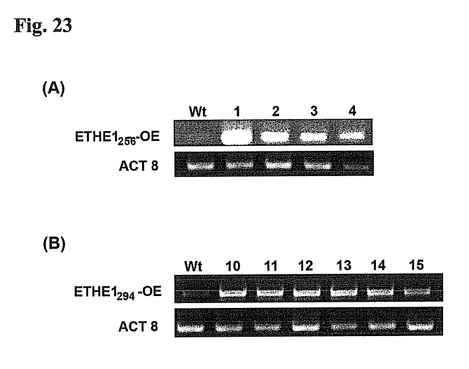

Plants that over-express either AtETHE1₂₅₆ or AtETHE1₂₉₄ were generated in order to investigate possible functional roles of AtETHE1 in plant growth and development and to determine if over-expression of AtETHE1 can enhance the growth properties of plants by providing stress resistance. Over-expression constructs of both AtETHE1₂₅₆ and AtETHE1₂₉₄ driven by the constitutive 35S promoter were generated and transformed into wild-type Arabidopsis (Ws) plants. Four independent Basta-resistant AtETHE1₂₅₆ transgenic lines and six independent Basta-resistant AtETHE1₂₉₄ transgenic lines were selected and confirmed by PCR. RT-PCR was performed on the individual lines and all of the transgenic plants showed significantly increased levels of AtETHE1 transcript in comparison to wild type plants (FIG. 23 A-B).

AtETHE1₂₅₆-OE Plants Show Resistance to High Concentrations of Valine

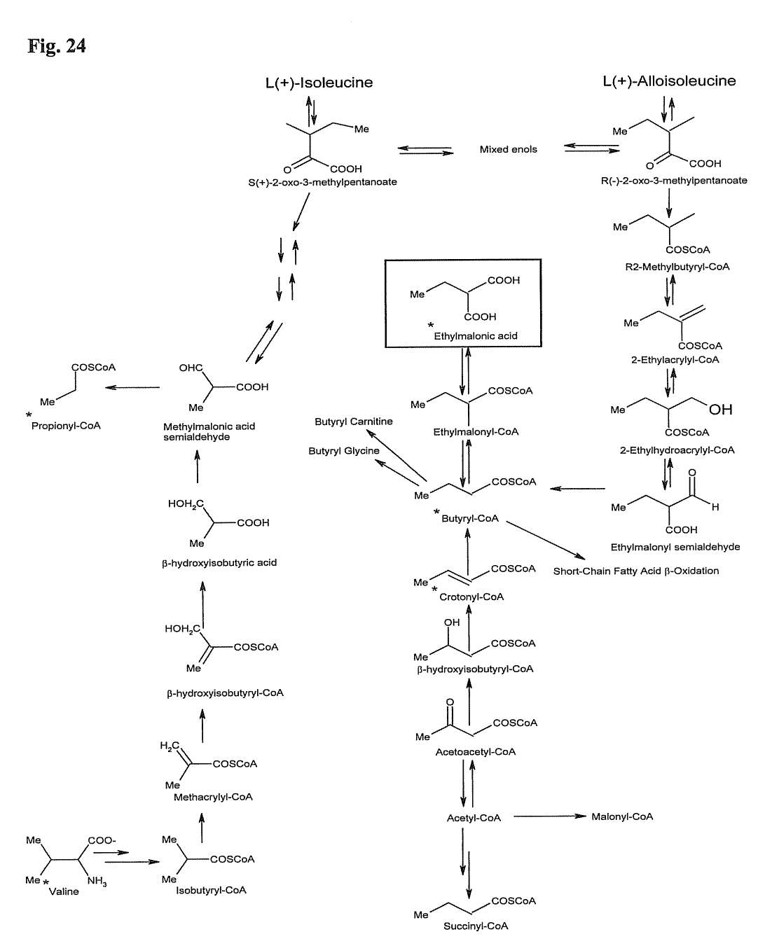

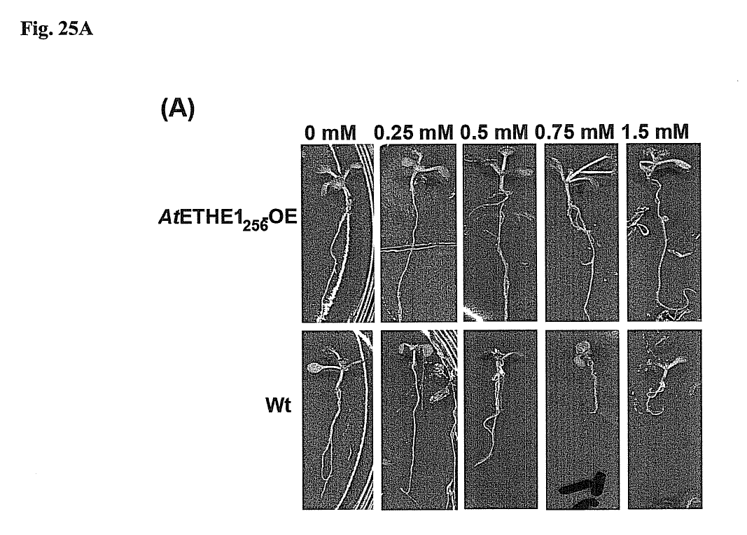

While the biochemical role of ETHE1 is not known in any organism, physiological changes observed in patients with EE have been used to predict potential biochemical roles for the protein. Mutations in human ETHE1 result in elevated levels of C₄ and C₅ plasma acylcarnitines and markedly elevated urinary excretion of ethylmalonic acid, and C₄₋₆ acylglycines. Ethylmalonic acid is primarily derived from the carboxylation of butyryl-CoA, which is derived from the β-oxidation of short chain fatty acids, or from 2-ethylmalonic-semialdehyde, the final product of the R-pathway for the catabolism of isoleucine (FIG. 24). This raised the possibility that human ETHE1 and by analogy Arabidopsis ETHE1 may be involved in the removal of a hydroxyacid or CoA ester that is faulted as part of amino acid and/or lipid metabolism. We predicted that if AtETHE1 is actually involved in the detoxification of byproducts from amino acid metabolism, then over-expression of the enzyme may eliminate the toxic effects observed when plants are grown in the presence of high levels of certain amino acids or their toxic intermediates. In order to test this, we examined the sensitivity of AtETHE1-OE lines to valine, propionate, ethylmalonic acid, butyric acid, and crotonic acid (FIG. 24).

No differences were observed between AtETHE1₂₉₄-OE and wild-type plants at the various concentrations tested for the different metabolites (data not shown). These results suggest that AtETHE1 may not normally be involved in the removal of toxic metabolites of branched chain amino acid metabolism or the β-oxidation of fatty acids. However, these results also do not rule out this possibility.

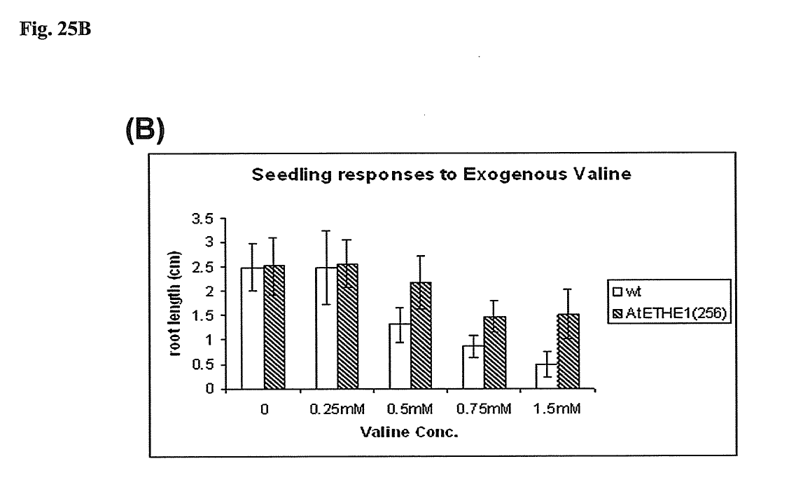

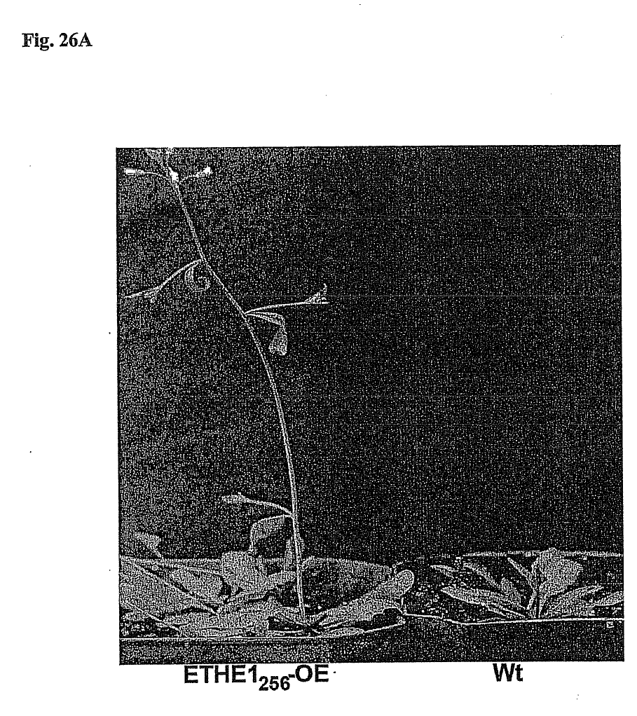

Even though no differences were observed in plants over-expressing the full-length, AtETHE1₂₉₄ protein, AtETHE1₂₅₆-OE plants did exhibit growth differences on plates containing exogenous valine (FIG. 25A-B). Valine concentrations of 1.5 mM and greater are toxic to wild-type plants, inhibiting root growth by approximately 80% over 7 days of growth. The same level of valine only inhibited the growth of AtETHE1₂₅₆-OE plants by about 40% (FIG. 25A-B). However, in contrast to our predictions, AtETHE₂₅₆-OE plants displayed the same sensitivity to propionate, ethylmalonic acid, butyric acid, and crotonic acid as wild-type plants (data not shown).

Mutations in acetolactate synthase (ALS), an enzyme that catalyses the first common step of branched chain amino acid biosynthesis can confer resistance to high levels of valine. ALS is regulated through feedback inhibition by the end products of the pathway. Complete inhibition of ALS leads to plant death primarily through starvation for essential amino acids; however single point mutations that reduce the binding affinity of ALS to its inhibitors have been reported that have little effect on its native enzymatic function, while providing plants with resistance to exogenous sources of valine.

Exogenous valine toxicity can also arise from methylacrylyl-CoA, a toxic degradation product, which can accumulate in the presence of high concentrations of valine. There is also evidence for the production of methylmalonate in patients compromised in methylmalonic semialdehyde dehydrogenase which catalyses the conversion of methylmalonate semialdehyde to propionyl-CoA. Methylmalonate is a known toxin whose toxic effects have been studied extensively in patients displaying methylmalonic aciduria. Preliminary studies have shown that methylmalonate is also toxic in plants. Given the similarity between methylmalonate and ethylmalonate and the excretion of ethylmalonic acid observed in EE patients, it is possible that ETHE1 be directly/indirectly involved in the hydrolyzing methylmalonate or one of its precursors, which in turn could promote the increased resistance of exogenous valine. However, interestingly, it appears that high levels of cytoplasmic AtETHE1 can confer resistance to inhibitory levels of valine, while the mitochondrial protein can not.

Over-Expression of AtETHE1₂₅₆ Leads to Earlier Bolting and Flowering in Arabidopsis

Plants that over-express cytosolic, AtETHE1₂₅₆, and mitochondrial, AtETHE1₂₉₄, display drastically different phenotypes. AtETHE1₂₉₄-OE plants appear normal in every respect. At this time we are unable to identify any specific effects of over-expressing AtETHE1₂₉₄. In contrast, plants that over-express AtETHE1₂₅₆ display several changes in their growth properties.

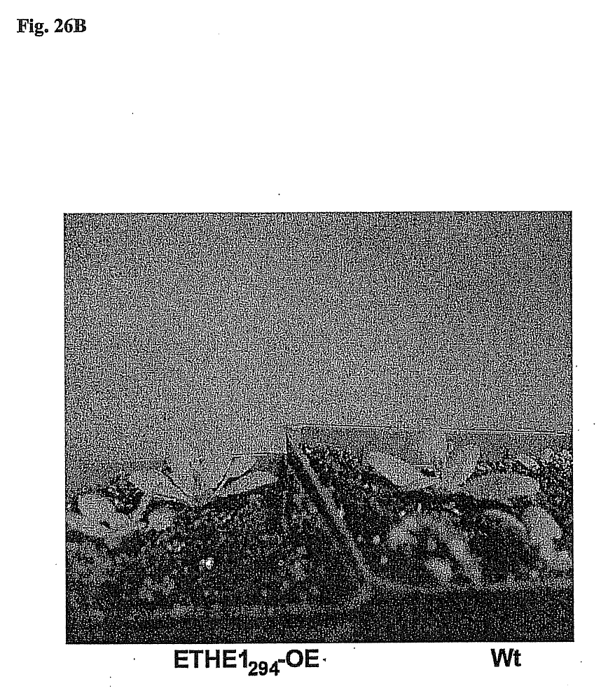

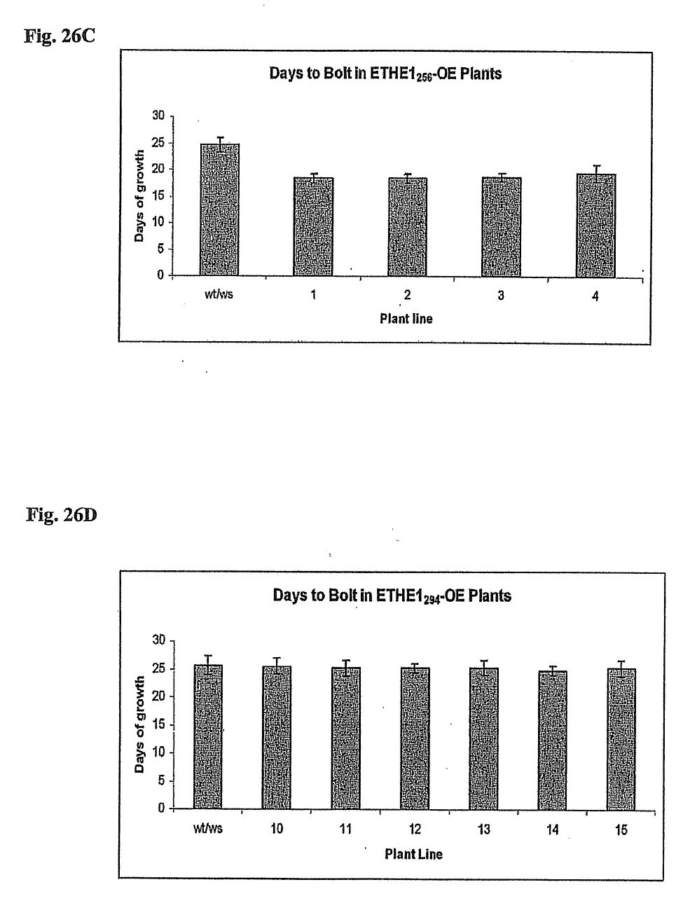

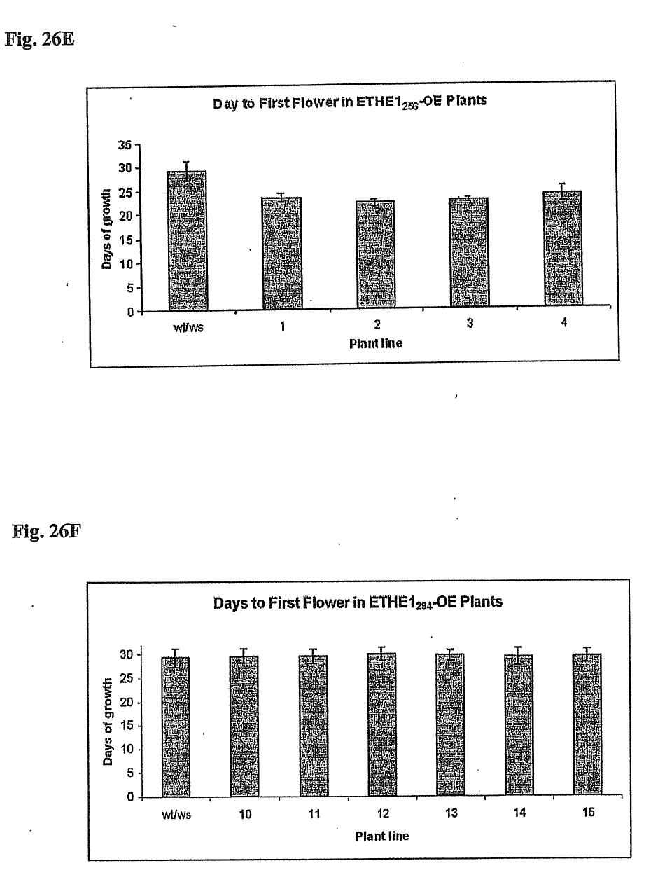

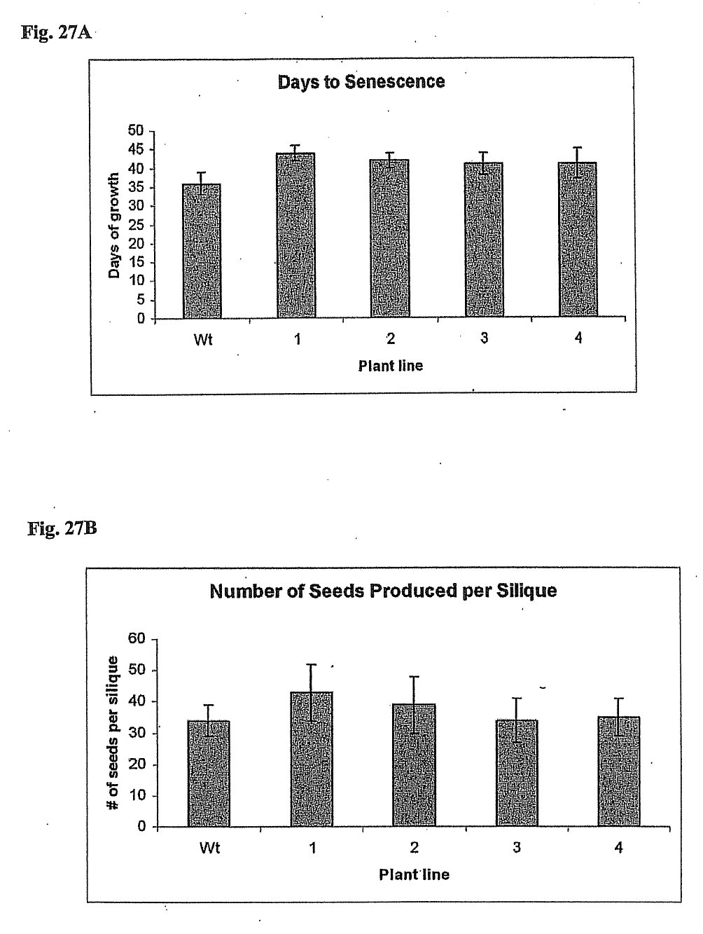

The phenotypes described below were observed over three generations and in several independent transgenic lines, indicating the changes are due to the over-expression of AtETHE1₂₅₆ and are relatively stable. Wild-type plants and AtETHE1₂₅₆-OE transgenic seedlings grown on solid MS growth medium for fourteen days did not exhibit any noticeable differences. Likewise no differences were observed between the two lines during the first eighteen days of growth in soil (data not shown). However AtETHE1₂₅₆-OE plants bolted significantly earlier than wild-type plants grown under the same conditions (FIG. 26A-C). AtETHE1₂₅₆-OE plants typically bolted 19 days (+/−1) after germination while wild-type and AtETHE1₂₉₄-OE plants did not bolt until day 24 (+/−1) (FIG. 26A-D). Consistent with the earlier bolting time, AtETHE1₂₅₆-OE plants also flowered significantly (P<0.5) earlier when compared to wild-type plants (FIG. 26E). In contrast to wild-type and AtETHE1₂₉₄-OE plants that typically flowered 29 days (+/−2) after germination, AtETHE1₂₅₆-OE plants flowered on average 23 (+/−1) days after germination (FIG. 26E-F).

The timing of flowering is an important developmental event that contributes to crop productivity, especially in regions with short growing seasons. If a plant moves from vegetative growth into its reproductive stage too early, seed yield can be limited due to an inadequate supply of energy from the reduced number of roots and leaves. Alternately, if the plant moves too slowly from its vegetative state to reproductive growth, it may not have enough time to produce mature seeds. There are several cues that guide the transition from vegetative to reproductive growth, including environmental cues such as photoperiod and vernalization, as well as phytohormonal cues, such as abscisic acid (ABA), cytokinin (CK) and gibberellin (GA) levels (reviewed in (56). ABA is generally described as an inhibitor of flowering, which is consistent with the early flowering phenotype of Arabidopsis mutants that are deficient in or insensitive to ABA. CKs influence cell division and shoot formation, and are classified as promoters of flowering. Transgenic plants deficient in CKs flower late, whereas plants that are enriched in CKs have been shown to flower early. Exogenous treatment with GAs has also been shown to cause flower formation and GA-deficient and insensitive mutants of Arabidopsis provide evidence that GAs are required for flowering under short days. The early bolting and flowering observed in AtETHE1₂₅₆-OE plants therefore could result from elevated levels of either CKs or GA. Without wishing to be bound by theory, it is though that the observation that AtETHE1₂₅₆ but not AtETHE1₂₉₄ can promote the earlier flowering suggests that the cytoplasmic farm of AtETHE1 may catalyze a reaction that is different from that of the native, mitochondrially localized enzyme, or that it acts on a substrate that is not accessible to the mitochondrial enzyme.

AtETHE1₂₅₆ Expression Enhances Seed Yield in Arabidopsis

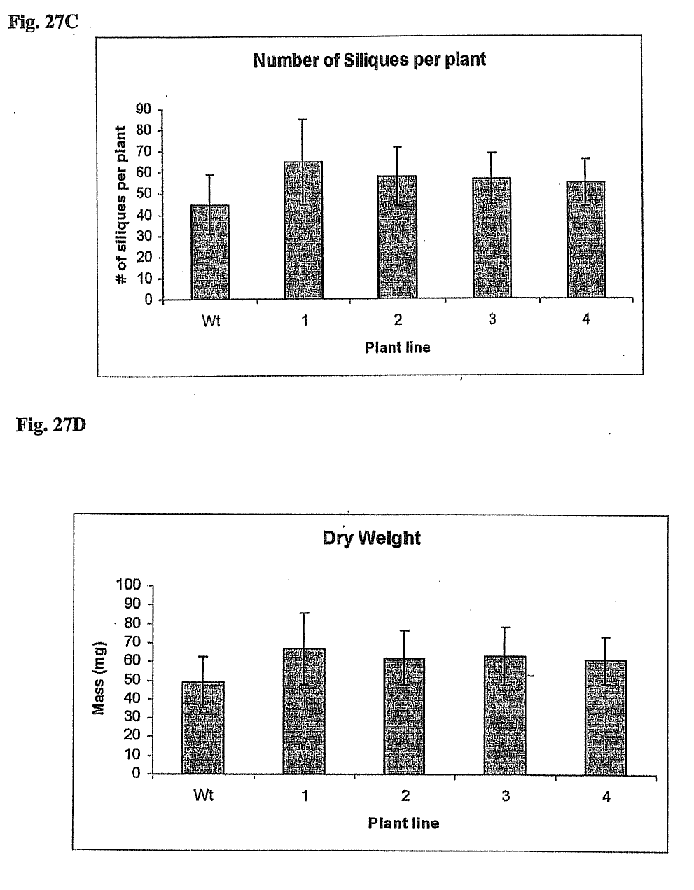

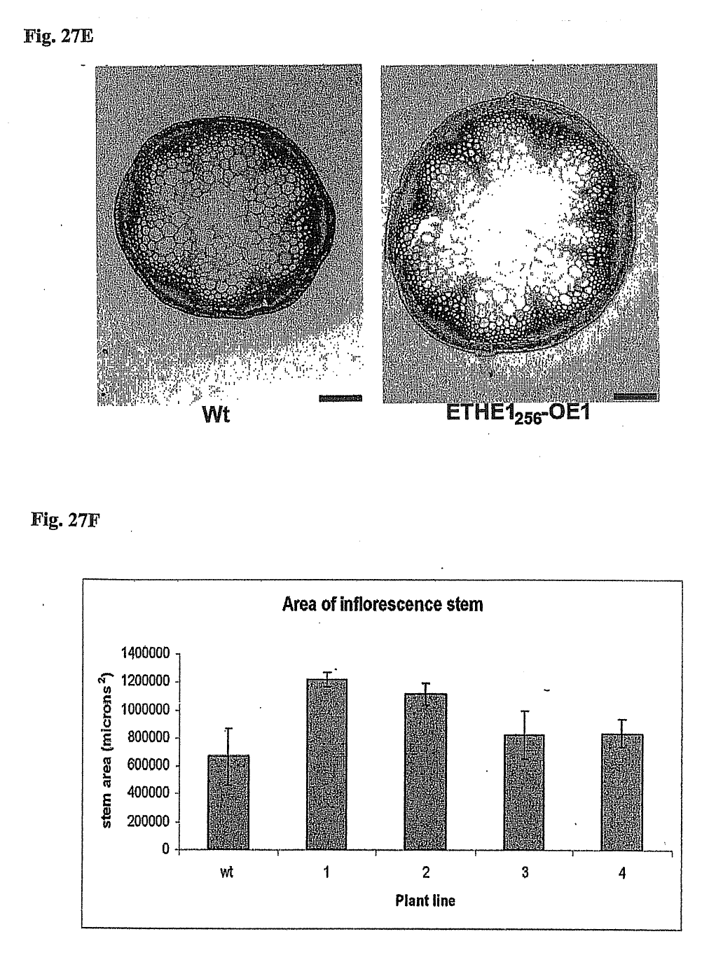

In addition to flowering earlier than wild-type plants, AtETHE1₂₅₆-OE plants also exhibited delayed senescence, flowering 6 days longer than wild-type plants (FIG. 27A). AtETHE1₂₅₆-OE plants typically stopped flowering 42 (+/−3) days after germination. In contrast, wild-type plants stopped flowering 36 (+/−3) days after germination. In the absence of environmental cues such as osmotic stress or pathogenic attack, the onset of senescence is generally determined by the age of the plant; however, this process, similar to the onset of flowering, can be influenced by the levels of certain phytohormones. The most widely known hormones involved in controlling senescence are CKs. Studies have shown delayed senescence upon the application of CKs as well in transgenic plants overproducing a key enzyme of cytokinin biosynthesis, isopentenyl transferase (ipt). Without wishing to be bound by theory, the delayed senescence phenotype of AtETHE1₂₅₆-OE plants along with the early bolting and flowering suggests that AtETHE1₂₅₆-OE plants may contain increased endogenous CK levels.

Because AtETHE1₂₅₆-OE plants display both earlier and longer flowering times compared with wild-type plants, we predicted that they would have a greater seed yield as well. The number of seeds per individual silique, and the number of siliques each plant produced were measured on plants from the individual transgenic lines and compared with wild-type plants (FIG. 27B-D). No statistically significant differences were observed in the number of seeds per silique produced between wild-type and AtETHE1₂₅₆-OE plants (FIG. 27B). It should be noted that while AtETHE1₂₅₆-OE line 1 appears to show an increase in the number of seeds produced per silique (42+/−12) in comparison to wild-type (36+/−6) a statistical t-test did not show a significant difference (P<0.05). A significant difference was however observed in the number of siliques produced in AtETHE1₂₅₆-OE plants compared with wild-type plants (FIG. 27C). Consistent with an increase in the duration of flowering, AtETHE1₂₅₆-OE plants produced on average 31% more siliques than wild-type plants (AtETHE1₂₅₆-OE 59 siliques: wild-type 45 siliques) (FIG. 27C). These results demonstrate that high level expression of AtETHE1₂₅₆ in the cytoplasm increases the reproductive life time of the plant and overall seed yield.

Arabidopsis ETHE1₂₅₆-OE Plants have Thicker Primary Inflorescence Stems

Arabidopsis plants expressing AtETHE1₂₅₆ are taller and also display thicker, more upright primary inflorescence stems than wild-type plants at the same stage of growth. Consistent with this observation we found an overall increase in dry weight of 24% in the AtETHE1₂₅₆-OE plants (AtETHE1₂₅₆-OE 60 mg per plant: wild-type 49 mg) (FIG. 27D). The growth habit of AtETHE1₂₅₆-OE plants was investigated further by examining cross-sections of the basal region of the primary inflorescence of both wild-type and AtETHE1₂₅₆-OE transgenic plants at the same stage of growth (FIG. 27E). Analysis of cross-sections demonstrated a significant difference in the area of the primary inflorescence between plants over-expressing AtETHE1₂₅₆ and wild-type plants (FIG. 27E). AtETHE1₂₅₆-OE plants were found to have on average a 28% increase in stem area compared with wild-type plants (AtETHE1₂₅₆-OE: 920.8 mm²; wild-type: 666.1 mm²) (FIG. 27F). Upon closer examination, no significant differences in the interfasicular fibers or the number of vascular bundles were observed between the AtETHE1₂₅₆-OE and wild-type plants (data not shown). The increase in the area of the primary inflorescence observed in plants expressing AtETHE1₂₅₆-OE appears to be primarily a result of cell expansion in the central parenchyma (FIG. 27E). The parenchyma cells are involved in photosynthesis, storage, secretion, movement of water, and transport of food depending on their localization within the plant body and therefore, expansion and enlargement of these cells could potentially provide enhanced growth properties.

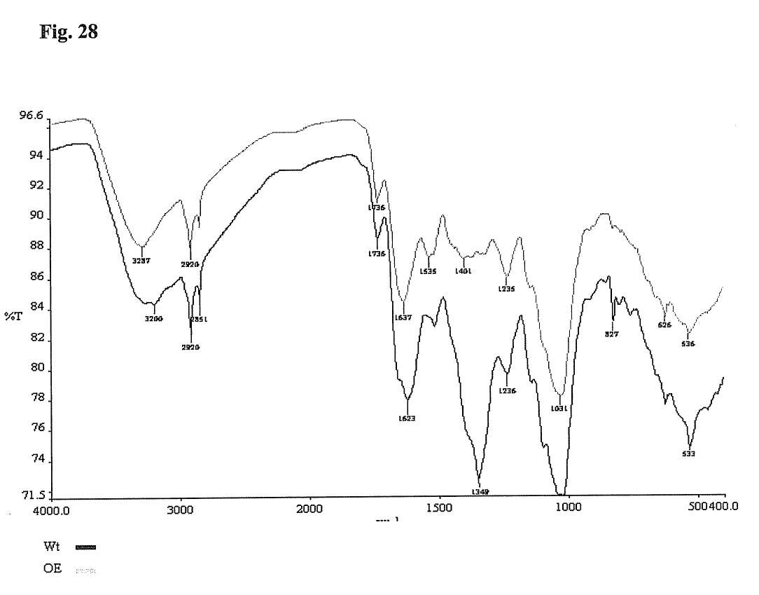

Because of the changes observed in AtETHE1-OE plants, it was hypothesized that there should be chemical differences between AtETHE1-OE₂₅₆ and wild-type plants. Infrared Spectroscopy was therefore used to generate a spectral map of stems because of its ease of use and a library of known spectra. Transmission spectra of wild type and AtETHE1-OE₂₅₆ seedling hypocotyls shown in FIG. 28 represent an average of 3 plants from each line and 32 scans per line. Comparison of the two spectra show a high level of similarity with the exception of two peaks centered at 1349 cm⁻¹ and 827 cm⁻¹ which are present in wild-type seedling hypocotyls but are absent in AtETHE1-OE seedling hypocotyls. These two peaks were run against a library of infrared spectra and appear to correspond to a nitrate. This raises the possibility that the AtETHE1-OE₂₅₆ lines utilize nitrate at a higher rate.

AtETHE1₂₅₆ Over-Expression in Nicotiana tabacum Results in Enhanced Growth Properties

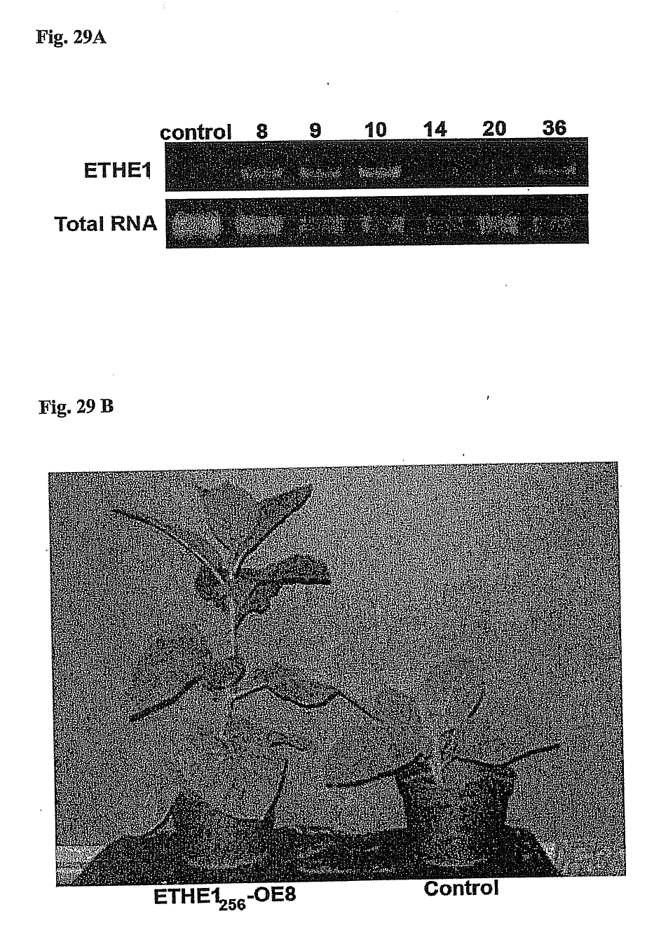

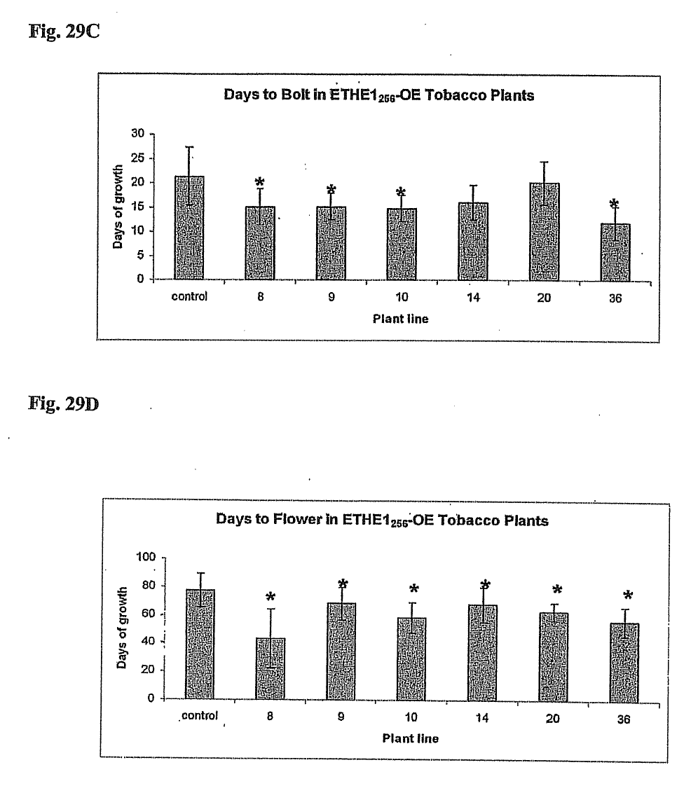

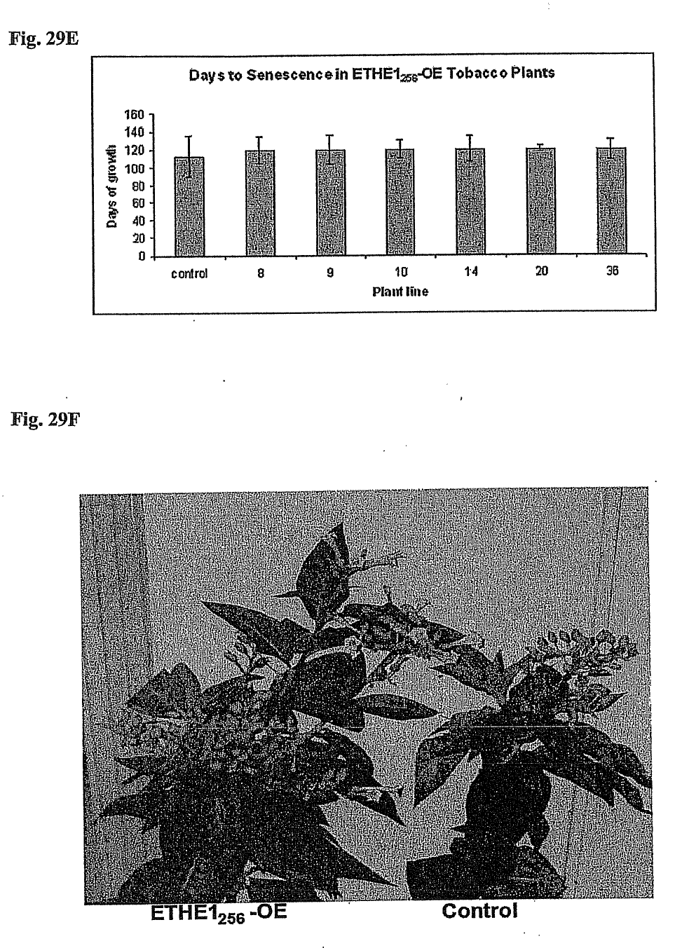

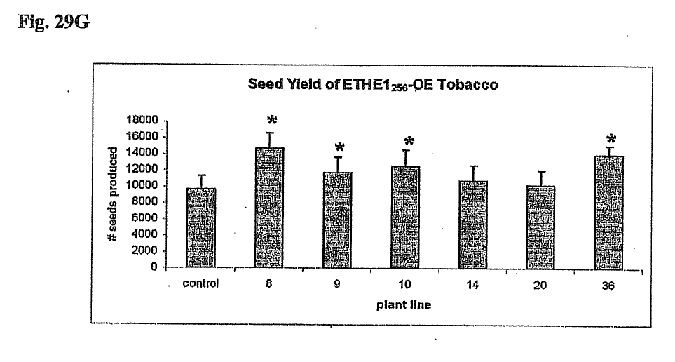

To determine if the growth enhancing properties observed from the over-expression of AtETHE1₂₅₆ in Arabidopsis plants can be replicated in other species, an AtETHE1₂₅₆ over-expression construct was introduced into tobacco plants. Transgenic plants were identified through kanamycin resistance and confirmed through PCR. Six lines were identified as positive for both. RT-PCR was performed on the individual lines and all AtETHE1₂₅₆-OE transgenic plants showed increased levels of the AtETHE1 transcript in comparison to wild type plants, with tobacco lines AtETHE1₂₅₆-OE 8, 9, 10, and 36 showing the greatest increase in mRNA levels (FIG. 29A). The phenotypes described below represent data from two generations of the six lines and include data from 15 plants from each line.

Tobacco plants over-expressing AtETHE1₂₅₆ displayed phenotypes similar to those observed in Arabidopsis ETHE1₂₅₆-OE lines. In general, plants that had higher levels of AtETHE1 mRNA differed most from wild-type tobacco plants. Consistent with the phenotype observed in Arabidopsis ETHE1₂₅₆-OE plants, tobacco transgenic lines that over-express AtETHE1₂₅₆ bolted on average 16+/−3 days after germination on soil compared to 21+/−1 days in tobacco plants containing an empty vector control (FIG. 29 B-C). Likewise, AtETHE1₂₅₆-OE tobacco plants flowered earlier, on average of 61+/−10 days after germination, in contrast to the 77+/−12 days after germination observed in the control plants (FIG. 29D). Interestingly, in contrast to the increased time to senescence observed in AtETHE1₂₅₆-OE Arabidopsis plants, no significant difference was observed in the time to senescence between AtETHE1₂₅₆-OE tobacco plants and the empty vector control (FIG. 29E). Perhaps the most dramatic phenotype of AtETHE1₂₅₆-OE tobacco plants was the increase in overall seed yield (FIG. 29F-G). AtETHE1₂₅₆-OE tobacco plants had on average a 22% increase in seed yield (AtETHE1₂₅₆-OE: 12,335 seeds: Control: 9,682 seeds) (FIG. 29F-G). The most dramatic increase in seed yield was observed in line 8, which exhibited a 34% average overall increase in seed yield.

The increase in seed yield appears to be due to both a shorter time to flowering and a loss of apical dominance in tobacco AtETHE1₂₅₆-OE plants (FIG. 28F). Consistent with our prediction that over-expression of the AtETHE1₂₅₆ protein may be altering cytokinin levels, it is well established that cytokinins along with auxin are capable of controlling apical dominance. High cytokinin to auxin ratios have been shown to result in a loss of apical dominance resulting in the production of more lateral branching. Our studies on transgenic tobacco plants that over-express AtETHE1₂₅₆ demonstrate that the enhanced growth properties observed in Arabidopsis can be conferred to other plants. Without wishing to be bound by theory, our results further suggest that cytoplasmic AtETHE1 may increase cytokinin levels.

### CONCLUDING REMARKS

In the results presented here we demonstrated that Arabidopsis ETHE1 contains a 38 amino acid leader sequence that is essential for its proper processing and native localization in the mitochondrion. Over-expression of native AtETHE1 in Arabidopsis resulted in no obvious phenotypic changes under normal growth conditions. Surprisingly, we found that high levels of cytosolic AtETHE1 leads to changes in the growth properties of both Arabidopsis and tobacco. Specifically, plants that over-express AtETHE1₂₅₆ exhibit resistance to exogenous valine, flower earlier and longer, have a more upright growth habit, and produce more seed. These affects are not observed in plants that over-express AtETHE1₂₉₄ suggesting that cytosolic AtETHE1 may catalyze a reaction not normally catalyzed by the native mitochondrial enzyme or that is has access to a substrate not found in the mitochondrion. The phenotypes exhibited by AtETHE1₂₅₆′ OE plants resemble those of plants containing mutations that increase CK levels, suggesting that cytoplasmic AtETHE1 may indirectly alter CK levels within the plant. The natural AtETHE1 substrate is not yet known. Finally, we have shown that cytoplasmic over-expression of AtETHE1 can enhance the growth properties of both Arabidopsis and tobacco plants and increase seed yield. This raises the possibility that AtETHE1 could ultimately be used to increase seed yield in agriculturally important plants.

The present invention should not be considered limited to the specific examples described above, but rather should be understood to cover all aspects of the invention. Various modifications, equivalent processes, as well as numerous structures and devices to which the present invention may be applicable will be readily apparent to those of skill in the art.

1. A transgenic plant comprising a polynucleotide selected from the group consisting of:
(i) a polynucleotide encoding an ETHE1 polypeptide or a functional fragment thereof;
(ii) a polynucleotide that is fully complementary to the polynucleotide of (i); and
(iii) a polynucleotide that hybridizes under stringent conditions to the polynucleotide sequence of (i) or (ii);
wherein the polynucleotide encodes an ETHE1 polypeptide lacking a mitochondrial leader sequence and having at least 85% identity with the sequence of AtETHE1 of SEQ ID NO: 46 and
wherein the transgenic plant exhibits at least one improved phenotypic characteristic as compared to a control plant not transformed with said polynucleotide.

2. The transgenic plant of claim 1, wherein the improved phenotypic characteristic comprises: bolting rate; speed of growth, including flowering time; longevity; onset of senescence; plant size; plant biomass; vigor; thickness and uprightness of the stem; yield; stress tolerance; pathogen tolerance or a combination thereof.

3. The transgenic plant of claim 1, wherein the polynucleotide encodes an ETHE1 polypeptide having at least 90% identity with the sequence of AtETHE1 of SEQ ID NO: 46.

4. The transgenic plant of claim 1, wherein the polynucleotide encodes an ETHE1 polypeptide having at least 95% identity with the sequence of AtETHE1 of SEQ ID NO: 46.

5. The transgenic plant of claim 1, wherein the polynucleotide encodes an ETHE1 polypeptide having 100% identity with the sequence of AtETHE1 of SEQ ID NO: 46.

6. The transgenic plant of claim 1, wherein the polynucleotide encodes an ETHE1 polypeptide which is at least 220 amino acids in length and comprises one or more of the following features:
(a) comprise the sequence SEQ ID NO: 46, which includes all conservative residues shown in Table 1;
(b) comprise the amino acid residues corresponding to: H72, H74, D76, H77, H128, D153 and H194 in the AtETHE1 sequence of SEQ ID NO: 46;
(c) comprise ETHE1 β-lactamase fold motif HxHxDH x₍₄₉₋₅₀₎ GHT x₍₁₄₋₂₀₎ FTGDx₍₄₀₎A/GHDY (SEQ ID NO: 1);
(d) comprise a tyrosine at the position equivalent to 29 in AtETHE1 (Y29), a threonine at the position equivalent to 129 in AtETHE1 (T129), a cysteine at the position equivalent to 160 in AtETHE1 (C160), an arginine at the position equivalent to 162 in AtETHE1 (R162) and a leucine at the position equivalent to 184 in AtETHE1 (L184); or
(e) comprise one or more of the conserved regions as shown in Table 1

7. The transgenic plant of claim 1, wherein the polynucleotide encodes a functional fragment of the ETHE1 polypeptide which is at least 200 amino acids in length and comprises one or more of:
(a) a deletion and/or substitution of 1 to 16 amino acids corresponding to those located at the N terminus of the AtETHE1 polypeptide sequence of SEQ ID NO: 46;
(b) a deletion and/or substitution of 1 to 9 amino acids corresponding to those located at the C terminus of the AtETHE1 polypeptide sequence of SEQ ID NO: 46;
(c) a deletion and/or substitution of amino acids corresponding to those at positions 36, 37, 139, 140, 141, 144, 145, and/or 146 of the AtETHE1 polypeptide sequence of SEQ ID NO: 46; and/or
(d) addition of one or more amino acids between amino acid residues corresponding to positions 102 and 103, and/or between amino acid residues corresponding to positions 217 and 218 of the AtETHE1 polypeptide sequence of SEQ ID NO: 46.

8. The transgenic plant of claim 1, wherein the polynucleotide encoding an ETHE1 polypeptide is obtained from a plant, an animal, or a bacterium.

9. The transgenic plant of claim 1, wherein the plant is a dicot.

10. The transgenic plant of claim 9, wherein the improved phenotypic characteristic comprises: bolting rate; speed of growth, including flowering time; longevity; onset of senescence; plant size; plant biomass; vigor; thickness and uprightness of the stem; yield; stress tolerance; pathogen tolerance or a combination thereof.

11. The transgenic plant of claim 1, wherein the polynucleotide further comprises a promoter operably linked to the polynucleotide sequence of (i), (ii) or (iii).

12. The transgenic plant of claim 11, wherein the promoter is 35S promoter.

13. A method of increasing the speed of growth, size, or seed yield of a plant by transforming the plant with the expression vector or cassette comprising:
(i) a polynucleotide encoding an ETHE1 polypeptide or a functional fragment thereof; (ii) a polynucleotide that is fully complementary to the polynucleotide of (i); or (iii) a polynucleotide that hybridizes under stringent conditions to the polynucleotide of (i) or (ii); and
wherein the polynucleotide encodes an ETHE1 polypeptide lacking a mitochondrial leader sequence and having at least 85% identity with the sequence of AtETHE1 of SEQ ID NO: 46.

14. The method of claim 13, wherein the plant is a dicot.

15. The method of claim 13, wherein the polynucleotide encodes an ETHE1 polypeptide having at least 90% identity with the sequence of AtETHE1 of SEQ ID NO: 46.

16. The method of claim 13, wherein the polynucleotide encodes an ETHE1 polypeptide having at least 95% identity with the sequence of AtETHE1 of SEQ ID NO: 46.

17. The method of claim 13, wherein the polynucleotide encodes an ETHE1 polypeptide that has the sequence of AtETHE1 of SEQ ID NO: 46.

18. The method claim 13, further comprising selfing the transgenic plant or crossing the transgenic plant with a second plant, thereby producing progeny with an improved phenotypic characteristic.

19. A transgenic plant transformed with a gene encoding a polypeptide that regulates expression of ETHE1 polynucleotide, wherein said transgenic plant exhibits an improved phenotypic characteristic as compared to a control plant not transformed with said gene.

20. A method of selecting or identifying a plant having an improved phenotypic characteristic, the method comprising detecting the level of expression or activity of ETHE1 polynucleotide or polypeptide, wherein detecting an increase in the expression level of ETHE1 polynucleotide or polypeptide, or an increase in the activity level of ETHE1 polypeptide, is indicative of the plant having an improved phenotypic characteristic as compared to a control plant where the expression or activity level of ETHE1 polynucleotide or polypeptide is not increased.


SEQUENCE LISTING (77 sequences)

SEQ ID NO: 1 - protein, 127 aa, Artificial Sequence
  Description of Artificial Sequence: Synthetic construct
  MOD_RES (2)..(2): any amino acid
  MOD_RES (4)..(4): any amino acid
  MOD_RES (7)..(56): region may encompass 49-50 variable amino acids
  MOD_RES (60)..(79): region may encompass 14-20 variable amino acids
  MOD_RES (84)..(123): any amino acid
  MOD_RES (124)..(124): Ala or Gly
HXHXDHXXXXXXXXXXXXXXXXXXXXXXXXXXXXXXXXXXXXXXXXXXXXXXXXXXGHTXXXXXXXXXXXXXXXXXXXXFTGDXXXXXXXXXXXXXXXXXXXXXXXXXXXXXXXXXXXXXXXXXHDY

SEQ ID NO: 2 - protein, 235 aa, Artificial Sequence
  Description of Artificial Sequence: Synthetic construct
  MOD_RES (3)..(3): any amino acid
  MOD_RES (5)..(7): any amino acid
  MOD_RES (9)..(9): any amino acid
  MOD_RES (11)..(11): any amino acid
  MOD_RES (16)..(16): any amino acid
  MOD_RES (18)..(24): region may encompass 5-7 variable amino acids
  MOD_RES (26)..(26): any amino acid
  MOD_RES (32)..(36): any amino acid
  MOD_RES (39)..(40): any amino acid
  MOD_RES (42)..(44): any amino acid
  MOD_RES (48)..(54): any amino acid
  MOD_RES (57)..(57): any amino acid
  MOD_RES (62)..(62): any amino acid
  MOD_RES (64)..(73): region may encompass 9-10 variable amino acids
  MOD_RES (75)..(87): region may encompass 12-13 variable amino acids
  MOD_RES (90)..(94): any amino acid
  MOD_RES (97)..(100): any amino acid
  MOD_RES (102)..(110): any amino acid
  MOD_RES (115)..(134): region may encompass 14-20 variable amino acids
  MOD_RES (139)..(140): any amino acid
  MOD_RES (144)..(144): any amino acid
  MOD_RES (152)..(152): any amino acid
  MOD_RES (154)..(160): any amino acid
  MOD_RES (162)..(166): any amino acid
  MOD_RES (168)..(168): any amino acid
  MOD_RES (171)..(175): any amino acid
  MOD_RES (178)..(178): any amino acid
  MOD_RES (182)..(182): any amino acid
  MOD_RES (184)..(185): any amino acid
  MOD_RES (187)..(191): any amino acid
  MOD_RES (194)..(196): any amino acid
  MOD_RES (198)..(198): any amino acid
  MOD_RES (200)..(208): region may encompass 7-9 variable amino acids
  MOD_RES (210)..(212): any amino acid
  MOD_RES (214)..(214): any amino acid
  MOD_RES (217)..(217): any amino acid
  MOD_RES (219)..(220): any amino acid
  MOD_RES (222)..(223): any amino acid
  MOD_RES (226)..(227): any amino acid
  MOD_RES (232)..(233): any amino acid
RQXFXXXSXTXTYLLXDXXXXXXXAXLIDPVXXXXXRDXXLXXXLGLXXXXXXXTHXHADHXTXXXXXXXXXXGXXXXXXXXXXXXXADXXXXXGDXXXXGXXXXXXXXXPGHTXXXXXXXXXXXXXXXXXXXXFTGDXXLIRXCGRTDFQXGXXXXXXXSXXXXXFXLPXXXXXYPXHDYXGXXVXXXXXEEXXXNXRXXXXXXXXXFXXXMXNLXLXXPXXIDXXVPANXXCG

SEQ ID NO: 3 - protein, 10 aa, Artificial Sequence
  Description of Artificial Sequence: Synthetic peptide
  MOD_RES (2)..(2): Ser or Cys
  MOD_RES (4)..(4): Phe or Tyr
  MOD_RES (9)..(9): Ala or Gly
SXTXTYLLXD

SEQ ID NO: 4 - protein, 7 aa, Artificial Sequence
  Description of Artificial Sequence: Synthetic peptide
  MOD_RES (2)..(2): Leu or Val
AXLIDPV

SEQ ID NO: 5 - protein, 10 aa, Artificial Sequence
  Description of Artificial Sequence: Synthetic peptide
  MOD_RES (3)..(4): Leu or Ala
  MOD_RES (6)..(7): Ile or Leu
RDXXLXXLGL

SEQ ID NO: 6 - protein, 16 aa, Artificial Sequence
  Description of Artificial Sequence: Synthetic peptide
  MOD_RES (1)..(1): Asn or Asp
  MOD_RES (4)..(4): Val or Cys
  MOD_RES (9)..(9): Val or Ile
  MOD_RES (11)..(11): Gly or Ala
  MOD_RES (12)..(12): Thr or Ser
  MOD_RES (13)..(13): Gly or Trp
  MOD_RES (14)..(14): Leu, Val, Ile or Met
  MOD_RES (15)..(15): Leu or Ile
  MOD_RES (16)..(16): Lys, Arg or Asn
XTHXHADHXTXXXXXX

SEQ ID NO: 7 - protein, 13 aa, Artificial Sequence
  Description of Artificial Sequence: Synthetic peptide
  MOD_RES (1)..(1): Thr or Ser
  MOD_RES (6)..(6): any amino acid
  MOD_RES (7)..(7): Gly or Asp
  MOD_RES (8)..(8): Cys or Ser
  MOD_RES (9)..(9): Val, Leu or Ile
  MOD_RES (10)..(10): Thr or Ser
  MOD_RES (11)..(11): Tyr, Phe or Leu
  MOD_RES (12)..(12): Val or Leu
  MOD_RES (13)..(13): Thr or Leu
XPGHTXXXXXXXX

SEQ ID NO: 8 - protein, 5 aa, Artificial Sequence
  Description of Artificial Sequence: Synthetic peptide
  MOD_RES (1)..(1): Thr or Ser
XPGHT

SEQ ID NO: 9 - protein, 20 aa, Artificial Sequence
  Description of Artificial Sequence: Synthetic peptide
  MOD_RES (1)..(1): Ala or Val
  MOD_RES (6)..(6): Ala or Cys
  MOD_RES (7)..(7): Val or Leu
  MOD_RES (11)..(11): Gly or Ala
  MOD_RES (19)..(19): any amino acid
XFTGDXXLIRXCGRTDFQXG

SEQ ID NO: 10 - protein, 13 aa, Artificial Sequence
  Description of Artificial Sequence: Synthetic peptide
  MOD_RES (1)..(1): Ala or Cys
  MOD_RES (2)..(2): Val or Leu
  MOD_RES (6)..(6): Gly or Ala
XXLIRXCGRTDFQ

SEQ ID NO: 11 - protein, 18 aa, Artificial Sequence
  Description of Artificial Sequence: Synthetic peptide
  MOD_RES (2)..(2): any amino acid
  MOD_RES (3)..(3): Glu or Thr
  MOD_RES (5)..(5): Ile, Ser or Thr
  MOD_RES (6)..(6): Thr or Ser
  MOD_RES (7)..(7): Val or Ile
  MOD_RES (8)..(8): any amino acid
  MOD_RES (11)..(13): any amino acid
  MOD_RES (17)..(17): Leu or Val
GXXVXXXXEEXXXNPRXT

SEQ ID NO: 12 - protein, 20 aa, Artificial Sequence
  Description of Artificial Sequence: Synthetic peptide
  MOD_RES (1)..(1): Thr or Cys
  MOD_RES (2)..(2): Leu or Phe
  MOD_RES (3)..(3): Ile, Leu or Val
  MOD_RES (6)..(6): Ala or Gly
  MOD_RES (10)..(10): any amino acid
  MOD_RES (12)..(12): any amino acid
  MOD_RES (13)..(13): Glu or Thr
  MOD_RES (15)..(15): Ile, Ser or Thr
  MOD_RES (16)..(16): Thr or Ser
  MOD_RES (17)..(17): Val or Ile
  MOD_RES (18)..(18): any amino acid
XXXYPXHDYXGXXVXXXXEE

SEQ ID NO: 13 - protein, 25 aa, Artificial Sequence
  Description of Artificial Sequence: Synthetic peptide
  MOD_RES (1)..(1): Ile, Val, Tyr or Leu
  MOD_RES (3)..(3): any amino acid
  MOD_RES (6)..(6): any amino acid
  MOD_RES (8)..(9): any amino acid
  MOD_RES (11)..(12): any amino acid
  MOD_RES (15)..(15): any amino acid
  MOD_RES (16)..(16): Ala or Ser
  MOD_RES (21)..(22): any amino acid
  MOD_RES (25)..(25): Leu, Val or Ile
XMXNLXLXXPXXIDXXVPANXXCGX

SEQ ID NO: 14 - protein, 5 aa, Arabidopsis thaliana
MGSSS

SEQ ID NO: 15 - protein, 5 aa, Arabidopsis thaliana
PSQAN

SEQ ID NO: 16 - DNA, 31 bp, Artificial Sequence
  Description of Artificial Sequence: Synthetic primer
tcttctcatatgaagcttctctttcgtcaac

SEQ ID NO: 17 - DNA, 38 bp, Artificial Sequence
  Description of Artificial Sequence: Synthetic primer
gagtcgactcgagctctagatctttttttttttttttt

SEQ ID NO: 18 - protein, 7 aa, Artificial Sequence
  Description of Artificial Sequence: Synthetic peptide
  MOD_RES (4)..(4): Leu or Phe
  MOD_RES (5)..(5): Phe or Tyr
CGKXXEG

SEQ ID NO: 19 - protein, 9 aa, Artificial Sequence
  Description of Artificial Sequence: Synthetic peptide
CGRTDFQEG

SEQ ID NO: 20 - protein, 7 aa, Artificial Sequence
  Description of Artificial Sequence: Synthetic peptide
MKLLFRQ

SEQ ID NO: 21 - DNA, 22 bp, Artificial Sequence
  Description of Artificial Sequence: Synthetic primer
tggacaagactgtggatagaga

SEQ ID NO: 22 - DNA, 22 bp, Artificial Sequence
  Description of Artificial Sequence: Synthetic primer
agtaagttgtgttgtgtcacac

SEQ ID NO: 23 - protein, 6 aa, Artificial Sequence
  Description of Artificial Sequence: Synthetic peptide
  MOD_RES (3)..(3): any amino acid
  MOD_RES (5)..(5): any amino acid
THXHXD

SEQ ID NO: 24 - protein, 17 aa, Artificial Sequence
  Description of Artificial Sequence: Synthetic peptide
MGSSSSFSSSSSKLLFR

SEQ ID NO: 25 - protein, 21 aa, Artificial Sequence
  Description of Artificial Sequence: Synthetic peptide
MGSSSSFSSSSSKLLFRQLFE

SEQ ID NO: 26 - protein, 10 aa, Artificial Sequence
  Description of Artificial Sequence: Synthetic peptide
SSTFTYLLAD

SEQ ID NO: 27 - protein, 17 aa, Artificial Sequence
  Description of Artificial Sequence: Synthetic peptide
KPALLIDPVDKTDVDRD

SEQ ID NO: 28 - protein, 25 aa, Artificial Sequence
  Description of Artificial Sequence: Synthetic peptide
ASGSKALDFLEPGDKRSIGDIVLER

SEQ ID NO: 29 - protein, 10 aa, Artificial Sequence
  Description of Artificial Sequence: Synthetic peptide
ADLFLEPGDK

SEQ ID NO: 30 - protein, 21 aa, Artificial Sequence
  Description of Artificial Sequence: Synthetic peptide
ADLFLEPGDKRSIGDIVLEVR

SEQ ID NO: 31 - protein, 10 aa, Artificial Sequence
  Description of Artificial Sequence: Synthetic peptide
KVSIGDIYLE

SEQ ID NO: 32 - protein, 11 aa, Artificial Sequence
  Description of Artificial Sequence: Synthetic peptide
VSIGDIVLEVR

SEQ ID NO: 33 - protein, 11 aa, Artificial Sequence
  Description of Artificial Sequence: Synthetic peptide
MAFTGDAVLIR

SEQ ID NO: 34 - protein, 11 aa, Artificial Sequence
  Description of Artificial Sequence: Synthetic peptide
MAFTGDAVLIR

SEQ ID NO: 35 - protein, 15 aa, Artificial Sequence
  Description of Artificial Sequence: Synthetic peptide
MAFTGDAVLIRGCGR

SEQ ID NO: 36 - protein, 23 aa, Artificial Sequence
  Description of Artificial Sequence: Synthetic peptide
TDFQEGSSDQLESVHSQIFTLPK

SEQ ID NO: 37 - protein, 11 aa, Artificial Sequence
  Description of Artificial Sequence: Synthetic peptide
DTLIVPAHDYK

SEQ ID NO: 38 - protein, 9 aa, Artificial Sequence
  Description of Artificial Sequence: Synthetic peptide
LTKDKETFK

SEQ ID NO: 39 - protein, 9 aa, Artificial Sequence
  Description of Artificial Sequence: Synthetic peptide
LTKDKETFK

SEQ ID NO: 40 - protein, 16 aa, Artificial Sequence
  Description of Artificial Sequence: Synthetic peptide
ETFKTIMSNLNLSYPK

SEQ ID NO: 41 - protein, 12 aa, Artificial Sequence
  Description of Artificial Sequence: Synthetic peptide
TIMSNLNLSYPK

SEQ ID NO: 42 - protein, 12 aa, Artificial Sequence
  Description of Artificial Sequence: Synthetic peptide
TIMSNLNLSYPK

SEQ ID NO: 43 - protein, 11 aa, Artificial Sequence
  Description of Artificial Sequence: Synthetic peptide
VPSQANMDYKD

SEQ ID NO: 44 - DNA, 3427 bp, Arabidopsis thaliana
aggtggatttggataaaagtgcattcgggtcatatgatacattctcctattaaataaatatggtgcactcttatgttattagctaaaatgtttagattctaattcagagtcataccattaataaaattaatattaacaaaataaatagctttttggcaagacggatttggagggacgggtttttgaatcaacattaataaaaaagtaaaatataattaatcgaccgtttcaatacgggttaaatctttaatttattattttttaagaccactaatattaaacatatcaaatcatcctaatttagaaaagattatataaaaccaaaaatgttatgttatgtggtatgtataatgttactatatataaaattaaactatagatataaatgtattagagaatgatacaatttgcaaaacttttctatataaaaaattattcttaaatgttaaaaattactatttaaaaaaaaaaatcatgggacggtaaaaaaattacagaatgagttctattttggaattgggttatatggtggatgtatttgaatcaatatttataaaatttaaaaatattattaatatactgttttaataggggttaaaacttcagttttttaacaattatctcatggattcgtggtataccgttacttaataacaattataaactgtaaaatataaatatttaataaaaataaaatttgcaagttttaatatatattacttttaaaaaataaatcgtcccgcgatataccgcggttaaaatctagtttgatatttatgaagttgtactcaaactcaaaggtcaaaaccaaatgctataatttcatctttgtaatggacctacaaatagaatcataaccaacatcctgactgttgacctaaaccgtggagtccaccagtcctgactgttgacctaaaccgtggagtccaccagacatatgacttgttgtcggcaacactttggtttttagttaagattaagcccaatagccccataagcccattagtgtaggccctattaagtccatctacactcgaaaacttcatcatcatcttcaaggtttaagattggcttcatgaatctccaatcattctcagatgattgtttaatcttatctgataaaaatcatttgcttccataattgttactgaatcgtgcagaactgatcaatcacgggtcccaccacatttgtttcaagaagctattaacgtctccgacaacttcttcttctccaacccaaattcctctcttctcaacctcgtcctctccgttctcctcctccaacatttctccgatcagtgatgggttcgtcttcttctttctcctcttcttcctcgaagcttctctttcgtcaactctttgagaacgaatcttccacctttacttatcttctcgccgacgtttctcatcctgataaacctgctttggtatgtttttgcttcctaaagattgaatttttcgacatgggttttggttttttcgtgtctggtctttgtttttcgaatcctagttctgatcttcatgatgaataataggaactggattcatttagttttgaaaagatcatcaattttggatcaagttatgttctttgttcatctgatttgttgaatttggattgttttatgtgatgtgatgagcagttgattgatccggtggacaagactgtggatagagacttgaaactgattgatgagttaggactaaagcttatctatgctatgaacactcatgttcatgctgatcatgtcactggtactggacttcttaaggtctgttttctcttgcttgatgttatttgtttatatttccatggttttggtttaaatcgaagattcagtgatctatctttgtttgctccacacagacgaagctcccgggtgtgaaatccgttatttcgaaagcaagtggttccaaagctgatttgtttcttgaacctggtgacaaagtatctattggtgatatataccttgaggtatgaaaccacaatttcaaaccggtacaataaatgagaggctagaaagtgaagagtgtacatttgtctcaggttcgtgctacacctggacacactgcaggatgtgttacatatgtgactggtgaaggagctgatcagccccaaccaagaatggcttttaccggggatgctgtactcatccgtggttgtgggaggactgactttcaggtacacaaaacagagaaaaagtcggttattaagaaccataaaccacaatgtagatgcataacatctagattttgtgttttagagtctgcaatgtagatgcataagatctgattttgtatgcataagctctatagattctgtaatgaggcatcgtttaactgtactacgatgttttcctcggtgtttcaggaaggaagctcagatcaactctacgagtctgtacattcacaggcaagtgttttacattccataggcaagtggttcatattttcaaggactctgtttaaacgcttttgttttttcttttaccagatatttacattgccaaaggacacattgatctatcctgctcacgactacaaaggtttcgaggtaatgcaaaacgcaaattcatttacattggcttgtttaatggaaaatatgactcatccacgcaatggtttttacatcgtttctgttaaaattcaggtaagtacagttggagaagagatgcaacacaacccgcgtctaactaaagataaagaaacattcaaaaccattatgtcaagtaagtttcttctacttagtatgatgctaaagtattcatcatctcataaatgaccgccgtttttgctcttgacagatctgaatctgtcgtatccgaagatgattgatgttgcagtaccagcaaatatggtctgtgggttacaagatgtgccttctcaagccaactaaaaaaaactcttacatataatgtttgtctttttatcgatgtcattctatatttaccaaagccaataaaaactcttgagagtaatttctacaataagatttgcttccgttgtagataagttcattagaaaattgtgtaaattagattgaacatgtggaatttgttatgaagatgaagacagtatttgcgtgttcgacttttgtttggtttgttgattgatgtgtcaactacttttcaattaacataaatctacatgagctttttcaaccaccattcgtttatgttctgctgctacgtgtcgaaattgctaaaaccttttaatcttagtgatcaaacaatgactcagcgtgagagagacaatcttcctatctttttcttagtgggaaacttgaaaattcgaccatctcctcaactaatgtcaaatatctaaaggaaaacaaacatgagtaactttactttctgaaaagtatattcacactgtaggttgaaaacaaatggttgaatattatctttgtgtgacaacacaacttact

SEQ ID NO: 45 - DNA, 928 bp, Arabidopsis thaliana
  CDS (34)..(801)
cgttctcctcctccaacatttctccgatcagtgatgggttcgtcttcttctttctcctcttcttcctcgaagcttctctttcgtcaactctttgagaacgaatcttccacctttacttatcttctcgccgacgtttctcatcctgataaacctgctttgttgattgatccggtggacaagactgtggatagagacttgaaactgattgatgagttaggactaaagcttatctatgctatgaacactcatgttcatgctgatcatgtcactggtactggacttcttaagacgaagctcccgggtgtgaaatccgttatttcgaaagcaagtggttccaaagctgatttgtttcttgaacctggtgacaaagtatctattggtgatatataccttgaggttcgtgctacacctggacacactgcaggatgtgttacatatgtgactggtgaaggagctgatcagccccaaccaagaatggcttttaccggggatgctgtactcatccgtggttgtgggaggactgactttcaggaaggaagctcagatcaactctacgagtctgtacattcacagatatttacattgccaaaggacacattgatctatcctgctcacgactacaaaggtttcgaggtaagtacagttggagaagagatgcaacacaacccgcgtctaactaaagataaagaaacattcaaaaccattatgtcaaatctgaatctgtcgtatccgaagatgattgatgttgcagtaccagcaaatatggtctgtgggttacaagatgtgccttctcaagccaactaaaaaaaactcttacatataatgtttgtctttttatcgatgtcattctatatttaccaaagccaataaaaactcttgagagtaatttctacaataagatttgcttccgttgtagataagttcatta

SEQ ID NO: 46 - protein, 256 aa, Arabidopsis thaliana
MGSSSSFSSSSSKLLFRQLFENESSTFTYLLADVSHPDKPALLIDPVDKTVDRDLKLIDELGLKLIYAMNTHVHADHVTGTGLLKTKLPGVKSVISKASGSKADLFLEPGDKVSIGDIYLEVRATPGHTAGCVTYVTGEGADQPQPRMAFTGDAVLIRGCGRTDFQEGSSDQLYESVHSQIFTLPKDTLIYPAHDYKGFEVSTVGEEMQHNPRLTKDKETFKTIMSNLNLSYPKMIDVAVPANMVCGLQDVPSQAN

SEQ ID NO: 47 - protein, 256 aa, Arabidopsis thaliana
MGSSSSFSSSSSKLLFRQLFENESSTFTYLLADVSHPDKPALLIDPVDKTVDRDLKLIDELGLKLIYAMNTHVHADHVTGTGLLKTKLPGVKSVISKASGSKADLFLEPGDKVSIGDIYLEVRATPGHTAGCVTYVTGEGADQPQPRMAFTGDAVLIRGCGRTDFQEGSSDQLYESVHSQIFTLPKDTLIYPAHDYKGFEVITVGEEMQHNPRLTKDKETFKTIMSNLNLSYPKMIDVAVPANMVCGLQDVPSQAN

SEQ ID NO: 48 - protein, 254 aa, Homo sapiens
MAEAVLRVARRQLSQRGGSGAPILLRQMFEPVSCTFTYLLGDRESREAVLIDPVLETAPRDAQLIKELGLRLLYAVNTHCHADHITGSGLLRSLLPGCQSVISRLSGAQADLHIEDGDSIRFGRFALETRASPGHTPGCVTFVLNDHSMAFTGDALLIRGCGRTDFQQGCAKTLYHSVHEKIFTLPGDCLIYPAHDYHGFTVSTVEEERILNPRLTLSCEEFVKIMGNLNLPKPQQIDFAVPANMRCGVQTPTA

SEQ ID NO: 49 - protein, 258 aa, Arabidopsis thaliana
MKIFHVPCLQDNYSYLIIDESTGDAAVVDPVDPEKVIASAEKHQAKIKFVLTTHHHWDHAGGNEKIKQLVPDIKVYGGSLDKVKGCTDAVDNGDKLTLGQDINILALHTPCHTKGHISYYVNGKEGENPAVFTGDTLFVAGCGKFFEGTAEQMYQSLCVTLAALPKPTQVYCGHEYTVKNLEFALTVEPNNGKIQQKLAWARQQRQADLPTIPSTLEEELETNPFMRVDKPEIQEKLGCKSPIDTMREVRNKKDQWRG

SEQ ID NO: 50 - protein, 313 aa, Arabidopsis thaliana
MKQIGGQLCVWPGLRQLCLRKSLLYGVMWLLSMPLKTLRGARKTLKITHFCSISNMPSSLKIELVPCSKDNYAYLLHDEDTGTVGVVDPSEAAPVIEALSRKNWNLTYILNTHHHDDHIGGNAELKERYGAKVIGSAVDKDRIPGIDILLKDSDKWMFAGHEVRILDTPGHTQGHISFYFPGSATIFTGDLIYSLSCGTLSEGTPEQMLSSLQKIVSLPDDTNIYCGRENTAGNLKFALSVEPKNETLQSYATRVAHLRSQGLPSIPTTVKVEKACNPFLRISSKDIRKSLSIPDSATEAEALRRIQRARDRF

SEQ ID NO: 51 - protein, 323 aa, Arabidopsis thaliana
MQTISKASSATSFFRCSRLSSQPCVRQLNIRKSLVCRVMKLVSSPLRTLRGAGKSIRVSKFCSVSNVSSLQIELVPCLKDNYAYILHDEDTGTVGVVDPSEAEPIIDSLKRSGRNLTYILNTHHHYDHTGGNLELKDRYGAKVIGSAMDKDRIPGIDMALKDGDKWMFAGHEVHVMDTPGHTKGHISLYFPGSRAIFTGDTMFSLSCGKLFEGTPKQMLASLQKITSLPDDTSIYCGHEYTLSNSKFALSLEPNNEVLQSYAAHVAELRSKKLPTIPTTVKMEKACNPFLRSSNTDIRRALRIPEAADEAEALGIIRKAKDDF

SEQ ID NO: 52 - protein, 257 aa, Arabidopsis thaliana
MGSSSSFSSSSSKLLFRQLFENESSTFTYLLADVSHPDKPALLIDPVDKTVDRDLKLIDELGLKLIYAMNTHVHADHVTGTGLLKTKLPGVKSVISKASGSKADLFLEPGDKVSIGDIYLEVRATPGHTAGCVTYVTGEGADQPQPRMAFTGDAVLIRGCGRTDFQEGGSSDQLYESVHSQIFTLPKDTLIYPAHDYKGFEVITVGEEMQHNPRLTKDKETFKTIMSNLNLSYPKMIDVAVPANMVCGLQDVPSQAN

SEQ ID NO: 53 - protein, 255 aa, Homo sapiens
MAEAVLRVARRQLSQRGGSGAPILLRQMFEPVSCTFTYLLGDRESREAVLIDPVLETAPRDAQLIKELGLRLLYAVNTHCHADHITGSGLLRSLLPGCQSVISRLSGAQADLHIEDGDSIRFGRFALETRASPGHTPGCVTFVLNDHSMAFTGDALLIRGCGRTDFQQGGCAKTLYHSVHEKIFTLPGDCLIYPAHDYHGFTVSTVEEERTLNPRLTLSCEEFVKIMGNLNLPKPQQIDFAVPANMRCGVQTPTA

SEQ ID NO: 54 - protein, 255 aa, Arabidopsis thaliana
MQIELVPCLKDNYAYILHDEDTGTVGVVDPSEAEPIIDSLKRSGRNLTYILNTHHHYDHTGGNLELKDRYGAKVIGSAMDKDRIPGIDMALKDGDKWMFAGHEVHVMDTPGHTKGHISLYFPGSRAIFTGDTMFSLSCGKLFEGGTPKQMLASLQKITSLPDDTSIYCGHEYTLSNSKFALSLEPNNEVLQSYAAHVAELRSKKLPTIPTTVKMEKACNPFLRSSNTDIRRALRIPEAADEAEALGIIRKAKDDF

SEQ ID NO: 55 - protein, 261 aa, Homo sapiens
MKVEVLPALTDNYMYLVIDDETKEAAIVDPVQPQKVVDAARKHGVKLTTVLTTHHHWDHAGGNEKLVKLESGLKVYGGDDRIGALTHKITHLSTLQVGSLNVKCLATPCHTSGHICYFVSKPGGSEPPAVFTGDTLFVAGCGKFYEGGTADEMCKALLEVLGRLPPDTRVYCGHEYTINNLKFARHVEPGNAAIREKLAWAKEKYSIGEPTVPSTLAEEFTYNPFMRVREKTVQQHAGETDPVTTMRAVRREKDQFKMPRD

SEQ ID NO: 56 - protein, 294 aa, Arabidopsis thaliana
MVMTHFSRLRQLLLLQPKFLSSQPRPLRSPPPTFLRSVMGSSSSFSSSSSKLLFRQLFENESSTFTYLLADVSHPDKPALLIDPVDKTVDRDLKLIDELGLKLIYAMNTHVHADHVTGTGLLKTKLPGVKSVISKASGSKADLFLEPGDKVSIGDIYLEVRATPGHTAGCVTYVTGEGADQPQPRMAFTGDAVLIRGCGRTDFQEGSSDQLYESVHSQIFTLPKDTLIYPAHDYKGFEVITVGEEMQHNPRLTKDKETFKTIMSNLNLSYPKMIDVAVPANMVCGLQDVPSQAN

SEQ ID NO: 57 - protein, 254 aa, Homo sapiens
MAEAVLRVARRQLSQRGGSGAPILLRQMFEPVSCTFTYLLGDRESREAVLIDPVLETAPRDAQLIKELGLRLLYAVNTHCHADHITGSGLLRSLLPGCQSVISRLSGAQADLHIEDGDSIRFGRFALETRASPGHTPGCVTFVLNDHSMAFTGDALLIRGCGRTDFQQGCAKTLYHSVHEKIFTLPGDCLIYPAHDYHGFTVSTVEEERTLNPRLTLSCEEFVKIMGNLNLPKPQQIDFAVPANMRCGVQTPTA

SEQ ID NO: 58 - protein, 254 aa, Mus musculus
MASAVVRVAGRRLSQQSASGAPVLLRQMFEPKSCTYTYLLGDRESREAVLIDPVLETAHRDAQLIKELGLKLLYAVNTHCHADHITGTGVLRSLLPGCQSVISRLSGAQADLHIGEGDSIRFGRFALETRASPGHTPGCVTFVLNDQSMAFTGDALLIRGCGRTDFQQGCAKTLYHSVHEKIFTLPGNCLIYPAHDYHGLTVSTVEEERTLNPRLTLSCEEFIKVMDNLNLPKPQQIDIAVPANMRCGVQTPPS

SEQ ID NO: 59 - protein, 255 aa, Xenopus laevis
MLFVALKQAALGQCRRYSAMAASSGLVFRQLFEPVSCTYTYLLADKNTKEAILIDPVLDKAERDAKLIKDLGLNMIYAANTHCHADHITGTGILKKLLPGCKSVISKDSGARADLYIQEGDQIKFGKFWVEARSTPGHTDGCLTYVLNDKSMAFTGDALLIRGCGRTDFQQGCPKTLYHSVHSKIFSLPGNCLLYPGHDYTGQTVSSVEEEKRLNPRLTKDEAEFVKIMNNLNLPKPKQIDVAVPANLKCGIQDP

SEQ ID NO: 60 - protein, 255 aa, Xenopus tropicalis
MLFVALRQAALSQCRRYSAMAASNGLVFRQLFEPVSCTYTYLLADKNTKEAILIDPVLEKAERDAKLIKDLGFNMIFAANTHCHADHITGTGVLKKLLPGCKSVISKDSGARADVYIQEGDQIKFGKFWVEARSTPGHTDGCLTYVLNDQSMAFTGDALLIRGCGRTDFQQGCPKTLYHSVHTKIFSLPDSCLLYPGHDYTGQTVSSVEEEKRLNPRLTKDEAEFVKIMNNLNLPKPKQIDVAVPANLKCGIQDP

SEQ ID NO: 61 - protein, 279 aa, Danio rerio
MSSALLNRLKPAVSSALRLWSSRPTIPPGLSLRYAASVRLYSGLMEPAAPLFRQLFESESCTYTYLLADPDTREAVLIDPVLETVDRDLQLIQQLGLNLTVALNTHCHADHITGTGLLKKKVFGLKSGISKHSGAAADIQLSDGDSITFGKHCLMVRETPGHTDGCVTYVTGDQRMAFTGDALLIRGCGRTDFQQGSPHRLYESVHQKIFSLPGHCFIYPAHDYKGQTVSTVDEEKKFNPRLTKTVEEFVKIMDNLNLPKPKKIDISVPANLVCGLHDI

SEQ ID NO: 62 - protein, 305 aa, Oryza sativa
MVALLRSCRRLIPHLSACAAAAPSSSSSCAPRARPISRGLRLLPVVLAMAGYSSGSAAEGRRLLFRQLFEKESSTYTYLLADVGDPEKPAVLIDPVDRTVDRDLNLIKELGLKLVYAMNTHVHADHVTGTGLIKTKLPGVKSVIAKVSKAKADHFIEHGDKIYFGNLFLEVRSTPGHTAGCVTYVTGEGDDQPSPRMAFTGDALLIRACGRTDFQGGSSDELYESVHSQIFTLPKDTLLYPGHDYKGFTVSTVEEEVAYNARLTKDKETFKKIMDNLNLAYPKMIDVAVPANLLCGIQDPPPSKV

SEQ ID NO: 63 - protein, 357 aa, Burkholderia phytofirmans
MLLFRQLFDQQSSTYTYLLADSTTREAVLIDPVFEQVRRDAALIEELGLHLLYTIDTHVHADHVTGAWMLNRRIGSRIAISAASGAEGADRYLSHGDKVEFGTRYLTVRATPGHTDGCITLVLDNETMAFTGDCLLIRGTGRTDFQRGDAHTMFRAVHGQIFTLPTACLLYPAHDYRGLTVTSVGEERRFNPRLGGELCEEDFTGYMTNLHLPHPKQIDVAVPANLKCGLAASVPTQMTEPDWAPLTCSFAGIWEINAQWLEENLRAVEIVDVREPEEFNGPLGRIPAARLISLGELAGRTAELTKDRPIVTVCRAGGRSAQATVMLRQAGFERVANLPGGMLRWRAEGRVVENGSV

SEQ ID NO: 64 - protein, 237 aa, Myxococcus xanthus
MLFRQLFDTTSSTYTYLLGDEEQGTALLIDPVAEKLDRDLTLLRELGLSLTHALDTHVHADHVTASGLLRARTGAKVVSGITGAPCADIHVKHGDTLRAGTFTLQVLATPGHTDDSVSYLLGDRVFTGDALLIRGNGRTDFQNGNAGTLYDSITQVLFALPDETLVYPAHDYRGLTVTTIGEEKRHNPRVAGRSRDGFIQLMNNLGLPKPKLIDVAVPANRACGLTAPVRARGSFTH

SEQ ID NO: 65 - protein, 294 aa, Arabidopsis thaliana; identical to SEQ ID NO: 56

SEQ ID NO: 66 - protein, 254 aa, Homo sapiens; identical to SEQ ID NO: 57

SEQ ID NO: 67 - protein, 258 aa, Arabidopsis thaliana; identical to SEQ ID NO: 49

SEQ ID NO: 68 - protein, 323 aa, Arabidopsis thaliana; identical to SEQ ID NO: 51

SEQ ID NO: 69 - protein, 220 aa, Stenotrophomonas maltophilia
VLLDGGMPQMAGHLISNMKVRGVAPQDLRLILLSHAHADHAGPVAELKRRTGAKVAANAESAVLLARGGSDDLHFGDGITYPPASADRIVMDGEMITVGGIAFTAHFMPGHTPGSTAWTWTDTREGKPVRIAYADSLSAPGYQLQGNPRYPHLIEDYRHSLATVRALPCDVLLTPHPGASNWDYAAGSKASAKALTCKAYADAAEQKFDAQLAKETAGAR

SEQ ID NO: 70 - protein, 254 aa, Homo sapiens; identical to SEQ ID NO: 57

SEQ ID NO: 71 - protein, 294 aa, Arabidopsis thaliana; identical to SEQ ID NO: 56

SEQ ID NO: 72 - protein, 258 aa, Arabidopsis thaliana; identical to SEQ ID NO: 49

SEQ ID NO: 73 - protein, 324 aa, Arabidopsis thaliana
MQTISKASSATSFFRCSRKLSSQPCVRQLNIRKSLVCRVMKLVSSPLRTLRGAGKSIRVSKFCSVSNVSSLQIELVPCLKDNYAYILHDEDTGTVGVVDPSEAEPIIDSLKRSGRNLTYILNTHHHYDHTGGNLELKDRYGAKVIGSAMDKDRIPGIDMALKDGDKWMFAGHEVHVMDTPGHTKGHISLYFPGSRAIFTGDTMFSLSCGKLFEGTPKQMLASLQKITSLPDDTSIYCGHEYTLSNSKFALSLEPNNEVLQSYAAHVAELRSKKLPTIPTTVKMRVDKPEILRSSNTDIRRALRIPEAADEAEALGIIRKAKDDF

SEQ ID NO: 74 - protein, 290 aa, Stenotrophomonas maltophilia
MRSTLLAFALAVALPIARASGAETPLPQLRAYTVDASWLQPHAPLQIADHTWQIGTQDLTALLVQTADGAVLLDGGMPQMAGHLISNMKVRGVAPQDLRLILLSHAHADHAGPVAELKRRTGAKVAANAESAVLLARGGSDDLHFGDGITYPPASADRIVMDGEMITVGGIAFTAHFMPGHTPGSTAWTWTDTREGKPVRIAYADSLSAPGYQLQGNPRYPHLIEDYRHSLATVRALPCDVLLTPHPGASNWDYAAGSKASAKALTCKAYADAAEQKFDAQLAKETAGAR

SEQ ID NO: 75 - protein, 294 aa, Arabidopsis thaliana; identical to SEQ ID NO: 56

SEQ ID NO: 76 - protein, 258 aa, Homo sapiens; identical to SEQ ID NO: 49

SEQ ID NO: 77 - protein, 317 aa, Arabidopsis thaliana
MVMTHFSRLRQLLLLQLPKFLSSQPRPLRSPPPTFLRSVMGSSSSFSSSSSKLLFRQLFENESSTFTYLLADVSHPDKPALLIDPVDKTVDRDLKLIDELGLKLIYAMNTHVHADHVTGTGLLKTKLPGVKSVISKASGSKADLFLEPGDKVSIGDIYLEVRATPGHTAGCVTYVTGEGADQPQPRMAFTGDAVLIRGCGRTDFQEGSSDQLYESVHSQIFTLPKDTLIYPAHDYKGFEVSTVGEEMQHNPRLTKDKETFKTIMSNLNLSYPKMIDVAVPANMVCGLQDVPSQANMDYKDDDDKLINWSHPQFEKLG